(19) United States
(12) Patent Application Publication
(10) Pub. No.: US 2026/0102415 A1
(43) Pub. Date: Apr. 16, 2026

(54) COMPOSITIONS COMPRISING PRODRUGS OF HYDROXAMATE-BASED COMPOUNDS AND METHODS OF MAKING AND USING SAME

(71) Applicant: Duke University, Durham, NC (US)

(72) Inventors: Pei Zhou, Durham, NC (US); David Gooden, Durham, NC (US)

(21) Appl. No.: 19/116,194

(22) PCT Filed: Oct. 5, 2023
(86) PCT No.: PCT/US2023/076116

§ 371 Date: Mar. 27, 2025

Related U.S. Application Data
(60) Provisional application No. 63/413,307, filed on Oct. 5, 2022.

(51) Int. Cl. A61K 31/6615; A61K 45/06; A61P 31/04; C07F 9/09

(52) U.S. Cl. CPC A61K 31/6615; A61K 45/06; A61P 31/04; C07F 9/097; C07F 9/098

(57) ABSTRACT

The present disclosure describes, in part compositions comprising prodrugs of hydroxamate-based compounds, such as LpxC inhibitors, and methods of making and using same.

### CROSS-REFERENCE TO RELATED APPLICATIONS

This application claims priority to U.S. Provisional Application No. 63/413,307, filed Oct. 5, 2022, the entire content of which is incorporated by reference herein.

### STATEMENT REGARDING FEDERALLY SPONSORED RESEARCH AND DEVELOPMENT

This invention was made with Government support under Federal Grant No. AI139216 awarded by the National Institute of Allergy and Infectious Disease (NIH/NIAID). The Federal Government has certain rights to this invention.

### BACKGROUND

Growing evidence shows that hydroxamate-based compounds exhibit broad-spectrum pharmacological properties including antimicrobial and anti-tumor activity. Many of these compounds have been shown to be very promising as new therapeutic compounds for treating diseases such as Gram-negative bacterial infections, cancer, and the like.

With regards to antimicrobial activity, some hydroxamate-based compounds have been shown to be potent antibiotics. Gram-negative bacteria are characterized by the presence of a unique outer membrane in their cell envelope. Specifically, the outer membrane consists of phospholipid in the inner leaflet and lipid A in the outer leaflet, which serves as a permeability barrier to shield Gram-negative bacteria from the damage of external detergents and antibiotics. These hydroxamate-based compounds target LpxC, the second enzyme in the pathway, and display impressive antimicrobial activity against susceptible and multidrug-resistant Gram-negative bacteria in vitro and in animal models, highlighting the therapeutic potential of disrupting lipid A biosynthesis as an effective counter measurement to combat drug-resistant Gram-negative infections.

However, hydroxamate-based compounds suffer from formulation issues, such as poor solubility, efficacy, safety and the like. There is a need for further research in addressing the formulation issues associated with these compounds.

### SUMMARY

The Summary is provided to introduce a selection of concepts that are further described below in the Detailed Description. This Summary is not intended to identify key or essential features of the claimed subject matter, nor is it intended to be used as an aid in limiting the scope of the claimed subject matter.

The present disclosure is based, in part, on the discovery by the inventors of novel formulation approaches for hydroximate-based compounds to enhance solubility, efficacy, safety, and the like. In particular, such approaches utilize a phosphate synthesis scheme to generate a hydroxamate-based-phosphate prodrug which exhibits enhanced solubility, efficacy and safety. The methods provided herein can be used on any hydroxamate-based compound.

Thus, in a broad aspect, the present disclosure comprises a hydroxamate-based phosphate prodrug comprising, consisting of, or consisting essentially of a hydroxamate-based compound a phosphate group.

Another aspect of the present disclosure encompasses the compounds of Formula (I), (IV) and (VI), shown herewith, pharmaceutical compositions containing those compounds, and methods of using such compounds to treat and/or prevent bacterial infections or cancers.

Hence, one aspect of the disclosure provides compounds of Formula (I):

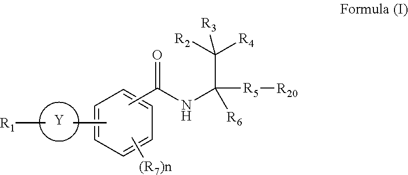

or a stereo-isomer, an enantiomer, a polymorph, a pharmaceutically acceptable salt, an ester, or a derivative thereof. In Formula (I), Y represents aryl optionally substituted with R₈, heteroaryl optionally substituted with R₈, cycloalkyl, or heterocyclyl optionally substituted with R₈;
n is an integer selected from 0, 1, 2, 3, or 4;
R₁ is hydrogen, C₁-C₆ alkyl optionally substituted with R₉, aryl optionally substituted with R₉, heteroaryl optionally substituted with R₉, or heterocyclyl optionally substituted with R₉;
R₂ is halogen, C₁-C₆ alkyl, C₁-C₆haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₃ is halogen, C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₄ is C₁-C₆ haloalkyl;
R₅ is —C₁-C₆ alkyl-, —C₁-C₆ haloalkyl-, —NH—, —NH(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₆ is hydrogen or C₁-C₆ alkyl;
each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
each R₂₀ is independently of Formula (IIa), Formula (IIb), or Formula (IIc):

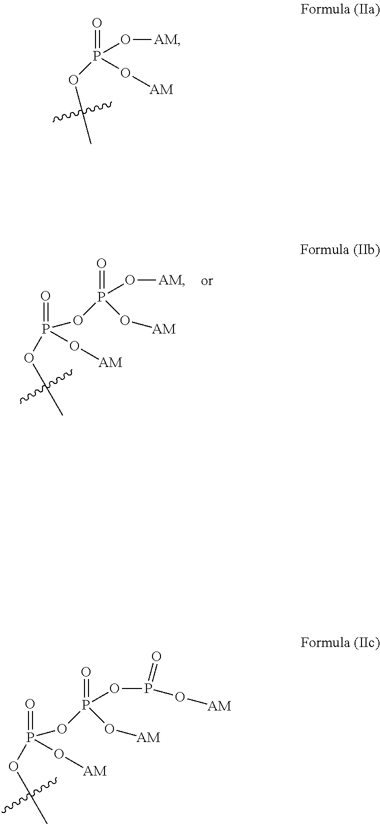

wherein each AM is independently selected from Na, Li, K, Rb, or a C₁-C₆ alkyl.

Another aspect of the present disclosure provides compounds of Formula (I), and R₂ is selected from C₁-C₆ alkyl, hydroxyl, or —O—R₂₀; R₃ is selected from C₁-C₆ alkyl, hydroxyl, or —O—R₂₀; and R₅ is selected from the group consisting of —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, and —CONH—NH—.

Yet in another aspect of Formula (I), its moiety

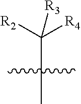

has a structure of Formula (IIIa) or Formula (IIIb):

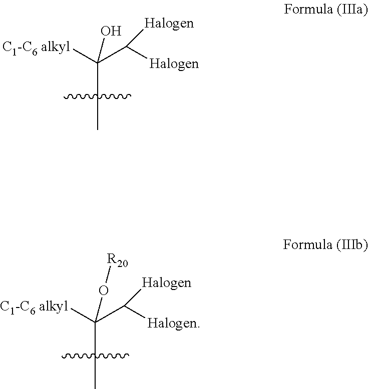

In another aspect of compounds of Formula (I), Y is selected from the group of consisting of phenyl, pyridinyl, pyrimidinyl, ethyleniminyl, cyclopropyl, pyrrolyl, furanyl, tetrahydrofuranyl, aziridinyl, pyrrolidinyl, pyrrolidonyl, thiophenyl, piperidinyl, thiazol, azulenyl, naphthyl, and imidazole, which are optionally substituted with R₈.

Another aspect of the present disclosure provides compounds of Formula (IV):

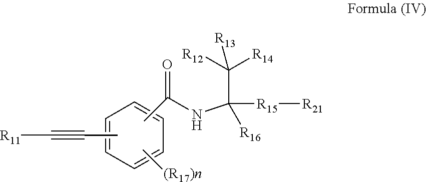

or a stereo-isomer, an enantiomer, a polymorph, a pharmaceutically acceptable salt, an ester or a derivative thereof, wherein
n is an integer selected from 0, 1, 2, 3, or 4;
R₁₁ is —C≡C—R₃₃, C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈;
wherein R₃₃ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₉, (C₃-C₈ cycloalkyl) C₁-C₆ alkyl-optionally substituted with R₁₉, a C₃-C₇ cyclyl or heterocyclyl optionally substituted with R₁₉;
R₁₂ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₃ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₄ is C₁-C₆ haloalkyl;
R₁₅ is —C₁-C₆ alkyl-, —C₁-C₆ haloalkyl-, —NH—, —NH(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₁₆ is hydrogen or C₁-C₆ alkyl;
each R₁₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₁₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —C₁-C₆ alkyl-OH, —C₁-C₆ alkyl-(C₁-C₆ alkoxy), —C₁-C₆ alkyl-NH₂, —C₁-C₆alkyl-NH—C₁-C₆ alkyl), —C₁-C₆ alkyl-N(C₁-C₆ alkyl)₂, —C₁-C₆ alkyl-NH(SO₂ C₁-C₆ alkyl), —CONH₂, —CONH(C₁-C₆ alkyl), —CON(C₁-C₆ alkyl)₂, —NH(SO₂ C₁-C₆ alkyl), —CONH—OH, —CONH—OCO(C₁-C₆ alkyl), —C(NH)NH—OH, —CONH—NH₂, —CO(C₁-C₆ alkyl), —CO₂H, —CO₂(C₁-C₆ alkyl), —C₁-C₆ alkyl-CONH₂, —C₁-C₆ alkyl-CONH(C₁-C₆ alkyl), —C₁-C₆ alkyl- CON(C₁-C₆ alkyl)₂, —C₁-C₆ alkyl-CONH—OH, —C₁-C₆ alkyl-CO(C₁-C₆ alkyl), —C₁-C₆ alkyl- CO₂H, and —C₁-C₆ alkyl-CO₂(C₁-C₆ alkyl); or two R₁₈ groups when attached to the same carbon atom form ═O;
each R₁₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —C₁-C₆ alkyl-OH, —C₁-C₆ alkyl-(C₁-C₆ alkoxy), —C₁-C₆ alkyl-NH₂, —C₁-C₆ alkyl-NH—C₁-C₆ alkyl), —C₁-C₆ alkyl-N(C₁-C₆ alkyl)₂, —C₁-C₆ alkyl-NH(SO₂ C₁-C₆ alkyl), —CONH₂, —CON(C₁-C₆ alkyl), —CON(C₁-C₆ alkyl)₂, —NH(SO₂ C₁-C₆ alkyl), —CONH—OH, —CONH—OCO(C₁-C₆ alkyl), —C(NH)NH—OH, —CONH—NH₂, —CO(C₁-C₆ alkyl), —CO₂H, —CO₂(C₁-C₆ alkyl), —C₁-C₆ alkyl-CONH₂, —C₁-C₆ alkyl-CON(C₁-C₆ alkyl), —C₁-C₆ alkyl- CON(C₁-C₆ alkyl)₂, —C₁-C₆ alkyl-CONH—OH, —C₁-C₆ alkyl-CO(C₁-C₆ alkyl), —C₁-C₆ alkyl- CO₂H, and —C₁-C₆ alkyl-CO₂(C₁-C₆ alkyl); or two R₁₉ groups when attached to the same carbon atom form ═O; and
each R₂₁ is independent of Formula (IIa), Formula (IIb), or Formula (IIc):

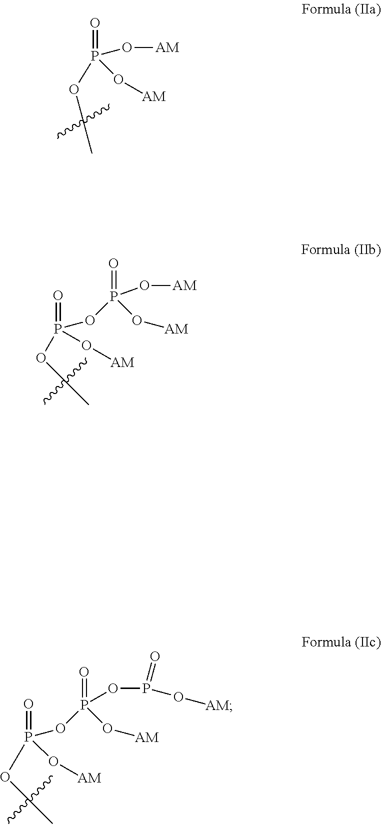

and each AM is independently selected from Na, Li, K, Rb, or a C₁-C₆ alkyl.

In another aspect of the compounds of Formula (IV), R₁₂ is selected from C₁-C₆ alkyl, hydroxyl, or —O—R₂₁; R₁₃ is selected from C₁-C₆ alkyl, hydroxyl, or —O—R₂₁; and R₁₅ is selected from the group consisting of —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, and —CONH—NH—.

In yet another aspect of Formula (IV), its moiety of

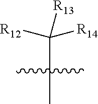

is of Formula (Va) or Formula (Vb):

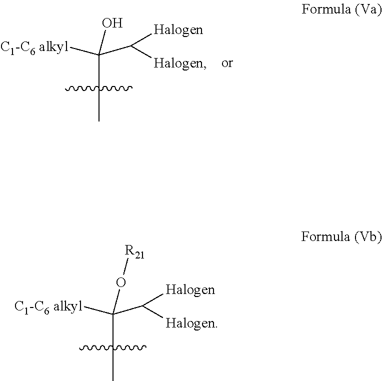

In yet another of the compounds of Formula (IV), R₁₁ is selected from —C≡C—R₃₃, or C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈; and R₃₃ is —C₁-C₆ alkyl-CO₂H, C₃-C₈ cycloalkyl optionally substituted with R₁₉, (C₃-C₈ cycloalkyl) C₁-C₆ alkyl-, optionally substituted with R₁₉, a C₃-C₇ cyclyl or heterocyclyl optionally substituted with R₁₉.

In another aspect, the present disclosure provides compounds of Formula (VI):

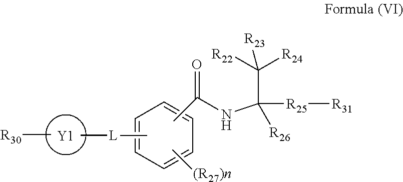

or a stereo-isomer, an enantiomer, a polymorph, a pharmaceutically acceptable salt, an ester, or a derivative thereof, wherein
L represents a linker, wherein L is absent or is selected from the group consisting of —C≡C—C≡C—, or —C≡C—R₃₂, wherein R₃₂ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₂₉, (C₃-C₈ cycloalkyl) C₁-C₆ alkyl- optionally substituted with R₂₉, a C₃-C₇ cyclyl and heterocyclyl optionally substituted with R₂₉;
Y₁ represents aryl optionally substituted with R₂₈, heteroaryl optionally substituted with R₂₈, cycloalkyl, or heterocyclyl optionally substituted with R₂₈;
n is an integer selected from 0, 1, 2, 3, or 4;
R₃₀ is hydrogen, C₁-C₆ alkyl optionally substituted with R₂₉, aryl optionally substituted with R₂₉, heteroaryl optionally substituted with R₂₉, or heterocyclyl optionally substituted with R₂₉;
R₂₂ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₃ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₄ is C₁-C₆ haloalkyl;
R₂₅ is —C₁-C₆ alkyl-, —C₁-C₆ haloalkyl-, —NH—, —NH(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₂₆ is hydrogen or C₁-C₆ alkyl;
each R₂₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₂₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl). —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
each R₃₁ is independently a compound of Formula (IIa), Formula (IIb), or Formula (IIc):

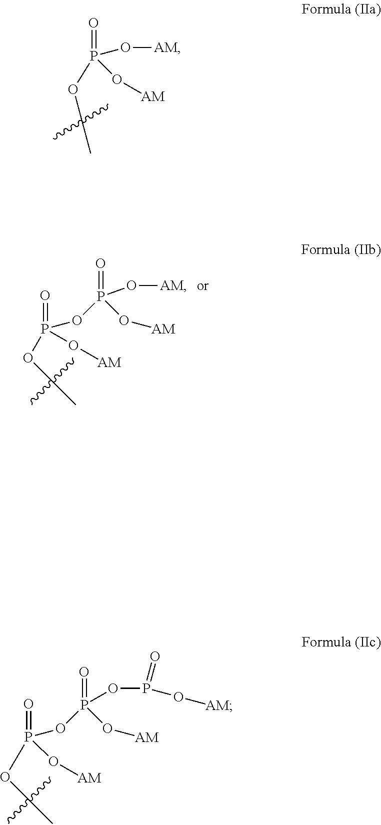

and each AM is independently selected from Na, Li, K, Rb, or a C₁-C₆ alkyl.

In one aspect of compounds of the Formula (VI), R₂₂ is selected from C₁-C₆ alkyl, hydroxyl, or —O—R₃₁; R₂₃ is selected from C₁-C₆ alkyl, hydroxyl, or —O—R₃₁; And R₂₅ is selected from the group consisting of —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, and —CONH—NH—.

In another aspect of compounds of the Formula (VI), its moiety

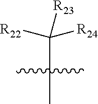

is of Formula (VIIa) or Formula (VIIb):

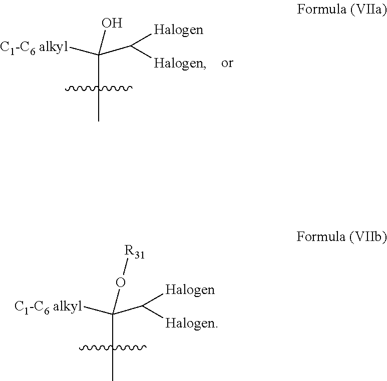

In yet another aspect of compounds of Formula (VI), Y₁ is selected from the group consisting of phenyl, pyridinyl, pyrimidinyl, ethyleniminyl, cyclopropyl, pyrrolyl, furanyl, tetrahydrofuranyl, aziridinyl, pyrrolidinyl, pyrrolidonyl, thiophenyl, piperidinyl, thiazol, azulenyl, naphthyl, and imidazole, which are optionally substituted with R₂₈.

Another aspect of the present invention is of a phosphate prodrug, and in such prodrug, the AM as defined in Formula (IIa) to (IIc) is either sodium or methyl.

In another aspect, the present disclosure provides a compound having the formulas:

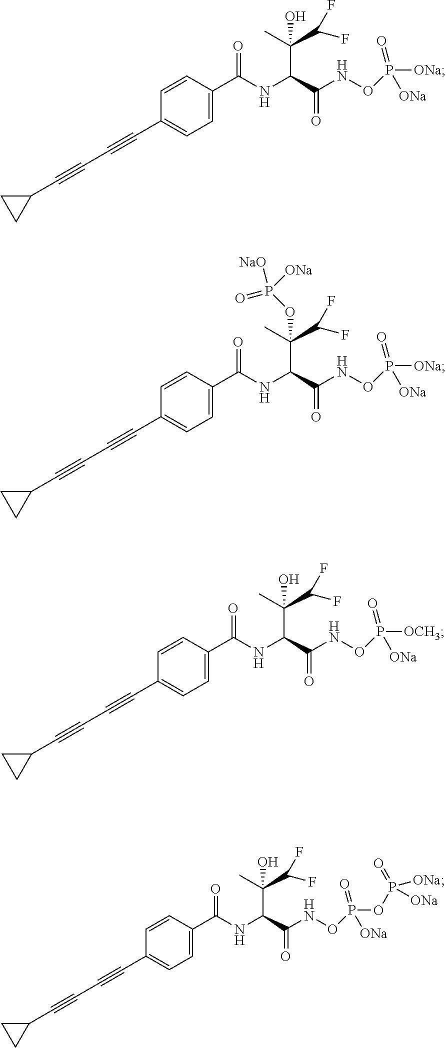

or a polymorph, a pharmaceutically acceptable salt, an ester or a derivative thereof.

In one aspect, the present disclosure provides a compound of the following formulas:

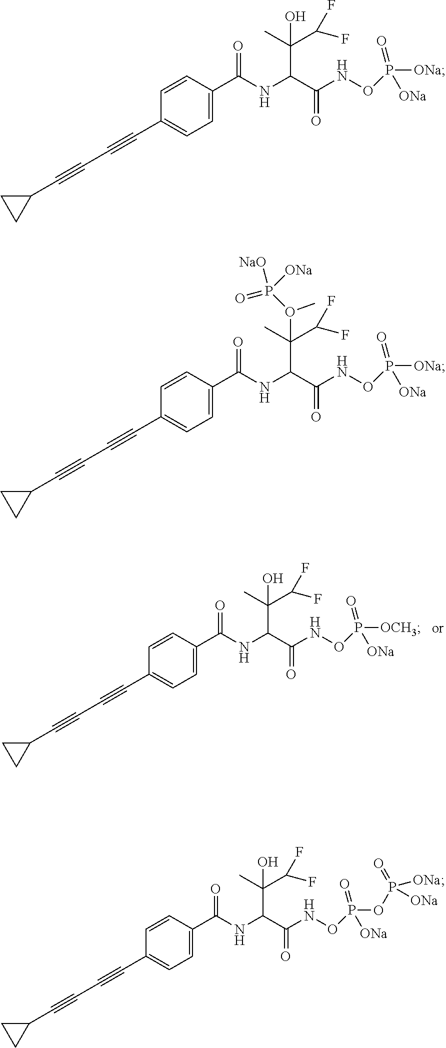

or a stereo-isomer, an enantiomer, a polymorph, a pharmaceutically acceptable salt, an ester or a derivative thereof.

In another aspect, the compound of the present disclosure may comprise one of the following compounds:

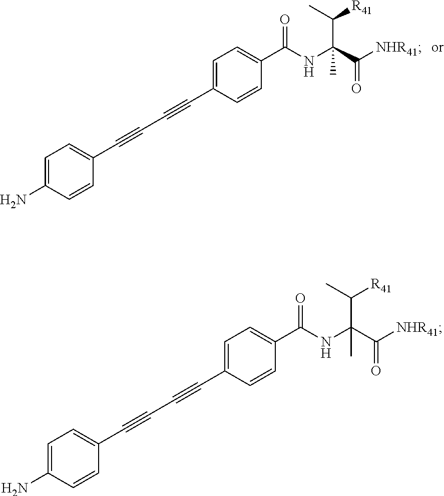

wherein each R₄₁ is independently selected from —OH, Formula (IIa). Formula (IIb), or Formula (IIc),

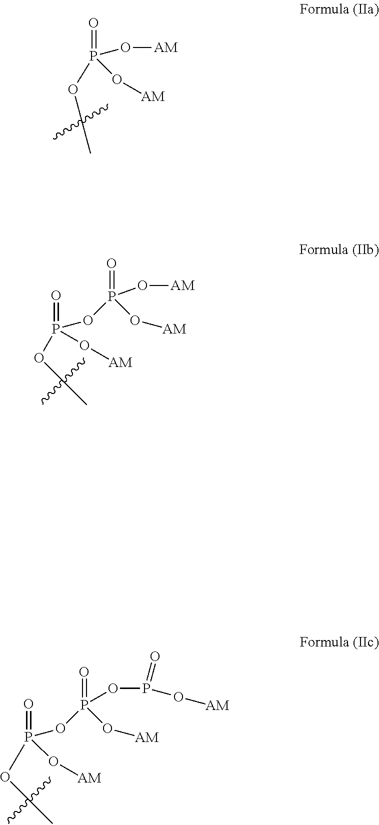

and wherein each AM is independently selected from Na, Li, K, Rb, or a C₁-C₆ alkyl.

In another aspect, the present disclosure provides mono-phosphate, di-phosphate (i.e. pyrophosphate) or tri-phosphate prodrug of a compound selected from the following formulas, and the phosphate is formed with a hydroxyl group or a hydroxamate oxo group of the compound:

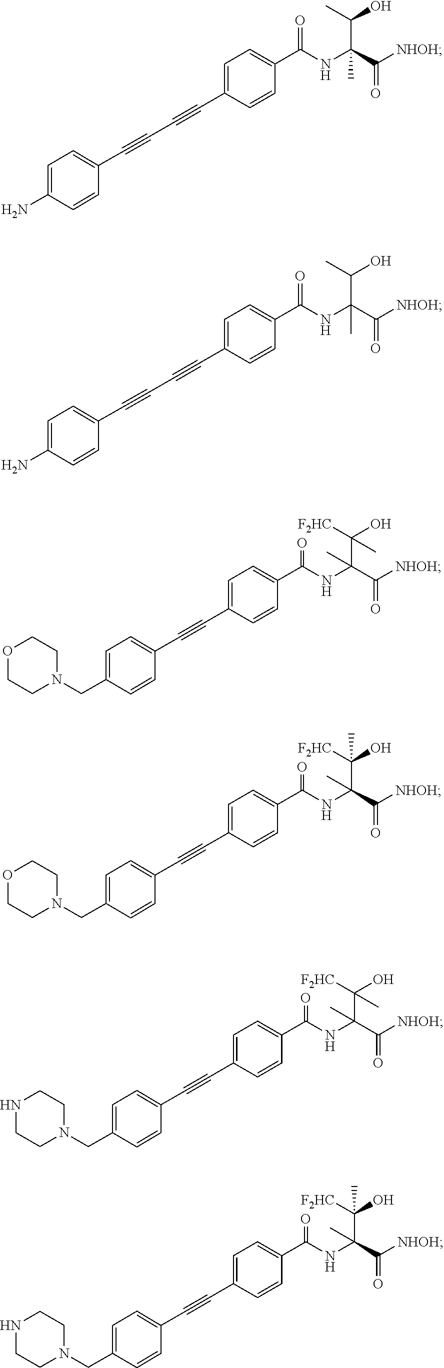

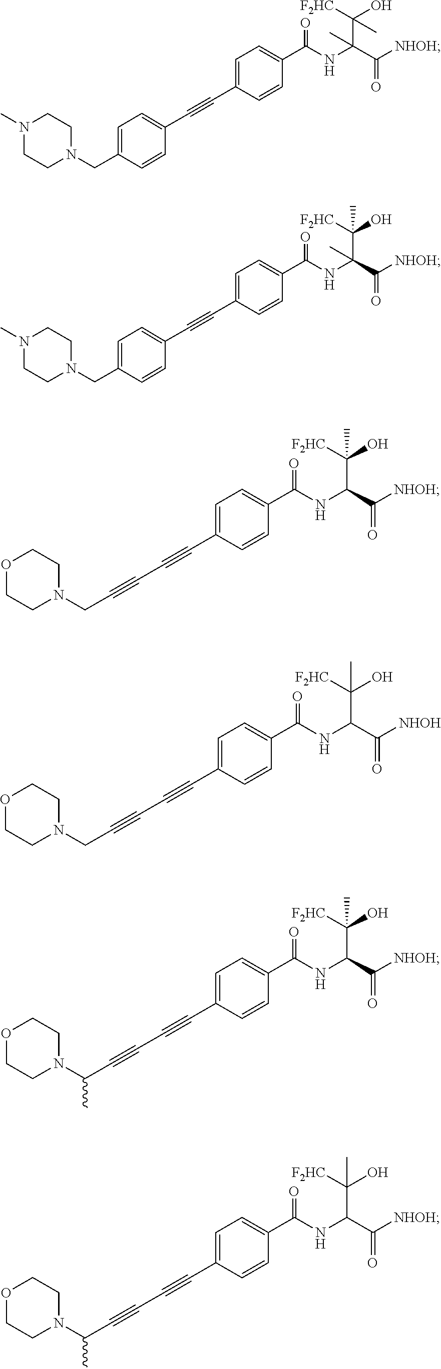

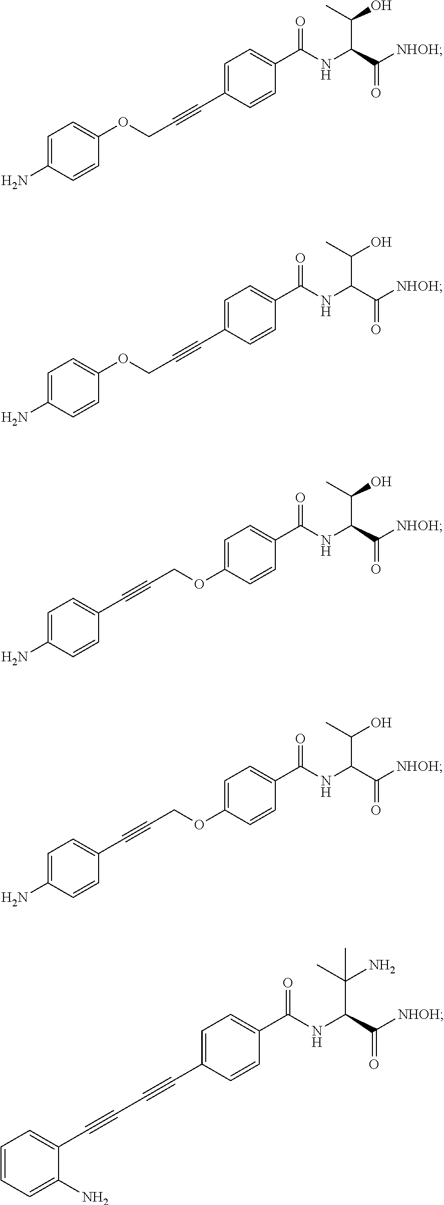

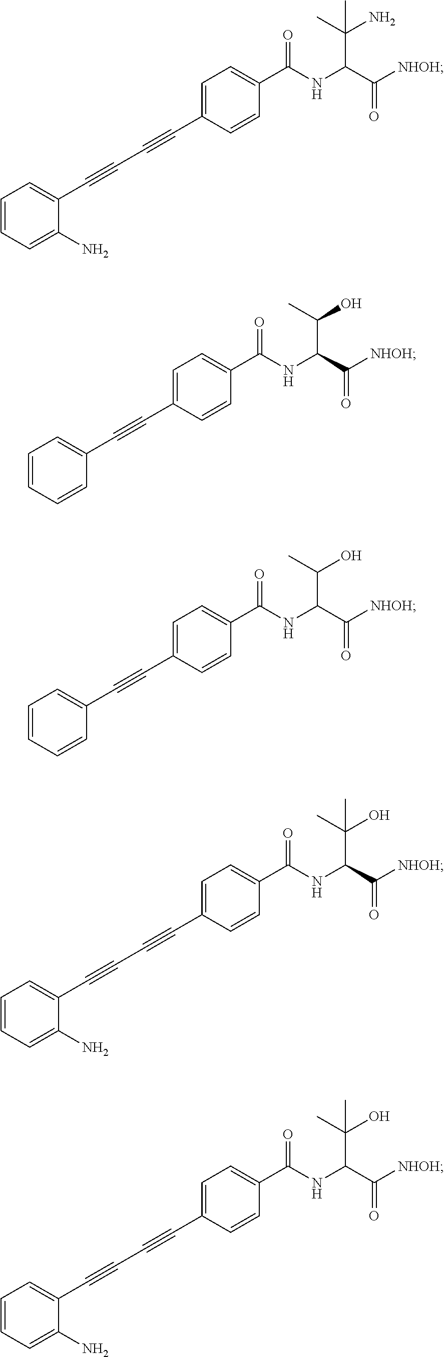

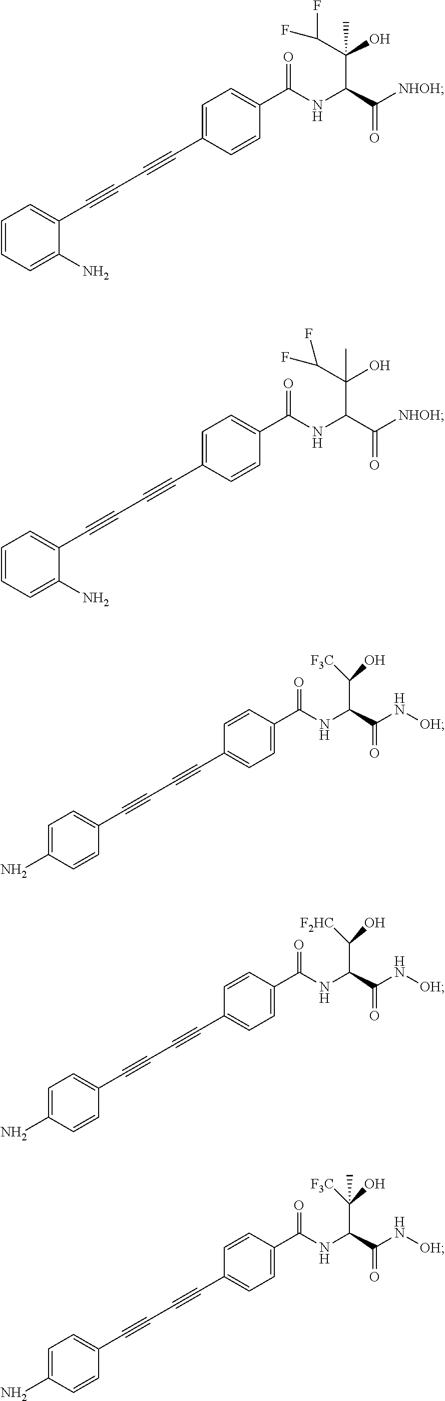

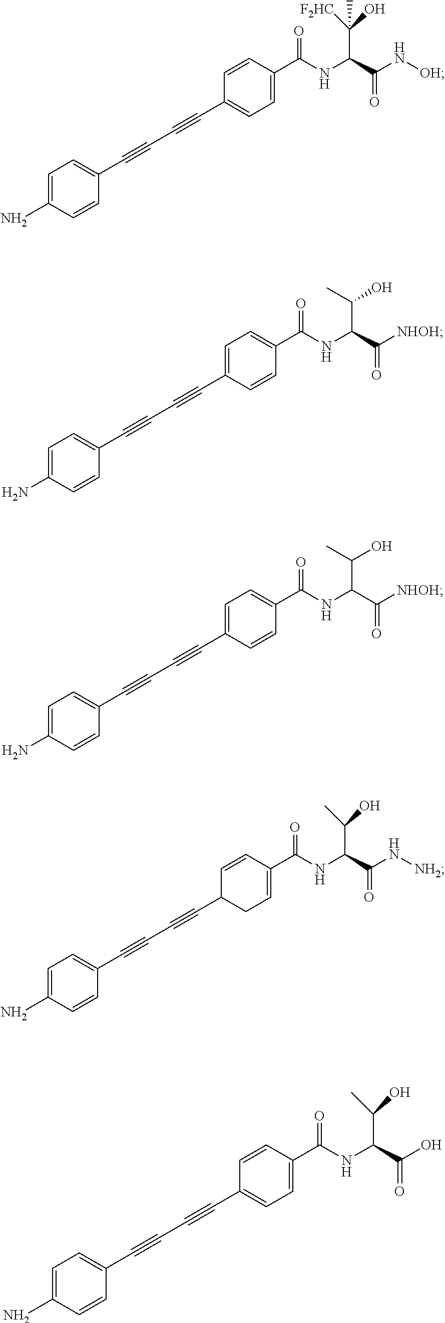

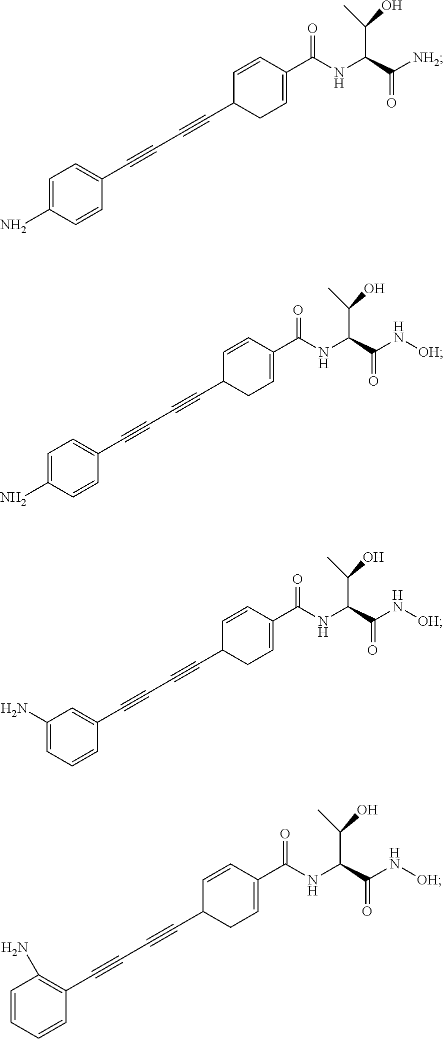 [SMILES: C[C@@H](O)[C@H](NC(=O)C1=CCC(C#CC#Cc2ccc(N)cc2)C=C1)C(N)=O ; C[C@@H](O)[C@H](NC(=O)C1=CCC(C#CC#Cc2ccc(N)cc2)C=C1)C(=O)NO ; C[C@@H](O)[C@H](NC(=O)C1=CCC(C#CC#Cc2cccc(N)c2)C=C1)C(=O)NO ; C[C@@H](O)[C@H](NC(=O)C1=CCC(C#CC#Cc2ccccc2N)C=C1)C(=O)NO]

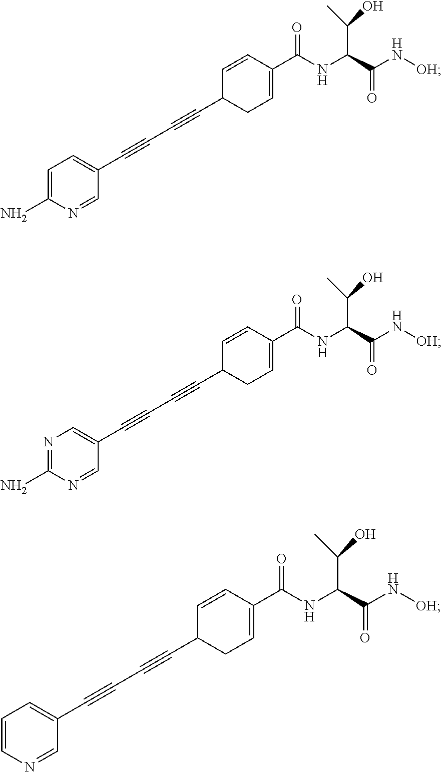 [SMILES: C[C@@H](O)[C@H](NC(=O)C1=CCC(C#CC#Cc2ccc(N)nc2)C=C1)C(=O)NO ; C[C@@H](O)[C@H](NC(=O)C1=CCC(C#CC#Cc2cnc(N)nc2)C=C1)C(=O)NO ; C[C@@H](O)[C@H](NC(=O)C1=CCC(C#CC#Cc2cccnc2)C=C1)C(=O)NO]

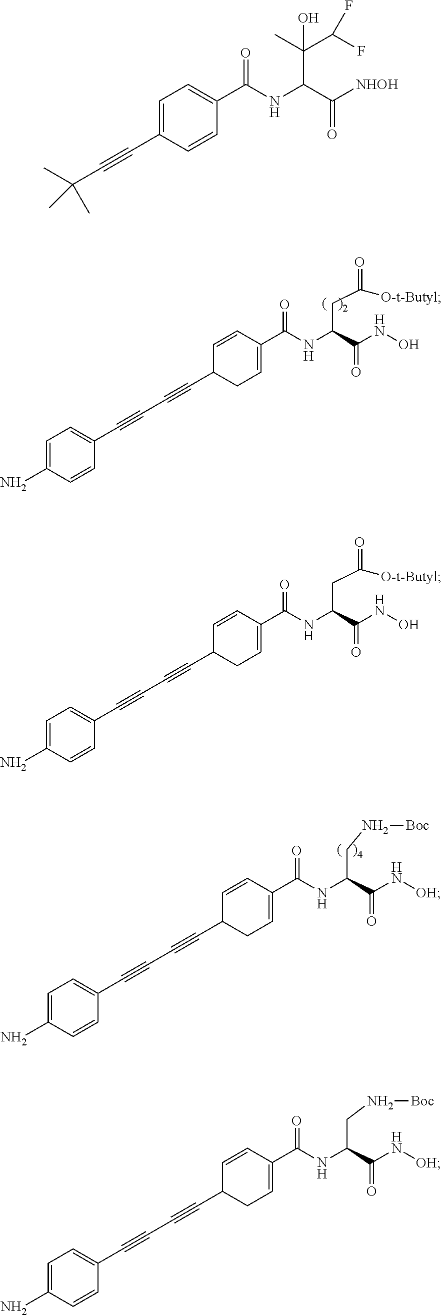

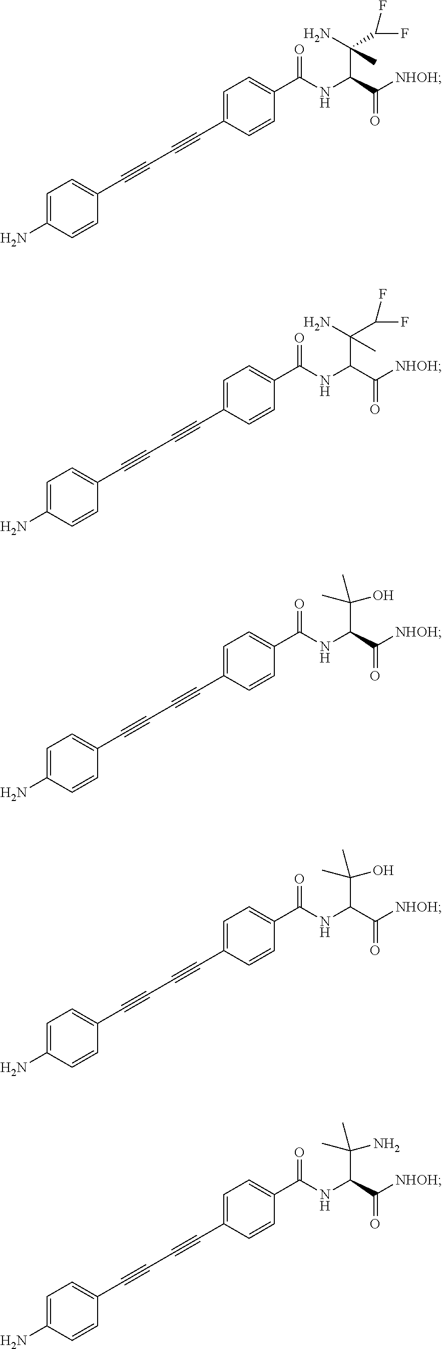

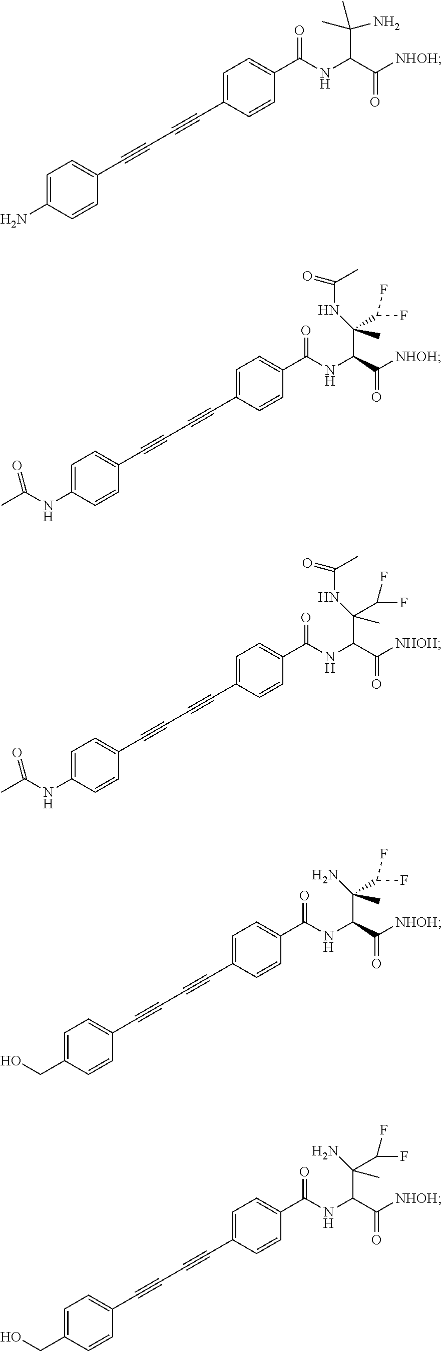

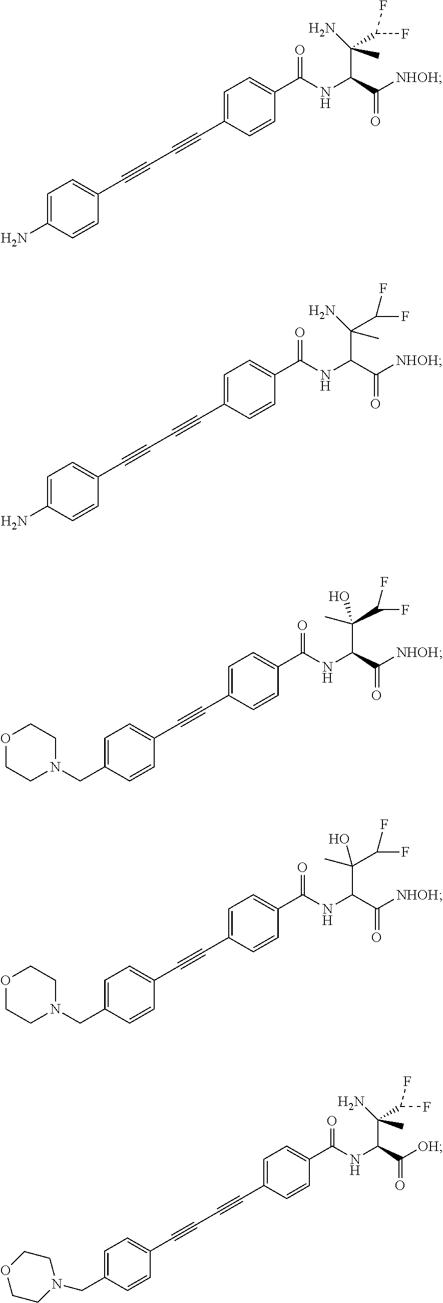

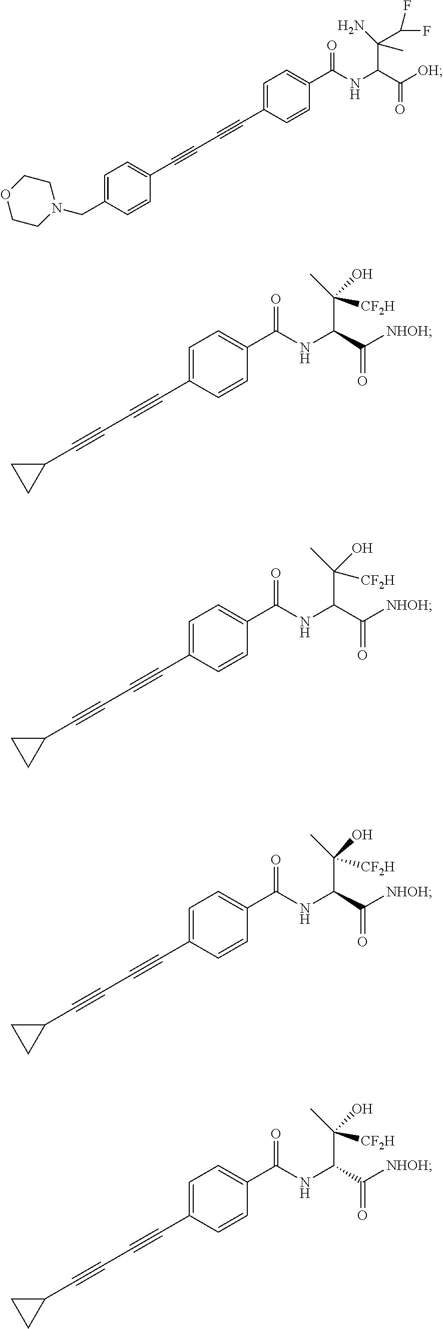

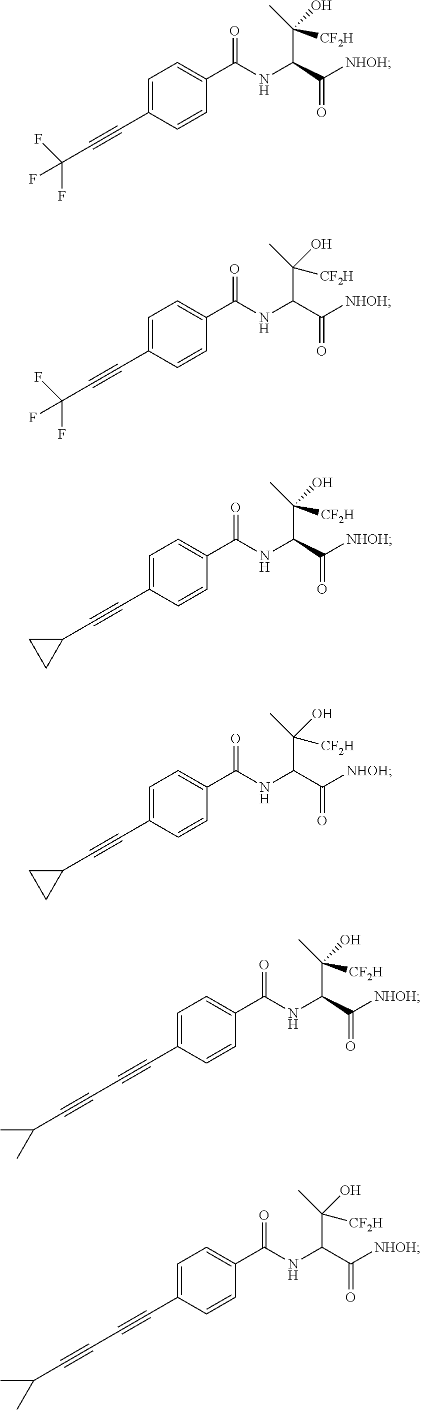

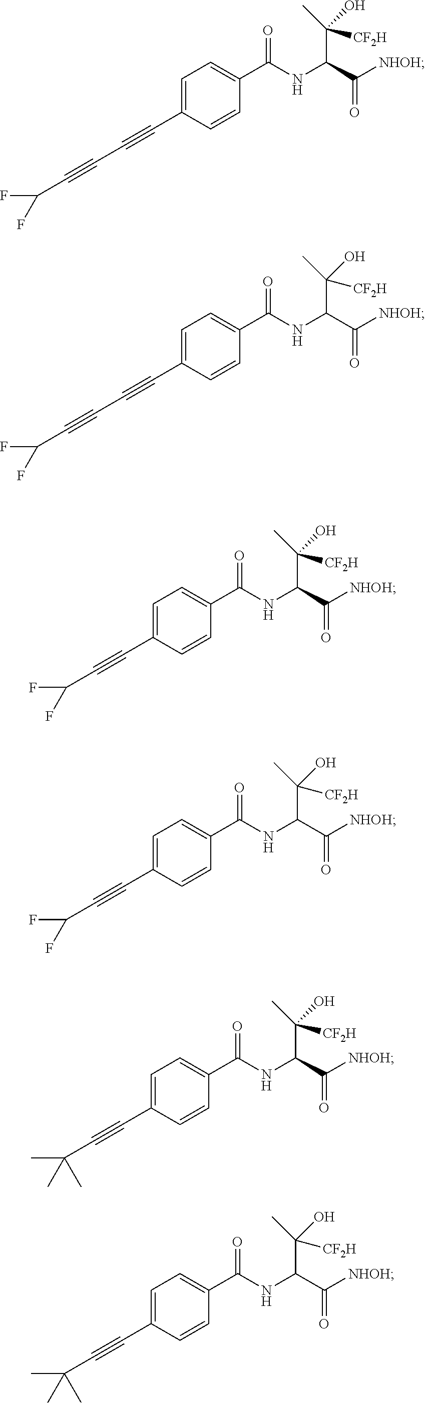

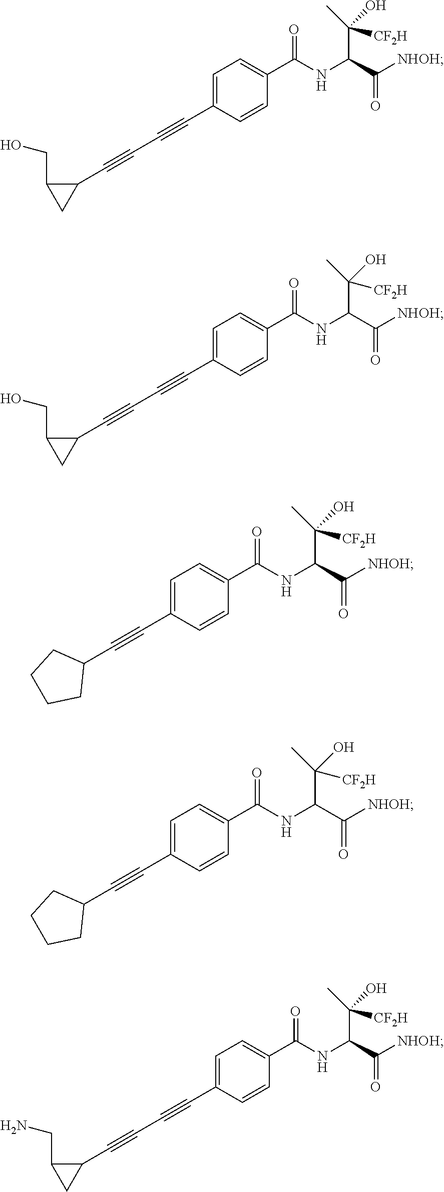

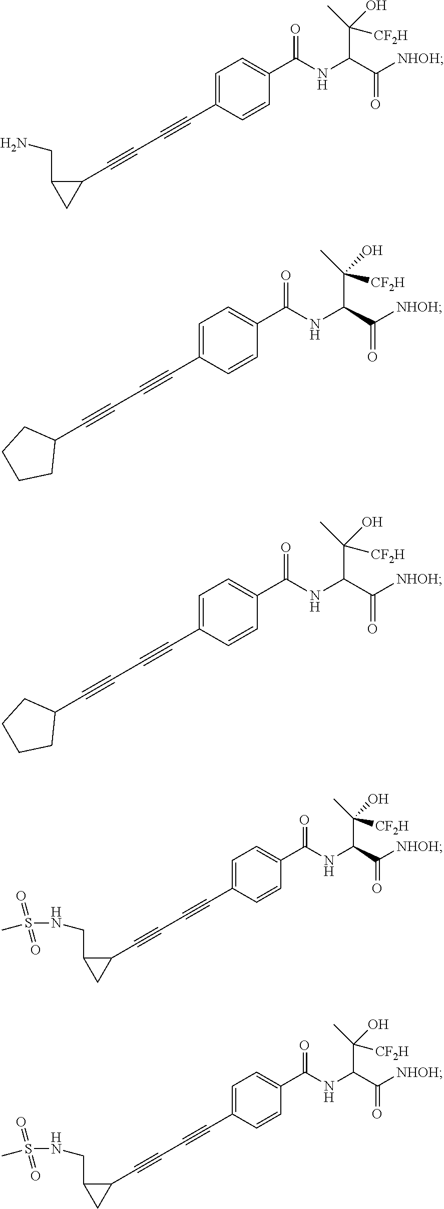

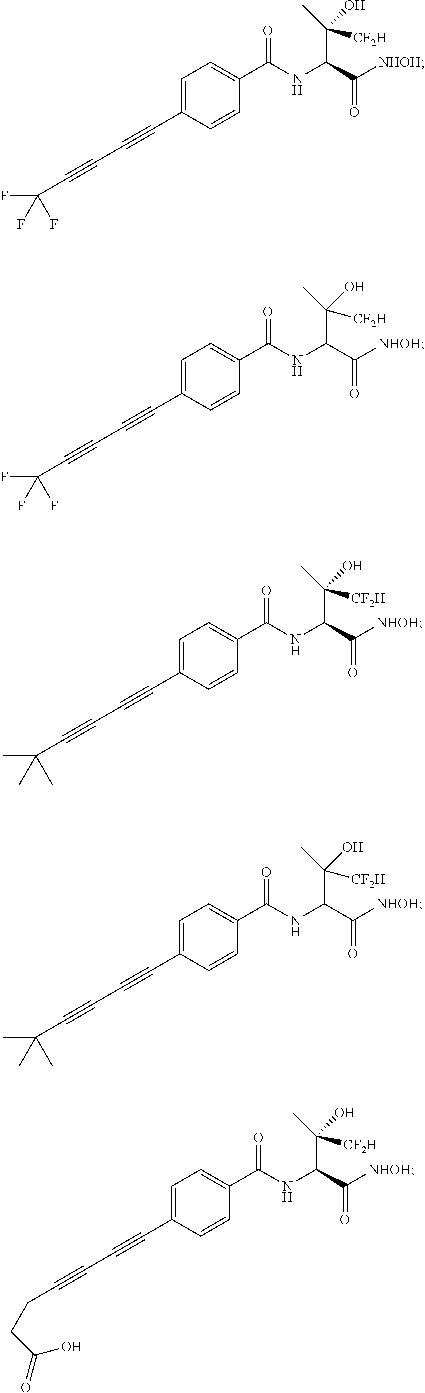

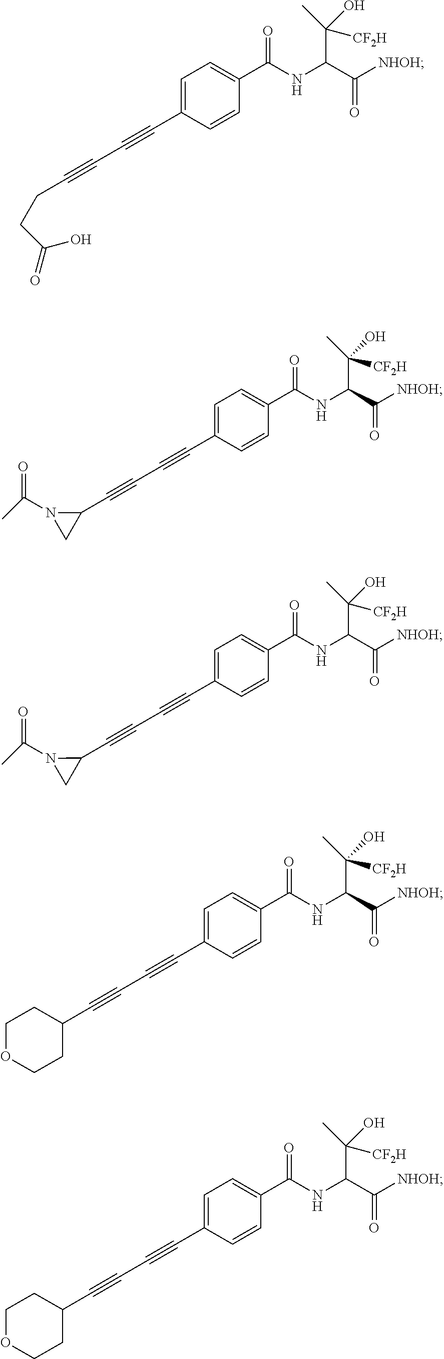

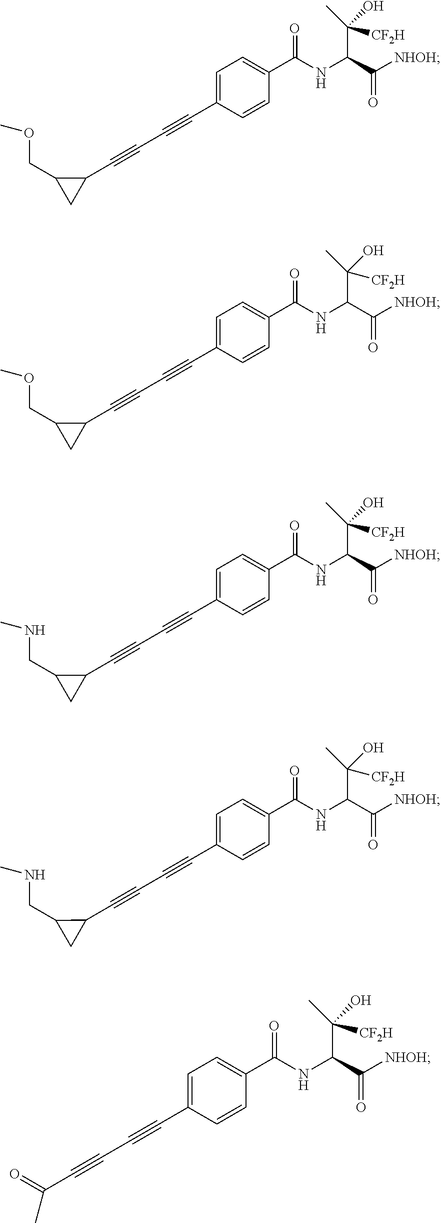

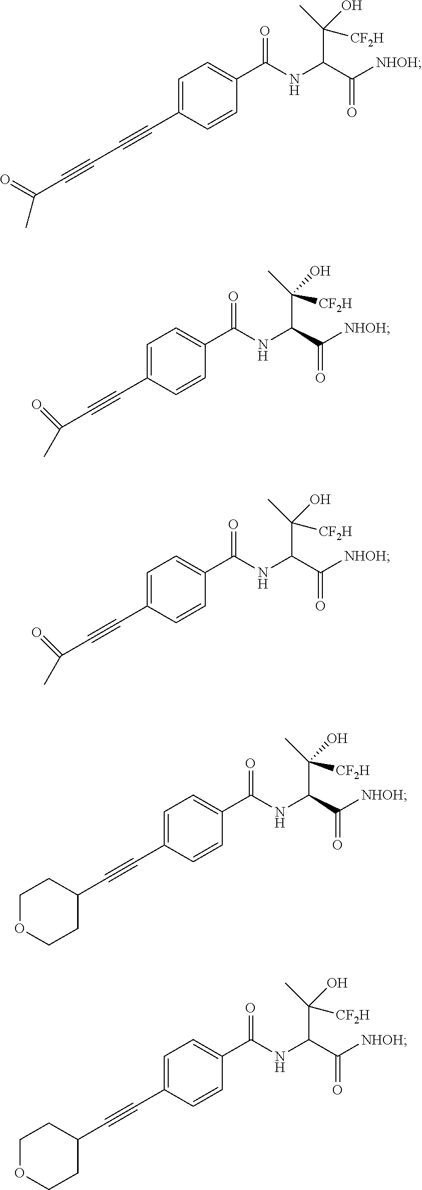

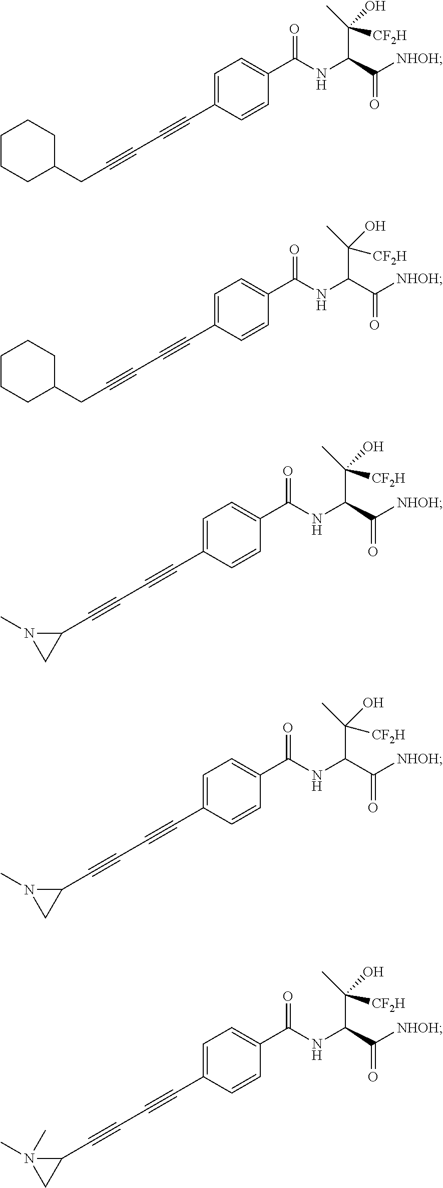

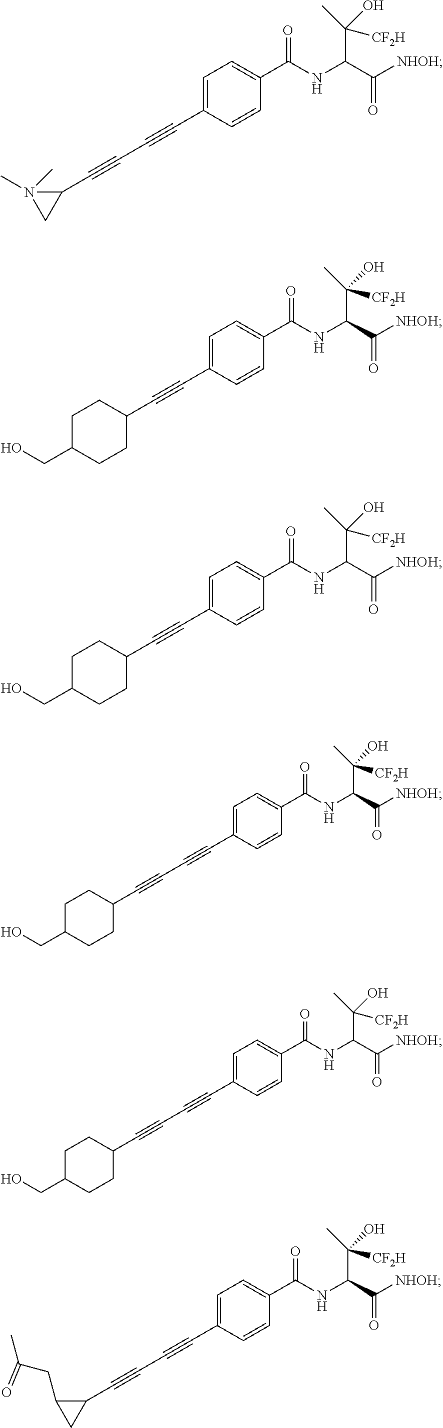

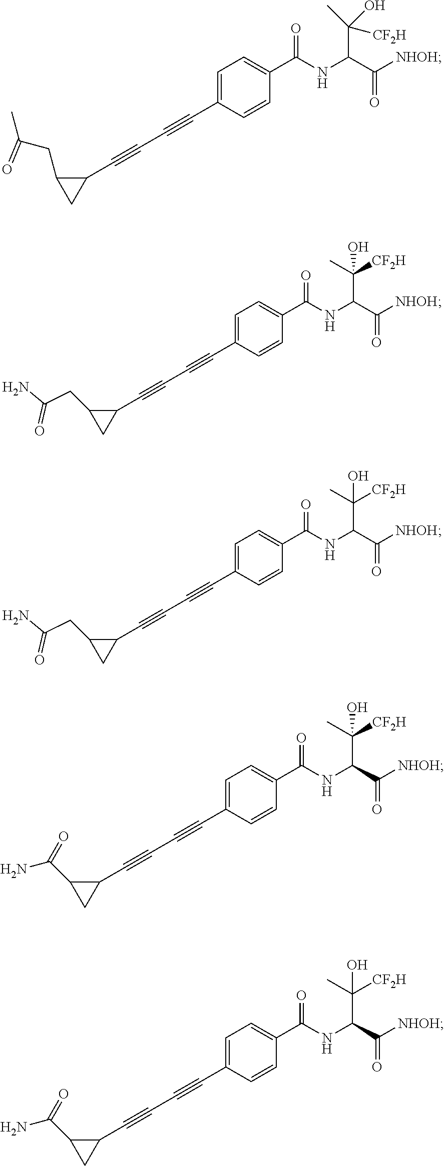

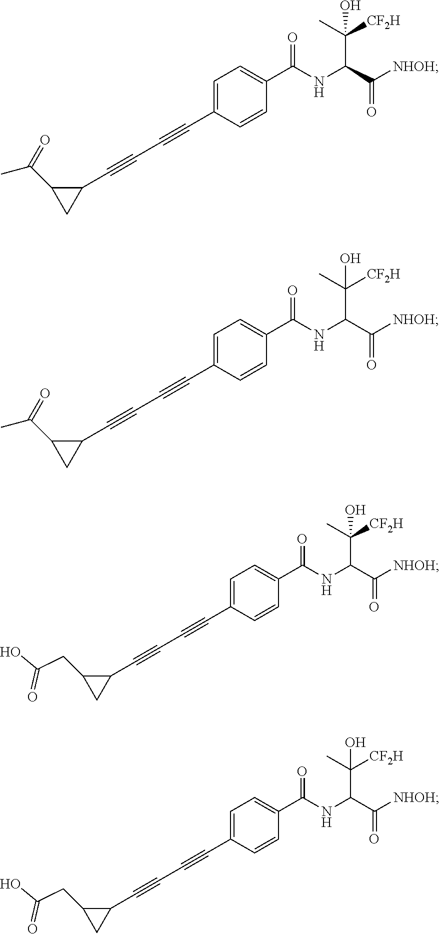

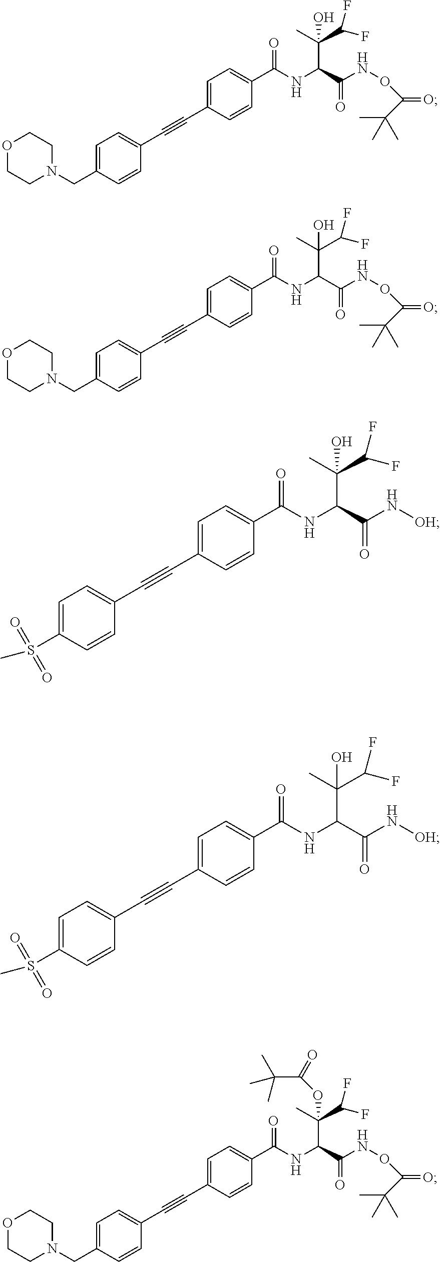 [SMILES: CC(C)(C)C(=O)ONC(=O)[C@@H](NC(=O)c1ccc(C#Cc2ccc(CN3CCOCC3)cc2)cc1)[C@](C)(O)C(F)F ; CC(C)(C)C(=O)ONC(=O)C(NC(=O)c1ccc(C#Cc2ccc(CN3CCOCC3)cc2)cc1)C(C)(O)C(F)F ; C[C@@](O)(C(F)F)[C@H](NC(=O)c1ccc(C#Cc2ccc(S(C)(=O)=O)cc2)cc1)C(=O)NO ; CC(O)(C(F)F)C(NC(=O)c1ccc(C#Cc2ccc(S(C)(=O)=O)cc2)cc1)C(=O)NO ; CC(C)(C)C(=O)ONC(=O)[C@@H](NC(=O)c1ccc(C#Cc2ccc(CN3CCOCC3)cc2)cc1)[C@](C)(OC(=O)C(C)(C)C)C(F)F]

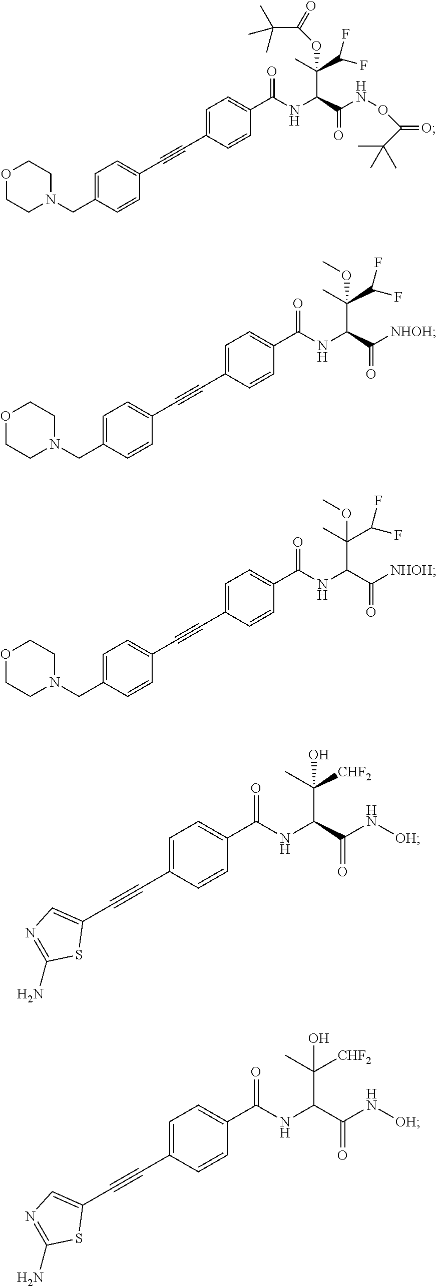

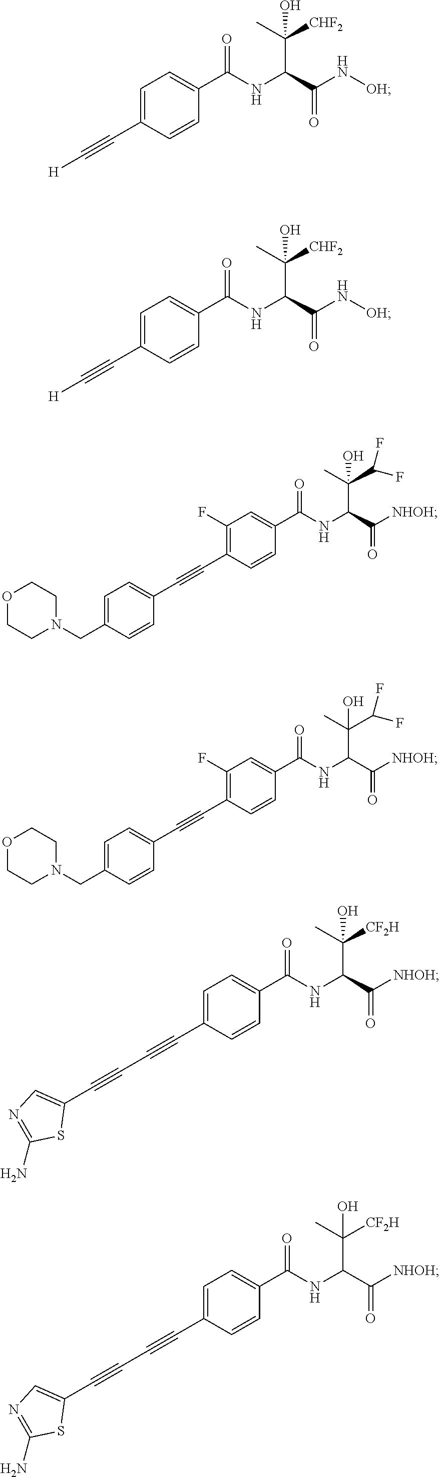

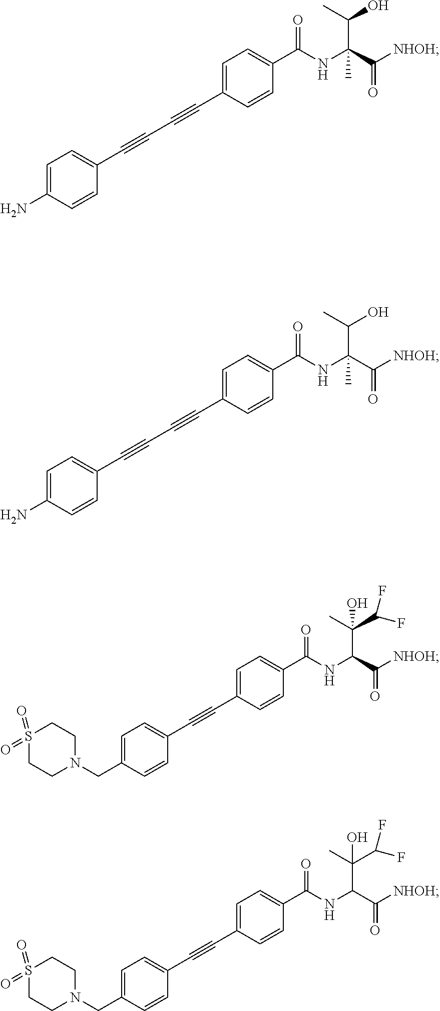

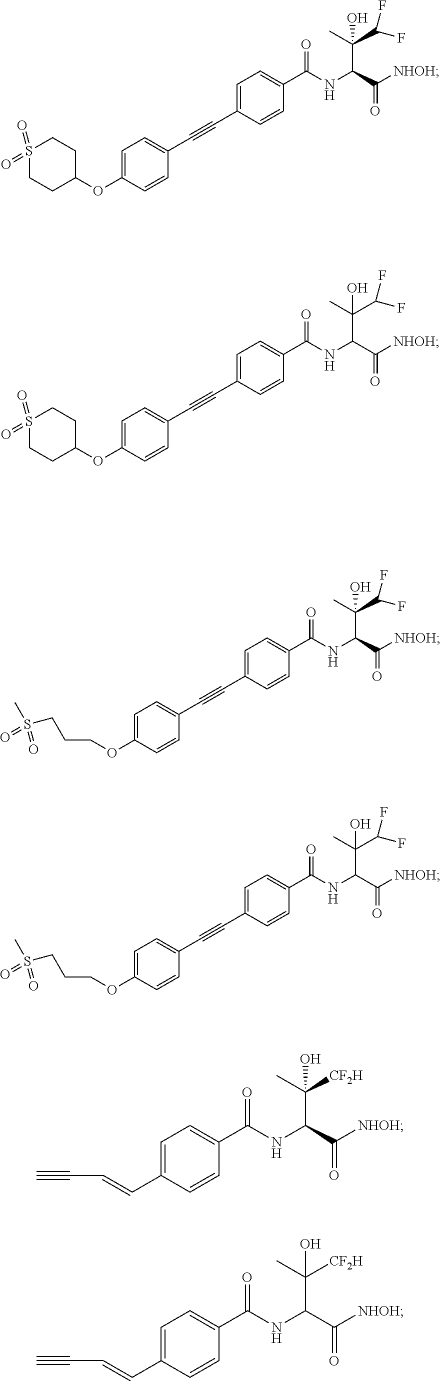

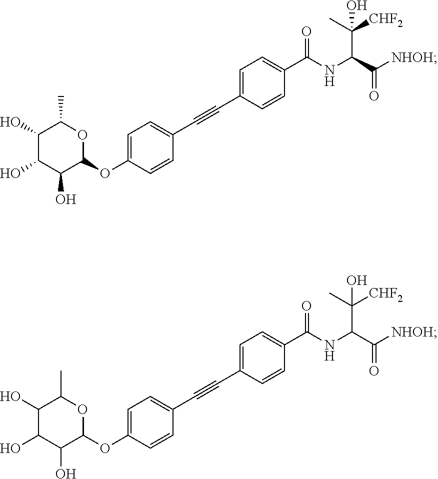

or a stereo-isomer, an enantiomer, a polymorph, a pharmaceutically acceptable salt, an ester, or a derivative thereof.

The present disclosure also provides pharmaceutical compositions comprising a compound of Formula I, Formula IV, or Formula VI, or stereo-isomer, an enantiomer, a polymorph, a pharmaceutically acceptable salt thereof and at least one pharmaceutically acceptable carrier, solvent, adjuvant or diluent.

The disclosure also provides methods for inhibiting UDP-3-O—(R-3-hydroxydecanoyl)-N-acetylglucosamine deacetylase (LpxC), and methods of treating Gram-negative bacterial infections.

In one aspect, the method comprises administering to a subject in need of such treatment an effective amount of one or more compounds or composition of the present disclosure, such as compounds of Formula (I), Formula (IV), or Formula (VI). In one aspect, the Gram-negative bacteria is selected from the group consisting of Pseudomonas aeruginosa, Stenotrophomonas maltophilia, Burkholderia cepacia, Alcaligenes xylosoxidans, Acinetobacter, Enterobacteriaceae, Haemophilus, Neisseria, Francisella tularensis, Yersinia pestis, Burkholderia pseudomallei, Burkholderia mallei, Rickettsia prowazekii, Coxiella burnetti, Campylobacter jejuni, Shigella, Moraxella catarrhalis, Chlamydia trachomatis, and combinations thereof. In one aspect, the Gram-negative bacteria comprises Neisseria gonorrhoeae. In another aspect, the Gram-negative bacteria comprises Acinetobacter Baumannii. In yet another aspect, the Gram-negative bacteria comprises an Enterobacteriacacae. In one aspect, the Enterobacteriaceae is selected from the group consisting of Serratia, Proteus, Klebsiella, Enterobacter, Citrobacter, Salmonella, Providencia, Morganella, Cedecea, Edwardsiella, Escherichia coli, Enterobacter cloacae, Enterobacter aerogenes, and combinations thereof.

The disclosure further provides inhibiting a deacetylase enzyme in Gram-negative bacteria by contacting the bacteria with an effective amount of the compounds or the composition of the present disclosure. In one aspect, the deacetylase enzyme comprises LpxC.

The disclosure further provides methods of treating a cancer in a subject, the method comprises administering to a subject in need of such treatment an effective amount of one or more compounds of the present disclosure. In one aspect, the cancer is selected from the group consisting of breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma, peripheral neuroepithelioma, and combinations there.

The present disclosure further provides methods of administering to the subject one or more additional therapeutic agents. In one aspect, the one or more additional therapeutic agent is administered prior to, concurrently, and/or after administering the compound or the composition of the present disclosure.

The present disclosure further provides a kit. Such kit comprises (1) a container holding the compound and/or composition of the present disclosure, and (2) an instruction for use. In one aspect, the instruction for use comprises steps in administering the compound or the composition of the present disclosure to a subject in need thereof. In another aspect, the subject is a human subject having or is suspect of having a bacterial infection or a cancer.

In one aspect, the present disclosure provides use of one or more compounds or composition of the present disclosure, such as compounds of Formula (I). Formula (IV), or Formula (VI) in treating bacterial infection or cancer. In one aspect, the Gram-negative bacteria is selected from the group consisting of Pseudomonas aeruginosa, Stenotrophomonas maltophilia, Burkholderia cepacia, Alcaligenes xylosoxidans, Acinetobacter, Enterobacteriaceae, Haemophilus, Neisseria, Francisella tularensis, Yersinia pestis, Burkholderia pseudomallei, Burkholderia mallei, Rickettsia prowazekii, Coxiella burnetti, Campylobacter jejuni, Shigella, Moraxella catarrhalis, Chlamydia trachomatis, and combinations thereof. In one aspect, the Gram-negative bacteria comprises Neisseria gonorrhoeae. In another aspect, the Gram-negative bacteria comprises Acinetobacter Baumannii. In yet another aspect, the Gram-negative bacteria comprises an Enterobacteriacacae. In one aspect, the Enterobacteriaceae is selected from the group consisting of Serratia, Proteus, Klebsiella, Enterobacter, Citrobacter, Salmonella, Providencia, Morganella, Cedecea, Edwardsiella, Escherichia coli, Enterobacter cloacae, Enterobacter aerogenes, and combinations thereof. In one aspect, the cancer comprises breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma, peripheral neuroepithelioma, and combinations thereof.

In yet another aspect, the present disclosure provides use of the compounds or compositions in the manufacture of a medicament for inhibiting a deacetylase LpxC in a subject in need thereof. In one aspect, the LpxC overexpression manifests a bacterial infection or a cancer. In one aspect, the bacterial infection is caused by Pseudomonas aeruginosa, Stenotrophomonas maltophilia, Burkholderia cepacia, Alcaligenes xylosoxidans, Acinetobacter, Enterobacteriaceae, Haemophilus, Neisseria, Francisella tularensis, Yersinia pestis, Burkholderia pseudomallei, Burkholderia mallei, Rickettsia prowazekii, Coxiella burnetti, Campylobacter jejuni, Shigella, Moraxella catarrhalis, Chlamydia trachomatis, and combinations thereof. In another aspect, the cancer comprises breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma, peripheral neuroepithelioma, and combinations thereof.

In another aspect, the present disclosure provides a method of synthesizing the compounds of the present disclosure. In one aspect, the method of synthesis may comprise:
(a) contacting a compound of Formula (I′)

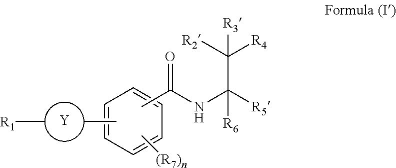

with a phosphate salt of the formula

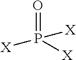

in the presence of a base catalyst to form a reaction mixture; and
(b) contacting the reaction mixture with an alkali metal hydroxide to form a compound of Formula (I):

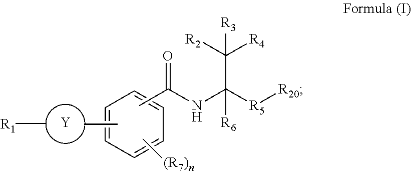

wherein
   X is a halogen;
   Y represents aryl optionally substituted with R₈, heteroaryl optionally substituted with R₈, cycloalkyl or heterocyclyl optionally substituted with R₈;
   n is an integer selected from 0, 1, 2, 3, or 4;
   R₁ is hydrogen, C₁-C₆ alkyl optionally substituted with R₉, aryl optionally substituted with R₉, heteroaryl optionally substituted with R₉, or heterocyclyl optionally substituted with R₉;
   R₂ is halogen, C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
   R₂′ is halogen, C₁-C₆ alkyl, C₁-C₆haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
   R₃ is halogen, C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
   R₃′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
   R₄ is C₁-C₆ haloalkyl;
   R₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
   R₅′ is —C₁-C₆ alkyl, —C₁-C₆ haloalkyl, —NH₂, —NH(C₁-C₆ alkyl), —NH(C₁-C₆ alkyl), —N(C₁-C₆ alkyl)₂, —OH, —C₁-C₆ alkoxy, —C₁-C₆ haloalkoxy, —CONH₂, —CONH(C₁-C₆ alkyl), —CON(C₁-C₆ alkyl), —CON(C₁-C₆ alkyl)₂, —CONH—OH, —CON(C₁-C₆ alkyl)-OH, —CONH₂, —OCO(C₁-C₆ alkyl), —O—CH(C₁-C₆ alkyl)₂, —CONH—NH₂, —CO₂H, and —CO₂(C₁-C₆ alkyl);
   R₆ is hydrogen or C₁-C₆ alkyl;
   each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
   each R₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
   each R₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
   each R₂₀ is independently of Formula (IIa), Formula (IIb), or Formula (IIc):

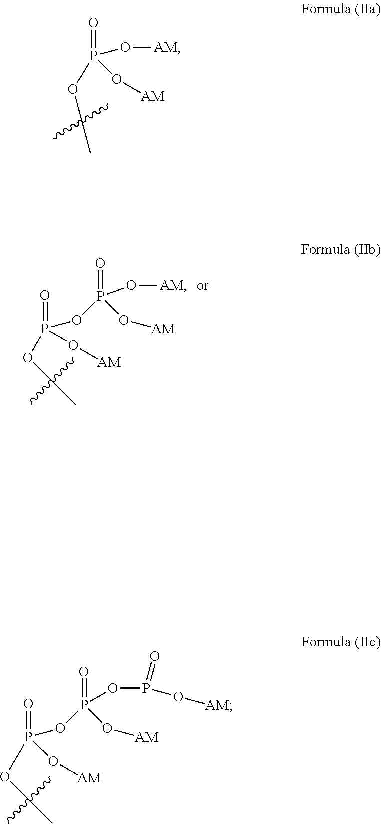

each AM is independently selected from Na, Li, K, Rb, or a C₁-C₆ alkyl. In some embodiments, X is Cl.

The present disclosure provides method of synthesis of compound of Formula (IV). In one aspect, the method comprises the steps of
(a) contacting a compound of Formula (IV′)

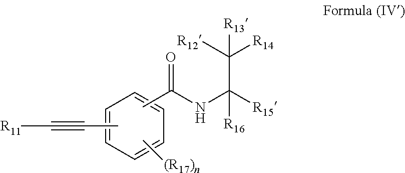

with a phosphate salt of the formula

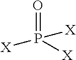

in the presence of a base catalyst to form a reaction mixture; and
(b) contacting the reaction mixture with an alkali metal hydroxide to form a compound of

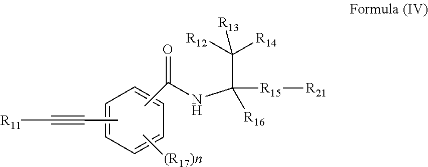

wherein
X is a halogen;
n is an integer selected from 0, 1, 2, 3, or 4;
R₁₁ is —C≡C—R₃₃, C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈;
wherein R₃₃ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₉, (C₃-C₈ cycloalkyl) C₁-C₆ alkyl-optionally substituted with R₁₉, a C₃-C₇ cyclyl or heterocyclyl optionally substituted with R₁₉;
R₁₂ is halogen, C₁-C₆ alkyl, C₁-C₆haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₂′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₃ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₃′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₄ is C₁-C₆ haloalkyl;
R₁₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₁₅′ is —C₁-C₆ alkyl, —C₁-C₆ haloalkyl, —NH₂, —NH(C₁-C₆ alkyl), —NH(C₁-C₆ alkyl), —N(C₁-C₆ alkyl)₂, —OH, —C₁-C₆ alkoxy, —C₁-C₆ haloalkoxy, —CONH₂, —CONH(C₁-C₆ alkyl), —CON(C₁-C₆ alkyl), —CON(C₁-C₆ alkyl)₂, —CONH—OH, —CON(C₁-C₆ alkyl)-OH, —CONH₂, —OCO(C₁-C₆ alkyl), —O—CH(C₁-C₆ alkyl)₂, —CONH—NH₂, —CO₂H, or —CO₂(C₁-C₆ alkyl);
R₁₆ is hydrogen or C₁-C₆ alkyl;
each R₁₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₁ₛ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₁₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₁₉ groups when attached to the same carbon atom form ═O; and
each R₂₁ is independently of Formula (IIa):

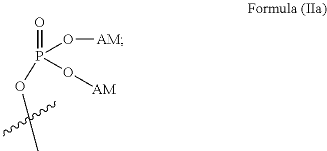

wherein each AM is independently selected from Na, Li, K, Rb, or a C₁-C₆ alkyl.

In some embodiments, X is Cl.

The present disclosure provides method of synthesis of a compound of Formula (VI). In one aspect, the method comprises the steps of
(a) contacting a compound of Formula (VI′)

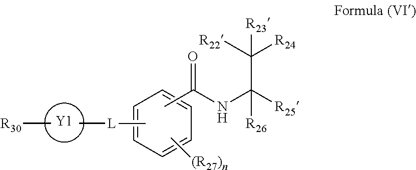

with a phosphate salt of the formula

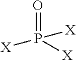

in the presence of a base catalyst to form a reaction mixture; and
(b) contacting the reaction mixture with an alkali metal hydroxide to form a compound of Formula (VI):

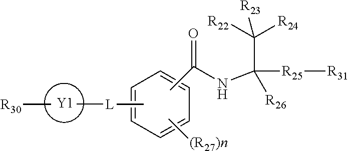

wherein
X is a halogen;
L represents a linker, wherein L is absent or is selected from the group consisting of —C≡C—C≡C—, or —C≡C—R₃₂, wherein R₃₂ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₂₉, (C₃-C₈ cycloalkyl) C₁-C₆ alkyl- optionally substituted with R₂₉, a C₃-C₇ cyclyl and heterocyclyl optionally substituted with R₂₉;
Y1 represents aryl optionally substituted with R₂₈, heteroaryl optionally substituted with R₂₈, cycloalkyl or heterocyclyl optionally substituted with R₂₈;
n is an integer selected from 0, 1, 2, 3, or 4;
R₃₀ is hydrogen, C₁-C₆ alkyl optionally substituted with R₂₉, aryl optionally substituted with R₂₉, heteroaryl optionally substituted with R₂₉, or heterocyclyl optionally substituted with R₂₉;
R₂₂ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₂′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₃ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₃ is halogen, C₁-C₆ alkyl, C₁-C₆ haloalkyl, —NH₂, —NH(C₁-C₆ alkyl), —N(C₁-C₆ alkyl)₂, —OH, —C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₄ is C₁-C₆ haloalkyl;
R₂₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₂₅′ is —C₁-C₆ alkyl, —C₁-C₆ haloalkyl, —NH₂, —NH(C₁-C₆ alkyl), —NH(C₁-C₆ alkyl), —N(C₁-C₆ alkyl)₂, —OH, —C₁-C₆ alkoxy, —C₁-C₆ haloalkoxy, —CONH₂, —CONH(C₁-C₆ alkyl), —CON(C₁-C₆ alkyl)₂, —CONH—OH, —CONH(C₁-C₆ alkyl)-OH, —CONH₂, —OCO(C₁-C₆ alkyl), —O—CH(C₁-C₆ alkyl)₂, —CONH—NH₂, —CO₂H, or —CO₂(C₁-C₆ alkyl);
R₂₆ is hydrogen or C₁-C₆ alkyl;
each R₂₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₂₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl). —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
each R₃₁ is independently a compound of Formula (IIa), Formula (IIb), or Formula (IIc):

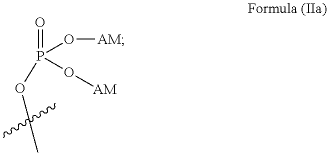

wherein each AM is independently selected from Na, Li, K, Rb, or a C₁-C₆ alkyl.

In some embodiments, X is Cl.

In yet another aspect, the synthesis method further comprises a cyclic tertiary amine as a base catalyst. In one aspect, the cyclic tertiary amine comprises a morpholine, a piperazine, a piperidine, a hydroxypiperidine, a halopiperidine, a pyrrolidine, or a N-alkyltetrahydroquinolone.

In yet another aspect, the cyclic tertiary amine is N-Methylmorpholine (NMM). In yet another aspect, step (a) is conducted at a temperature from about 0° C. to about −20° C. Yet in another aspect, the alkali metal hydroxide comprises sodium hydroxide. In yet another aspect, the compounds of the present disclosure are synthesized in an industrial scale.

Another aspect of the present disclosure provides all that is described and illustrated herein.

### BRIEF DESCRIPTION OF THE DRAWINGS

The accompanying Figures and Examples are provided by way of illustration and not by way of limitation. The foregoing aspects and other features of the disclosure are explained in the following description, taken in connection with the accompanying example figures (also “FIG.”) relating to one or more embodiments.

FIG. 1A and FIG. 1B provide exemplary phosphate prodrugs of the present invention.

FIG. 2A-FIG. 2D provide characterization of synthesized compound LPC-233-Phos-Pro.

FIG. 2A provides a ¹H NMR spectrum of LPC-233-Phos-Pro. FIG. 2B provides a ¹³C NMR spectrum of LPC-233-Phos-Pro. FIG. 2C provides a mass spectrum of LPC-233-Phos-Pro. FIG. 2D provides a UV Chromatogram of LPC-233-Phos-Pro.

FIG. 3 provides the solubility comparison between LPC-233 and LPC-233-Phos-Pro in Captisol.

FIG. 4A-FIG. 4C provide data showing LPC-233-Phos-Pro rescues mice with lethal Acinetobacter infection in a dose-dependent manner. FIG. 4A is a graph showing the antibiotic effects of a 100 mg/kg dose of LPC-233 against Acinetobacter infection in mice over a period of 14 days. FIG. 4B is a graph showing the antibiotic effects of a 100 mg/kg dose of LPC-233-Phos-Pro against Acinetobacter infection in mice over a period of 14 days. FIG. 4C is a graph showing antibiotic effects of LPC-233-Phos-Pro against Acinetobacter infection in three different doses: 100 mg/kg, 30 mg/kg, and 10 mg/kg.

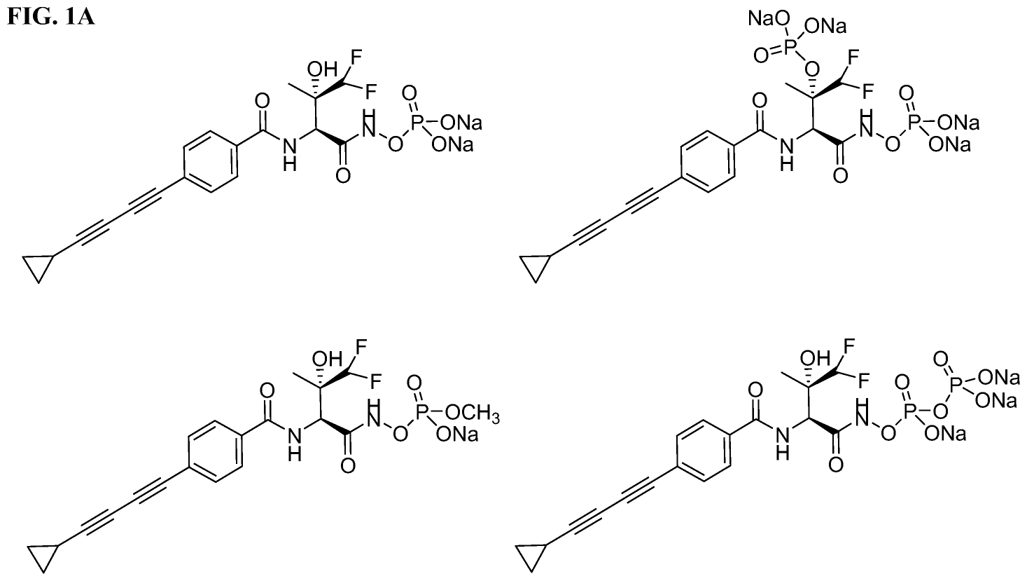

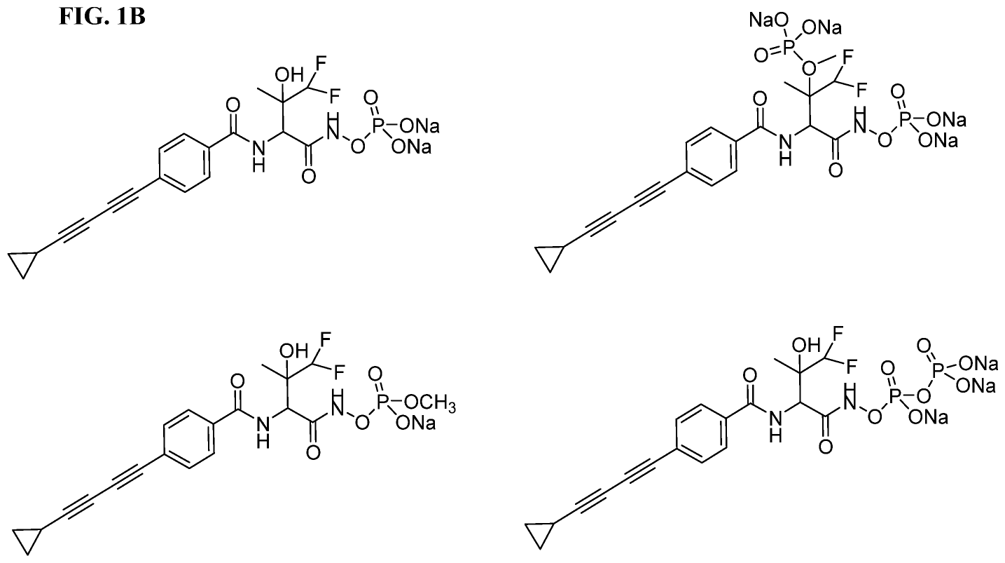

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

### A. Definitions

Articles “a” and “an” are used herein to refer to one or to more than one (i.e. at least one) of the grammatical object of the article. By way of example, “an element” means at least one element and can include more than one element.

“About” is used to provide flexibility to a numerical range endpoint by providing that a given value may be “slightly above” or “slightly below” the endpoint without affecting the desired result.

The use herein of the terms “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition. As used herein, the terms “prevent,” “preventing,” “prevention.” “prophylactic treatment” and the like refer to reducing the probability of developing a disease, disorder or condition in a subject, who does not have, but is at risk of or susceptible to developing a disease, disorder or condition. The term “effective amount” or “therapeutically effective amount” refers to an amount sufficient to effect beneficial or desirable biological and/or clinical results.

As used herein, the term “administering” an agent, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to a subject by any suitable route for delivery of the therapeutic agent to the desired location in the animal, including delivery by either the parenteral or oral route, intramuscular injection, subcutaneous/intradermal injection, intravenous injection, intrathecal administration, buccal administration, transdermal delivery, topical administration, and administration by the intranasal or respiratory tract route.

The term “biological sample” as used herein includes, but is not limited to, a sample containing tissues, cells, and/or biological fluids isolated from a subject. Examples of biological samples include, but are not limited to, tissues, cells, biopsies, blood, lymph, serum, plasma, urine, saliva, mucus and tears. A biological sample may be obtained directly from a subject (e.g., by blood or tissue sampling) or from a third party (e.g., received from an intermediary, such as a healthcare provider or lab technician).

The term “disease” as used herein includes, but is not limited to, any abnormal condition and/or disorder of a structure or a function that affects a part of an organism. It may be caused by an external factor, such as an infectious disease, or by internal dysfunctions, such as cancer, cancer metastasis, and the like.

In some embodiments, the disease comprises a cancer. As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma, and the like.

In other embodiments, the disease comprises an infectious disease. In some embodiments, the infectious disease comprises a bacterial infection. In certain embodiments, the bacterial infection comprises a Gram-negative bacterial infection. In one embodiment, the Gram-negative bacteria is selected from the group consisting of Pseudomonas aeruginosa, Stenotrophomonas maltophilia, Burkholderia cepacia, Alcaligenes xylosoxidans, Acinetobacter, Enterobacteriaceae, Haemophilus, Neisseria, Francisella tularensis, Yersinia pestis, Burkholderia pseudomallei, Burkholderia mallei, Rickettsia prowazekii, Coxiella burnetti, Campylobacter jejuni, Shigella, Moraxella catarrhalis, Chlamydia trachomatis, and combinations thereof. In another embodiment, the Gram-negative bacteria comprises Neisseria gonorrhoeae. Yet in another embodiment, the Gram-negative bacteria comprises Acinetobacter Baumannii. Yet in another embodiment, the Gram-negative bacteria comprises an Enterobacteriacacae. Yet in another embodiment, the Enterobacteriaceae is selected from the group consisting of Serratia, Proteus, Klebsiella, Enterobacter, Citrobacter, Salmonella, Providencia, Morganella, Cedecea, Edwardsiella, Escherichia coli, Enterobacter cloacae, Enterobacter aerogenes, and combinations thereof.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient).

Terms used herein may be preceded and/or followed by a single dash, or a double dash, “=”, to indicate the bond order of the bond between the named substituent and its parent moiety; a single dash indicates a single bond and a double dash indicates a double bond. In the absence of a single or double dash it is understood that a single bond is formed between the substituent and its parent moiety; further, substituents are intended to be read “left to right” unless a dash indicates otherwise. For example, C₁-C₆ alkoxycarbonyloxy and —OC(O)C₁-C₆ alkyl indicate the same functionality; similarly, arylalkyl and -alkylaryl indicate the same functionality.

The term “alkenyl” as used herein, means a straight or branched chain hydrocarbon containing from 2 to 10 carbons, unless otherwise specified, and containing at least one carbon-carbon double bond. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, 3-decenyl, and 3,7-dimethylocta-2,6-dienyl.

The term “alkoxy” as used herein, means an alkyl group, as defined herein, appended to the parent molecular moiety through an oxygen atom. Representative examples of alkoxy include, but are not limited to, methoxy, ethoxy, propoxy, 2-propoxy, butoxy, tert-butoxy, pentyloxy, and hexyloxy.

The term “alkyl” as used herein, means a straight or branched chain hydrocarbon containing from 1 to 10 carbon atoms unless otherwise specified. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl. When an “alkyl” group is a linking group between two other moieties, then it may also be a straight or branched chain; examples include, but are not limited to —CH₂—, —CH₂CH₂—, —CH₂CH₂CHC(CH₃)—, and —CH₂CH(CH₂CH₃)CH₂—.

The term “alkylene” refers to a bivalent alkyl group. An “alkylene chain” is a polymethylene group, i.e., —(CH₂)n-, wherein n is a positive integer, preferably from one to six, from one to four, from one to three, from one to two, or from two to three. A substituted alkylene chain is a polymethylene group in which one or more methylene hydrogen atoms is replaced with a substituent. Suitable substituents include those described below for a substituted aliphatic group. An alkylene chain also may be substituted at one or more positions with an aliphatic group or a substituted aliphatic group.

The term “alkynyl” as used herein, means a straight or branched chain hydrocarbon group containing from 2 to 10 carbon atoms and containing at least one carbon-carbon triple bond. Representative examples of alkynyl include, but are not limited, to acetylenyl, 1-propynyl, 2-propynyl, 3-butynyl, 2-pentynyl, and 1-butynyl.

The term “aryl,” as used herein, means a phenyl (i.e., monocyclic aryl), or a bicyclic ring system containing at least one phenyl ring or an aromatic bicyclic ring containing only carbon atoms in the aromatic bicyclic ring system. The bicyclic aryl can be azulenyl, naphthyl, or a phenyl fused to a monocyclic cycloalkyl, a monocyclic cycloalkenyl, or a monocyclic heterocyclyl. The bicyclic aryl is attached to the parent molecular moiety through any carbon atom contained within the phenyl portion of the bicyclic system, or any carbon atom with the napthyl or azulenyl ring. The fused monocyclic cycloalkyl or monocyclic heterocyclyl portions of the bicyclic aryl are optionally substituted with one or two oxo and/or thia groups. Representative examples of the mono- and bicyclic aryls include, but are not limited to, phenyl, pyridinyl, pyrimidinyl, pyrrolyl, furanyl, aziridinyl, pyrrolidinyl, pyrrolidonyl, thiophenyl, piperidinyl, thiazol, azulenyl, naphthyl, dihydroinden-1-yl, dihydroinden-2-yl, dihydroinden-3-yl, dihazulenyl, naphthyl, dihydroinden-1-yl, dihydroinden-2-yl, dihydroinden-3-yl, dihydroinden-4-yl, 2,3-dihydroindol-4-yl, 2,3-dihydroindol-5-yl, 2,3-dihydroindol-6-yl, 2,3-dihydroindol-7-yl, inden-1-yl, inden-2-yl, inden-3-yl, inden-4-yl, dihydronaphthalen-2-yl, dihydronaphthalen-3-yl, dihydronaphthalen-4-yl, dihydronaphthalen-1-yl, 5,6,7,8-tetrahydronaphthalen-1-yl, 5,6,7,8-tetrahydronaphthalen-2-yl, 2,3-dihydrobenzofuran-4-yl, 2,3-dihydrobenzofuran-5-yl, 2,3-dihydrobenzofuran-6-yl, 2,3-dihydrobenzofuran-7-yl, benzo[d][1,3]dioxol-4-yl, benzo[d][1,3]dioxol-5-yl, 2H-chromen-2-one-5-yl, 2H-chromen-2-one-6-yl, 2H-chromen-2-one-7-yl, 2H-chromen-2-one-8-yl, isoindoline-1,3-dion-4-yl, isoindoline-1,3-dion-5-yl, inden-1-on-4-yl, inden-1-on-5-yl, inden-1-on-6-yl, inden-1-on-7-yl, 2,3-dihydrobenzo[b][1,4]dioxan-5-yl, 2,3-dihydrobenzo[b][1.4]dioxan-6-yl, 2H-benzo[b][1,4]oxazin-3(4H)-one-5-yl, 2H-benzo[b][1,4]oxazin-3(4H)-one-6-yl, 2H-benzo[b][1,4]oxazin-3(4H)-one-7-yl, 2H-benzo[b][1,4]oxazin-3(4H)-one-8-yl, benzo[d]oxazin-2(3H)-one-5-yl, benzo[d]oxazin-2(3H)-one-6-yl, benzo[d]oxazin-2(3H)-one-7-yl, benzo[d]oxazin-2(3H)-one-8-yl, quinazolin-4(3H)-one-5-yl, quinazolin-4(3H)-one-6-yl, quinazolin-4(3H)-one-7-yl, quinazolin-4(3H)-one-8-yl, quinoxalin-2(1H)-one-5-yl, quinoxalin-2(1H)-one-6-yl, quinoxalin-2(1H)-one-7-yl, quinoxalin-2(1H)-one-8-yl, benzo[d]thiazol-2(3H)-one-4-yl, benzo[d]thiazol-2(3H)-one-5-yl, benzo[d]thiazol-2(3H)-one-6-yl, and benzo[d]thiazol-2(3H)-one-7-yl. In certain embodiments, the bicyclic aryl is (i) naphthyl or (ii) a phenyl ring fused to either a 5 or 6 membered monocyclic cycloalkyl, a 5 or 6 membered monocyclic cycloalkenyl, or a 5 or 6 membered monocyclic heterocyclyl, wherein the fused cycloalkyl, cycloalkenyl, and heterocyclyl groups are optionally substituted with one or two groups which are independently oxo or thia.

The terms “cyano” and “nitrile” as used herein, mean a —CN group.

The term “cycloalkyl” as used herein, means a monocyclic or a bicyclic cycloalkyl ring system. In one aspect, the cycloalkyl may have one or more hetero atom(s). Monocyclic ring systems are cyclic hydrocarbon groups containing from 3 to 8 carbon atoms, where such groups can be saturated or unsaturated, but not aromatic. In certain embodiments, cycloalkyl groups are fully saturated. Examples of monocyclic cycloalkyls include cyclopropyl, cyclobutyl, cyclopentyl, cyclopentenyl, cyclohexyl, cyclohexenyl, cycloheptyl, cyclooctyl, ethyleniminyl, or tetrahydrofuranyl. Bicyclic cycloalkyl ring systems are bridged monocyclic rings or fused bicyclic rings. Bridged monocyclic rings contain a monocyclic cycloalkyl ring where two non-adjacent carbon atoms of the monocyclic ring are linked by an alkylene bridge of between one and three additional carbon atoms (i.e., a bridging group of the form —(CH₂)_{w}—, where w is 1, 2, or 3). Representative examples of bicyclic ring systems include, but are not limited to, bicyclo[3,1,1]heptane, bicyclo[2.2.1]heptane, bicyclo[2,2,2]octane, bicyclo[3,2,2]nonane, bicyclo[3,3,1]nonane, and bicyclo[4,2,1]nonane. Fused bicyclic cycloalkyl ring systems contain a monocyclic cycloalkyl ring fused to either a phenyl, a monocyclic cycloalkyl, a monocyclic cycloalkenyl, a monocyclic heterocyclyl, or a monocyclic heteroaryl. The bridged or fused bicyclic cycloalkyl is attached to the parent molecular moiety through any carbon atom contained within the monocyclic cycloalkyl ring. Cycloalkyl groups are optionally substituted with one or two groups which are independently oxo or thia. In certain embodiments, the fused bicyclic cycloalkyl is a 5 or 6 membered monocyclic cycloalkyl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused bicyclic cycloalkyl is optionally substituted by one or two groups which are independently oxo or thia.

The term “halo” or “halogen” as used herein, means —Cl, —Br, —I or —F.

The terms “haloalkyl” and “haloalkoxy” refer to an alkyl or alkoxy group, as the case may be, which is substituted with one or more halogen atoms.

The term “heteroaryl,” as used herein, means a monocyclic heteroaryl or a bicyclic ring system containing at least one heteroaromatic ring. The monocyclic heteroaryl can be a 5 or 6 membered ring. The 5 membered ring consists of two double bonds and one, two, three or four nitrogen atoms and optionally one oxygen or sulfur atom. The 6 membered ring consists of three double bonds and one, two, three or four nitrogen atoms. The 5 or 6 membered heteroaryl is connected to the parent molecular moiety through any carbon atom or any nitrogen atom contained within the heteroaryl. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, a monocyclic cycloalkyl, a monocyclic cycloalkenyl, a monocyclic heterocyclyl, or a monocyclic heteroaryl. The fused cycloalkyl or heterocyclyl portion of the bicyclic heteroaryl group is optionally substituted with one or two groups which are independently oxo or thia. When the bicyclic heteroaryl contains a fused cycloalkyl, cycloalkenyl, or heterocyclyl ring, then the bicyclic heteroaryl group is connected to the parent molecular moiety through any carbon or nitrogen atom contained within the monocyclic heteroaryl portion of the bicyclic ring system. When the bicyclic heteroaryl is a monocyclic heteroaryl fused to a benzo ring, then the bicyclic heteroaryl group is connected to the parent molecular moiety through any carbon atom or nitrogen atom within the bicyclic ring system. Representative examples of bicyclic heteroaryl include, but are not limited to, benzimidazolyl, benzofuranyl, benzothienyl, benzoxadiazolyl, benzoxathiadiazolyl, benzothiazolyl, cinnolinyl, 5,6-dihydroquinolin-2-yl, 5,6-dihydroisoquinolin-1-yl, furopyridinyl, indazolyl, indolyl, isoquinolinyl, naphthyridinyl, quinolinyl, purinyl, 5,6,7,8-tetrahydroquinolin-2-yl, 5,6,7,8-tetrahydroquinolin-3-yl, 5,6,7,8-tetrahydroquinolin-4-yl, 5,6,7,8-tetrahydroisoquinolin-1-yl, thienopyridinyl, 4,5,6,7-tetrahydrobenzo[c][1,2,5]oxadiazolyl, and 6,7-dihydrobenzo[c][1,2,5]oxadiazol-4(5H)-onyl. In certain embodiments, the fused bicyclic heteroaryl is a 5 or 6 membered monocyclic heteroaryl ring fused to either a phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the fused cycloalkyl, cycloalkenyl, and heterocyclyl groups are optionally substituted with one or two groups which are independently oxo or thia.

The terms “heterocyclyl” and “heterocycloalkyl” as used herein, mean a monocyclic heterocycle or a bicyclic heterocycle. The monocyclic heterocycle is a 3, 4, 5, 6 or 7 membered ring containing at least one heteroatom independently selected from the group consisting of O, N, and S where the ring is saturated or unsaturated, but not aromatic. The 3 or 4 membered ring contains 1 heteroatom selected from the group consisting of O, N and S. The 5 membered ring can contain zero or one double bond and one, two or three heteroatoms selected from the group consisting of O, N and S. The 6 or 7 membered ring contains zero, one or two double bonds and one, two or three heteroatoms selected from the group consisting of O, N and S. The monocyclic heterocycle is connected to the parent molecular moiety through any carbon atom, or any nitrogen atom contained within the monocyclic heterocycle. Representative examples of monocyclic heterocycle include, but are not limited to, azetidinyl, azepanyl, aziridinyl, diazepanyl, 1,3-dioxanyl, 1,3-dioxolanyl, 1,3-dithiolanyl, 1,3-dithianyl, imidazolinyl, imidazolidinyl, isothiazolinyl, isothiazolidinyl, isoxazolinyl, isoxazolidinyl, morpholinyl, oxadiazolinyl, oxadiazolidinyl, oxazolinyl, oxazolidinyl, piperazinyl, piperidinyl, pyranyl, pyrazolinyl, pyrazolidinyl, pyrrolinyl, pyrrolidinyl, tetrahydrofuranyl, tetrahydrothienyl, thiadiazolinyl, thiadiazolidinyl, thiazolinyl, thiazolidinyl, thiomorpholinyl, 1,1-dioxidothiomorpholinyl (thiomorpholine sulfone), thiopyranyl, and trithianyl. The bicyclic heterocycle is a monocyclic heterocycle fused to either a phenyl, a monocyclic cycloalkyl, a monocyclic cycloalkenyl, a monocyclic heterocycle, or a monocyclic heteroaryl. The bicyclic heterocycle is connected to the parent molecular moiety through any carbon atom or any nitrogen atom contained within the monocyclic heterocycle portion of the bicyclic ring system. Representative examples of bicyclic heterocyclyls include, but are not limited to, 2,3-dihydrobenzofuran-2-yl, 2,3-dihydrobenzofuran-3-yl, indolin-1-yl, indolin-2-yl, indolin-3-yl, 2,3-dihydrobenzothien-2-yl, decahydroquinolinyl, decahydroisoquinolinyl, octahydro-1H-indolyl, and octahydrobenzofuranyl. Heterocyclyl groups are optionally substituted with one or two groups which are independently oxo or thia. In certain embodiments, the bicyclic heterocyclyl is a 5 or 6 membered monocyclic heterocyclyl ring fused to phenyl ring, a 5 or 6 membered monocyclic cycloalkyl, a 5 or 6 membered monocyclic cycloalkenyl, a 5 or 6 membered monocyclic heterocyclyl, or a 5 or 6 membered monocyclic heteroaryl, wherein the bicyclic heterocyclyl is optionally substituted by one or two groups which are independently oxo or thia.

The term “oxo” as used herein means a ═O group.

The term “saturated” as used herein means the referenced chemical structure does not contain any multiple carbon-carbon bonds. For example, a saturated cycloalkyl group as defined herein includes cyclohexyl, cyclopropyl, and the like.

The term “substituted”, as used herein, means that a hydrogen radical of the designated moiety is replaced with the radical of a specified substituent, provided that the substitution results in a stable or chemically feasible compound. The term “substitutable”, when used in reference to a designated atom, means that attached to the atom is a hydrogen radical, which can be replaced with the radical of a suitable substituent.

The phrase “one or more” substituents, as used herein, refers to a number of substituents that equals from one to the maximum number of substituents possible based on the number of available bonding sites, provided that the above conditions of stability and chemical feasibility are met. Unless otherwise indicated, an optionally substituted group may have a substituent at each substitutable position of the group, and the substituents may be either the same or different. As used herein, the term “independently selected” means that the same or different values may be selected for multiple instances of a given variable in a single compound.

The term “thia” as used herein means a ═S group.

The term “unsaturated” as used herein means the referenced chemical structure contains at least one multiple carbon-carbon bond, but is not aromatic. For example, a unsaturated cycloalkyl group as defined herein includes cyclohexenyl, cyclopentenyl, cyclohexadienyl, and the like.

Unless otherwise defined, all technical terms used herein have the same meaning as commonly understood by one of ordinary skill in the art to which this disclosure belongs.

### B. Compositions

The present disclosure is based, in part, on the discovery by the inventors of novel formulation approaches for hydroximate-based compounds to enhance solubility, efficacy, safety, and the like. In particular, such approaches utilize a phosphate synthesis scheme to generate a hydroxamate-based-phosphate prodrug which exhibits enhanced solubility, efficacy and safety. The methods provided herein can be used on any hydroxamate-based compound.

Thus, in a broad aspect, the present disclosure comprises a hydroxamate-based phosphate prodrug comprising, consisting of, or consisting essentially of a hydroxamate-based compound a phosphate group.

In another aspect, the disclosure encompasses the compounds of formulae (I), (IV), and (VI) shown below, pharmaceutical compositions containing those compounds, and methods of using such compounds to treat and/or prevent bacterial infections.

Thus, one aspect of the disclosure provides compounds of Formula (I):

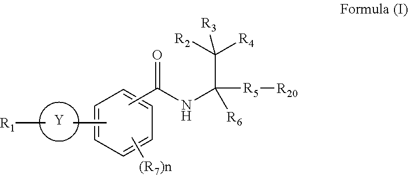

or a stereo-isomer, an enantiomer, a polymorph, a pharmaceutically acceptable salt, an ester, or a derivative thereof. In Formula (I), Y represents aryl optionally substituted with R₈, heteroaryl optionally substituted with R₈, cycloalkyl, or heterocyclyl optionally substituted with R₈;
n is an integer selected from 0, 1, 2, 3, or 4;
R₁ is hydrogen, C₁-C₆ alkyl optionally substituted with R₉, aryl optionally substituted with R₉, heteroaryl optionally substituted with R₉, or heterocyclyl optionally substituted with R₉;
R₂ is halogen, C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₃ is halogen, C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₄ is C₁-C₆ haloalkyl;
R₅ is —C₁-C₆ alkyl-, —C₁-C₆ haloalkyl-, —NH—, —NH(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₆ is hydrogen or C₁-C₆ alkyl;
each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl). —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups that when attached to the same carbon atom form ═O;
each R₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups that when attached to the same carbon atom form ═O; and
each R₂₀ is independently of Formula (IIa), Formula (IIb), or Formula (IIc):

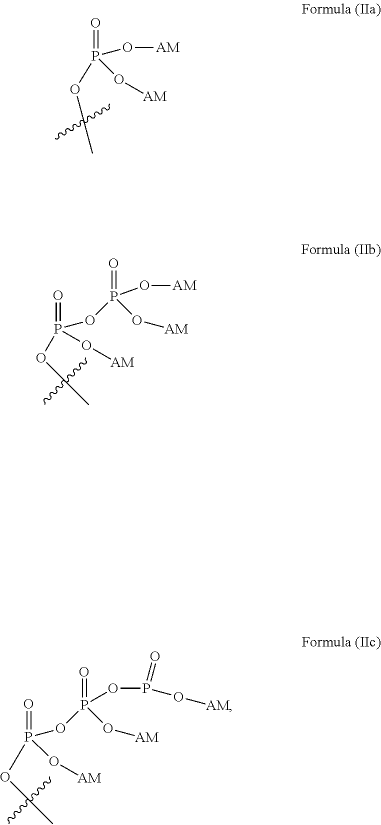

wherein each AM is independently selected from Na, Li, K, Rb, or a C₁-C₆ alkyl. In an exemplary embodiment, each AM is independently Na or methyl.

In some embodiments, R₂ is selected from C₁-C₆ alkyl, hydroxyl, or —O—R₂₀; R₃ is selected from C₁-C₆ alkyl, hydroxyl, or —O—R₂₀; and R₅ is selected from the group consisting of —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, and —CONH—NH—.

In an exemplary embodiment, R₂ is methyl, R₃ is hydroxyl, R₄ is difluoromethyl, and R₅ is —CONH—. In another exemplary embodiment, R₂ is methyl, R₃ is —O—R₂₀. R₄ is difluoromethyl, and R₅ is —CONH—.

In some embodiments, the moiety of Formula (I)

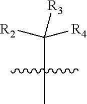

has a structure of Formula (IIIa) or Formula (IIIb):

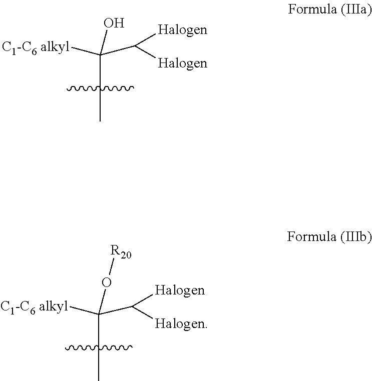

In some additional embodiments, Y is selected from the group of consisting of phenyl, pyridinyl, pyrimidinyl, ethyleniminyl, cyclopropyl, pyrrolyl, furanyl, tetrahydrofuranyl, aziridinyl, pyrrolidinyl, pyrrolidonyl, thiophenyl, piperidinyl, thiazol, azulenyl, naphthyl, and imidazole, which are optionally substituted with R₈.

Further provided herein are compounds of Formula (IV):

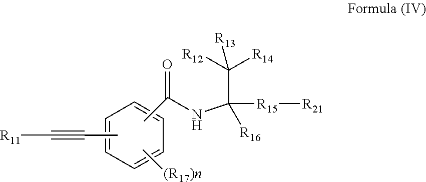

or a stereo-isomer, an enantiomer, a polymorph, a pharmaceutically acceptable salt, an ester or a derivative thereof, wherein
n is an integer selected from 0, 1, 2, 3, or 4;
R₁₁ is —C≡C—R₃₃, C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈;
wherein R₃₃ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₉, (C₃-C₈ cycloalkyl) C₁-C₆ alkyl-optionally substituted with R₁₉, a C₃-C₇ cyclyl or heterocyclyl optionally substituted with R₁₉;
R₁₂ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₃ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₄ is C₁-C₆ haloalkyl;
R₁₅ is —C₁-C₆ alkyl-, —C₁-C₆ haloalkyl-, —NH—, —NH(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₁₆ is hydrogen or C₁-C₆ alkyl;
each R₁₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₁₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —C₁-C₆ alkyl-OH, —C₁-C₆ alkyl-(C₁-C₆ alkoxy), —C₁-C₆ alkyl-NH₂, —C₁-C₆alkyl-NH—C₁-C₆ alkyl), —C₁-C₆ alkyl-N(C₁-C₆ alkyl)₂, —C₁-C₆ alkyl-NH(SO₂ C₁-C₆ alkyl), —CONH₂, —CONH(C₁-C₆ alkyl), —CON(C₁-C₆ alkyl)₂, —NH(SO₂—C₁-C₆ alkyl), —CONH—OH, —CONH—OCO(C₁-C₆ alkyl), —C(NH)NH—OH, —CONH—NH₂, —CO(C₁-C₆ alkyl), —CO₂H. —CO₂(C₁-C₆ alkyl), —C₁-C₆ alkyl-CONH₂, —C₁-C₆ alkyl-CONH(C₁-C₆ alkyl), —C₁-C₆ alkyl-CON(C₁-C₆ alkyl)₂, —C₁-C₆ alkyl-CONH—OH, —C₁-C₆ alkyl-CO(C₁-C₆ alkyl), —C₁-C₆ alkyl-CO₂H, and —C₁-C₆ alkyl-CO₂(C₁-C₆ alkyl); or two R₁₈ groups that when attached to the same carbon atom form ═O;
each R₁₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —C₁-C₆ alkyl-OH, —C₁-C₆ alkyl-(C₁-C₆ alkoxy), —C₁-C₆ alkyl-NH₂, —C₁-C₆ alkyl-NH—C₁-C₆ alkyl), —C₁-C₆ alkyl-N(C₁-C₆ alkyl)₂, —C₁-C₆ alkyl-NH(SO₂—C₁-C₆ alkyl), —CONH₂, —CON(C₁-C₆ alkyl), —CON(C₁-C₆ alkyl)₂, —NH(SO₂ C₁-C₆ alkyl), —CONH—OH, —CONH—OCO(C₁-C₆ alkyl), —C(NH)NH—OH, —CONH—NH₂, —CO(C₁-C₆ alkyl), —CO₂H, —CO₂(C₁-C₆ alkyl), —C₁-C₆ alkyl-CONH₂, —C₁-C₆ alkyl-CON(C₁-C₆ alkyl), —C₁-C₆ alkyl- CON(C₁-C₆ alkyl)₂, —C₁-C₆ alkyl-CONH—OH, —C₁-C₆ alkyl-CO(C₁-C₆ alkyl), —C₁-C₆ alkyl-CO₂H, and —C₁-C₆ alkyl-CO₂(C₁-C₆ alkyl); or two R₁₉ groups that when attached to the same carbon atom form ═O; and
each R₂₁ is independently selected from Formula (IIa), Formula (IIb), or Formula (IIc):

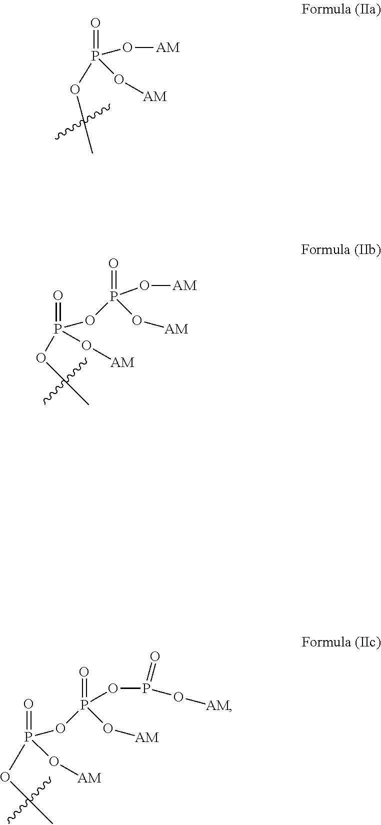

wherein each AM is independently selected from Na, Li, K, Rb, or a C₁-C₆ alkyl. In an exemplary embodiment, each AM is independently Na or methyl.

In some embodiments, R₁₂ is selected from C₁-C₆ alkyl, hydroxyl, or —O—R₂₁; R₁₃ is selected from C₁-C₆ alkyl, hydroxyl, or —O—R₂₁; and R₁₅ is selected from the group consisting of —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, and —CONH—NH—. In some embodiments, the moiety

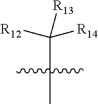

is of Formula (Va) or Formula (Vb):

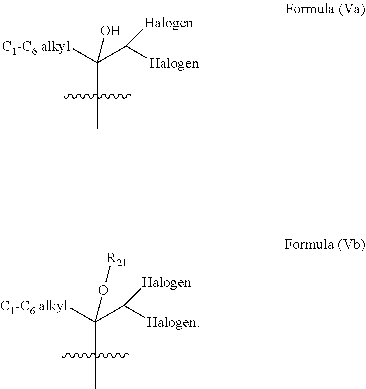

In some embodiments, R₁₁ is selected from —C≡C—R₃₃, or C₃-C₈ cycloalkyl optionally substituted with R₁₈, (C₃-C₈ cycloalkyl)-C₁-C₆ alkyl- optionally substituted with R₁₈, or heterocyclyl optionally substituted with R₁₈; and R₃₃ is —C₁-C₆ alkyl-CO₂H, C₃-C₈ cycloalkyl optionally substituted with R₁₉, (C₃-C₈ cycloalkyl)-C₁-C₆ alkyl-, optionally substituted with R₁₉, a C₃-C₇ cyclyl or heterocyclyl optionally substituted with R₁₉.

In an exemplary embodiment, R₁₂ is methyl, R₁₃ is hydroxyl, R₁₄ is difluoromethyl, and R₁₅ is —CONH—. In another exemplary embodiment, R₁₂ is methyl, R₁₃ is —O—R₂₁, R₁₄ is difluoromethyl, and R₁₅ is —CONH—.

Further provided herein are compounds of Formula (VI):

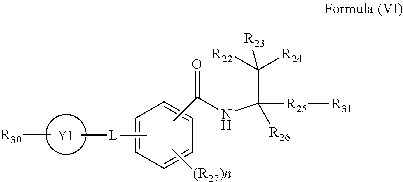

or a stereo-isomer, an enantiomer, a polymorph, a pharmaceutically acceptable salt, an ester, or a derivative thereof, wherein
L represents a linker, wherein L is absent or is selected from the group consisting of —C≡C—C≡C—, or —C≡C—R₃₂—, wherein R₃₂ is C₁-C₆ alkyl, —C₁-C₆ haloalkyl-, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₂₉, (C₃-C₈ cycloalkyl)-C₁-C₆ alkyl- optionally substituted with R₂₉, a C₃-C₇ cyclyl, and heterocyclyl optionally substituted with R₂₉;
Y1 represents aryl optionally substituted with R₂₈, heteroaryl optionally substituted with R₂₈, cycloalkyl, or heterocyclyl optionally substituted with R₂₈;
n is an integer selected from 0, 1, 2, 3, or 4;
R₃₀ is hydrogen, C₁-C₆ alkyl optionally substituted with R₂₉, aryl optionally substituted with R₂₉, heteroaryl optionally substituted with R₂₉, or heterocyclyl optionally substituted with R₂₉;
R₂₂ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₃ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₄ is C₁-C₆ haloalkyl;
R₂₅ is —C₁-C₆ alkyl-, —C₁-C₆ haloalkyl-, —NH—, —NH(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₂₆ is hydrogen or C₁-C₆ alkyl;
each R₂₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₂₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups that when attached to the same carbon atom form ═O;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups that when attached to the same carbon atom form ═O; and
each R₃₁ is independently a compound of Formula (IIa), Formula (IIb), or Formula (IIc):

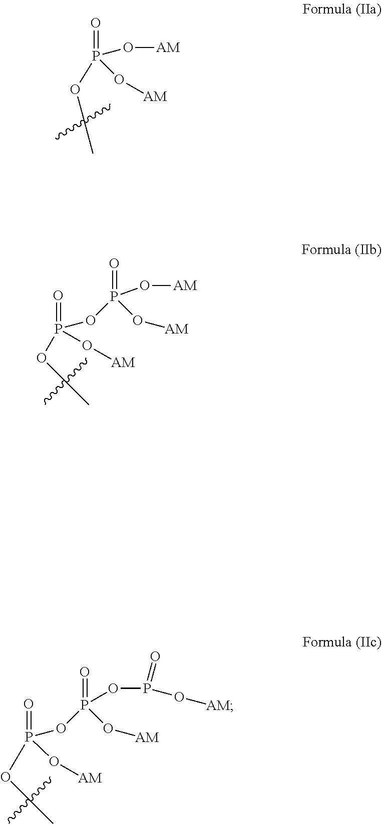

and each AM is independently selected from Na, Li, K, Rb, or a C₁-C₆ alkyl. In an exemplary embodiment, each AM is independently Na or methyl.

In one aspect of compounds of the Formula (VI), R₂₂ is selected from C₁-C₆ alkyl, hydroxyl, or —O—R₃₁; R₂₃ is selected from C₁-C₆ alkyl, hydroxyl, or —O—R₃₁; and R₂₅ is selected from the group consisting of —CONH—, —CONH(C₁-C₆ alkyl)-, —CON(C₁-C₆ alkyl)₂-, —CONH—O—, —CONH(C₁-C₆ alkyl)-O—, —CONH—, —OCO(C₁-C₆ alkyl)-, and —CONH—NH—.

In another aspect of compounds of the Formula (VI), its moiety

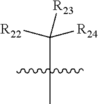

is of Formula (VIIa) or Formula (VIIb):

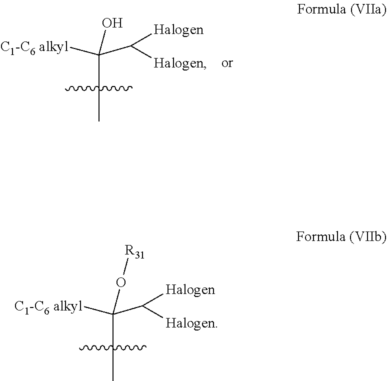

In yet another aspect of compounds of Formula (VI), Y1 is selected from the group consisting of phenyl, pyridinyl, pyrimidinyl, ethyleniminyl, cyclopropyl, pyrrolyl, furanyl, tetrahydrofuranyl, aziridinyl, pyrrolidinyl, pyrrolidonyl, thiophenyl, piperidinyl, thiazol, azulenyl, naphthyl, and imidazole, which are optionally substituted with R₂₈.

In an exemplary embodiment, R₂₂ is methyl, R₂₃ is hydroxyl, R₂₄ is difluoromethyl, and R₂₅ is —CONH—. In another exemplary embodiment, R₂₂ is methyl, R₂₃ is —O—R₃₁, R₂₄ is difluoromethyl, and R₂₅ is —CONH—.

In another embodiment, the present disclosure provides a compound having a formula selected from:

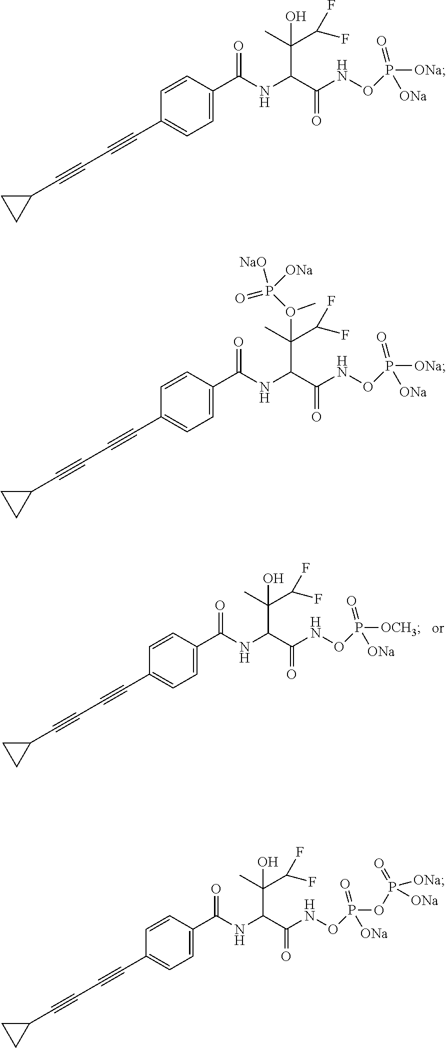

or a polymorph, a pharmaceutically acceptable salt, an ester or a derivative thereof.

In a preferred embodiment, the present disclosure provides a compound having a formula selected from:

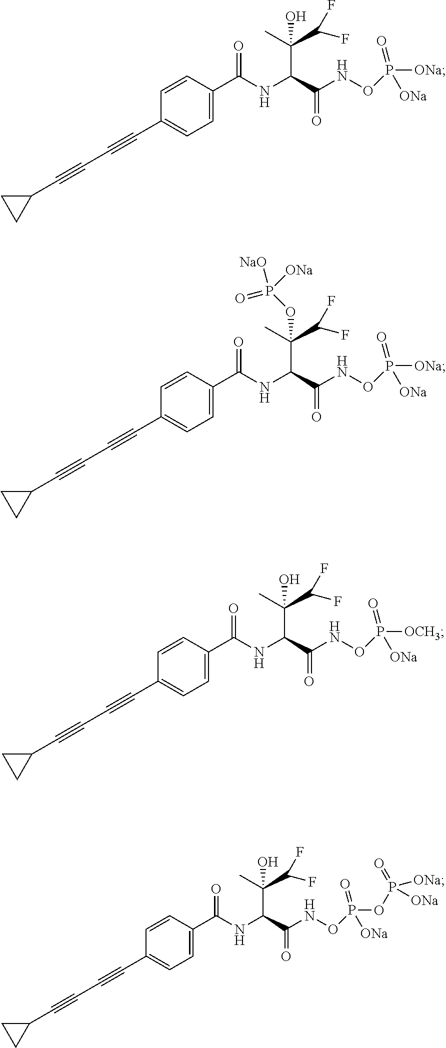

or a stereo-isomer, an enantiomer, a polymorph, a pharmaceutically acceptable salt, an ester or a derivative thereof.

In another aspect, the compound of the present disclosure comprises one of the following compounds:

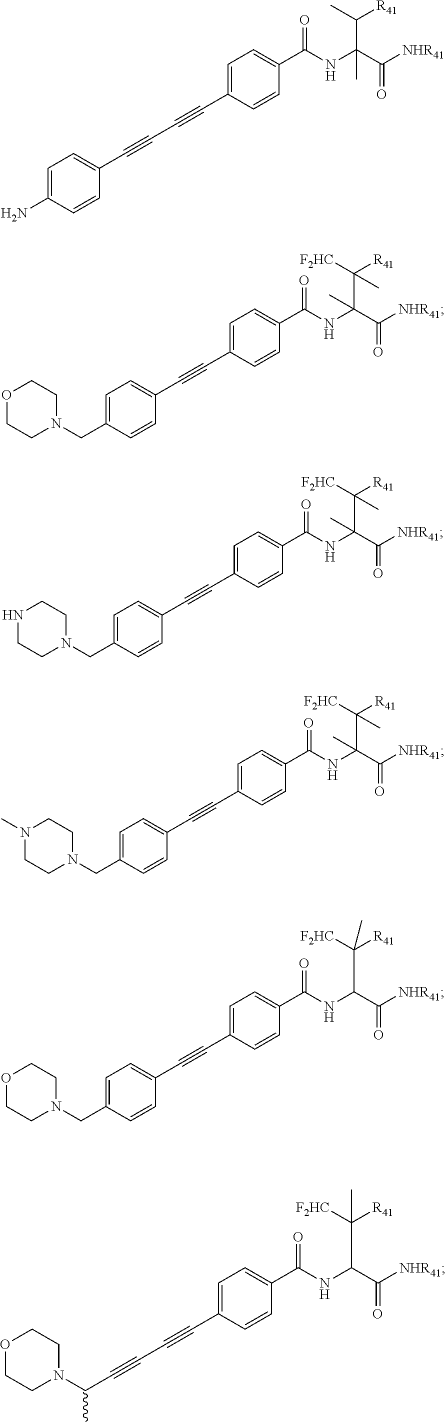

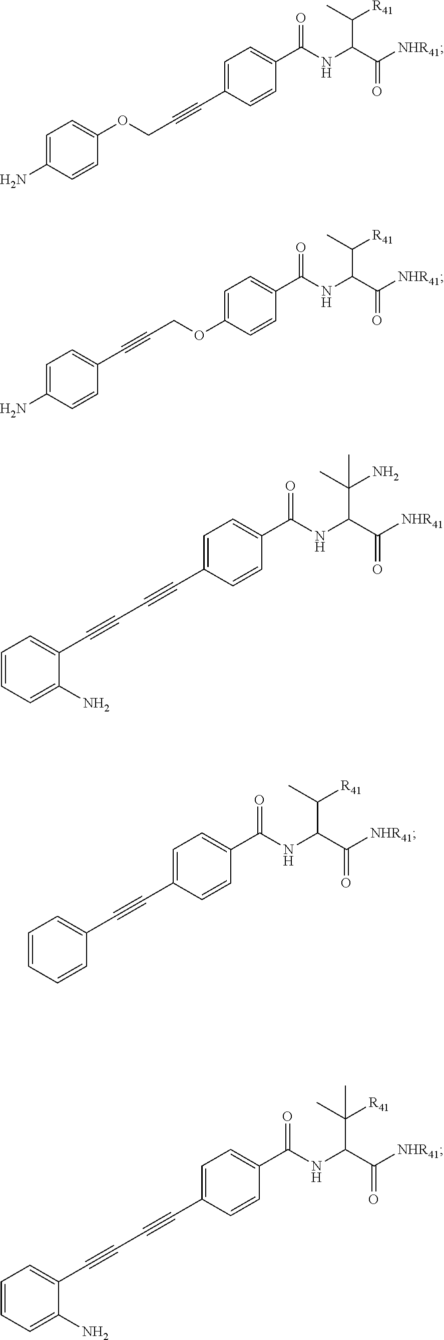

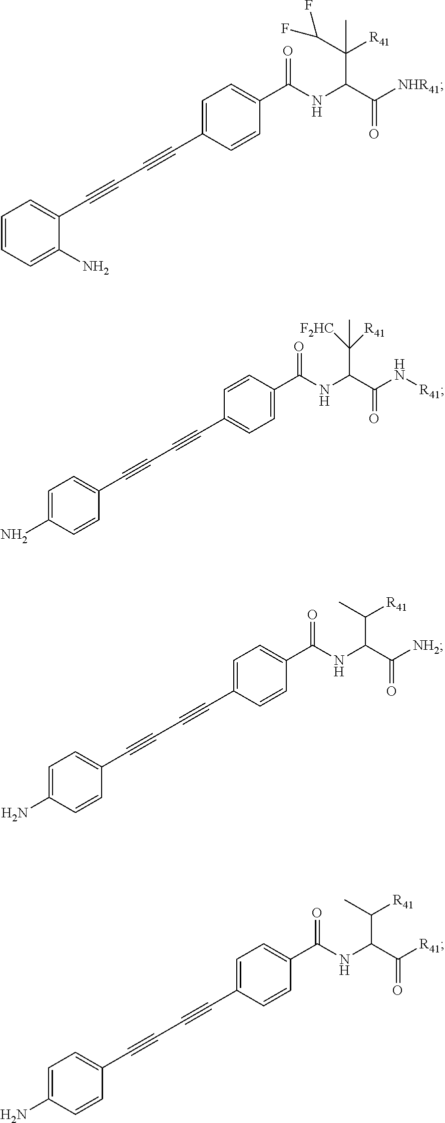

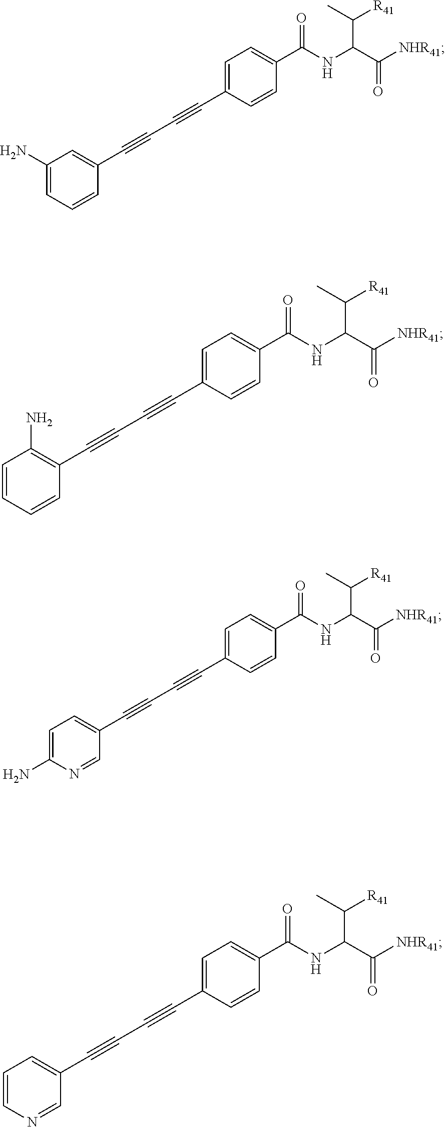

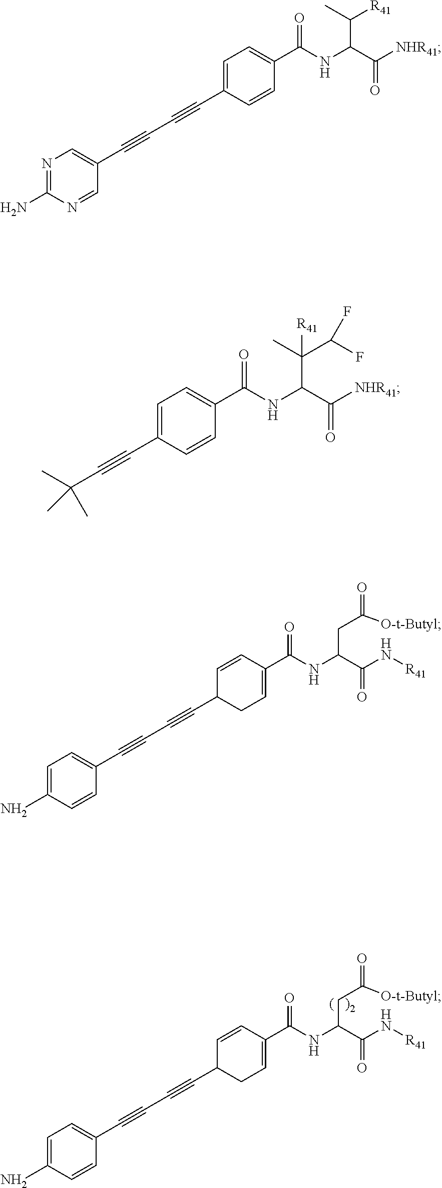

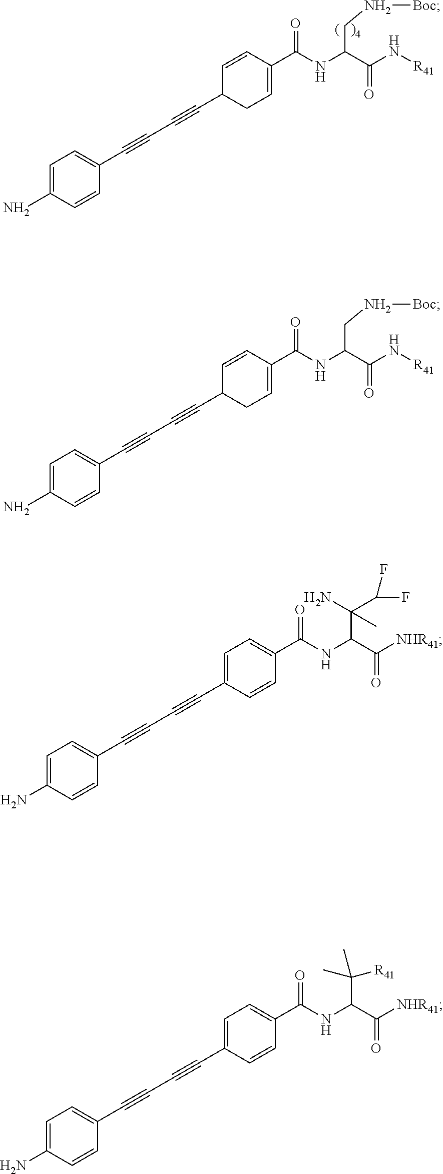

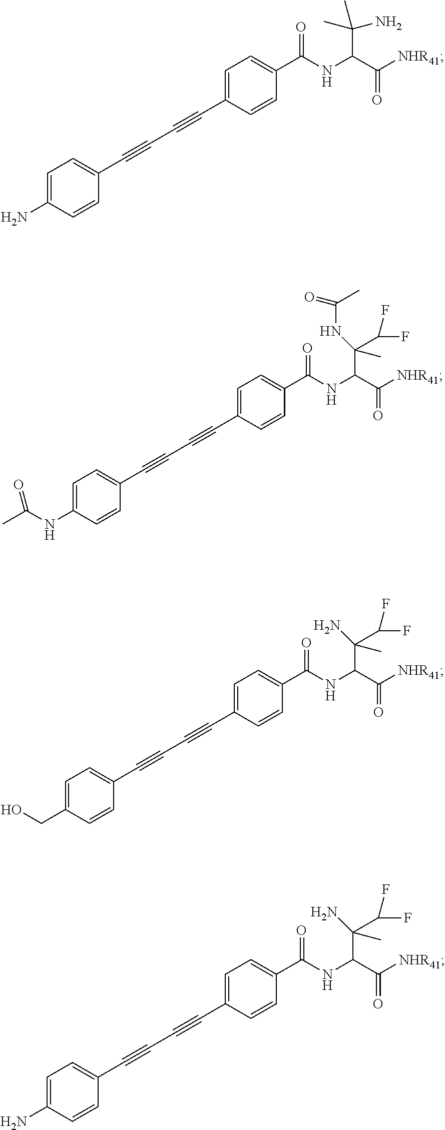

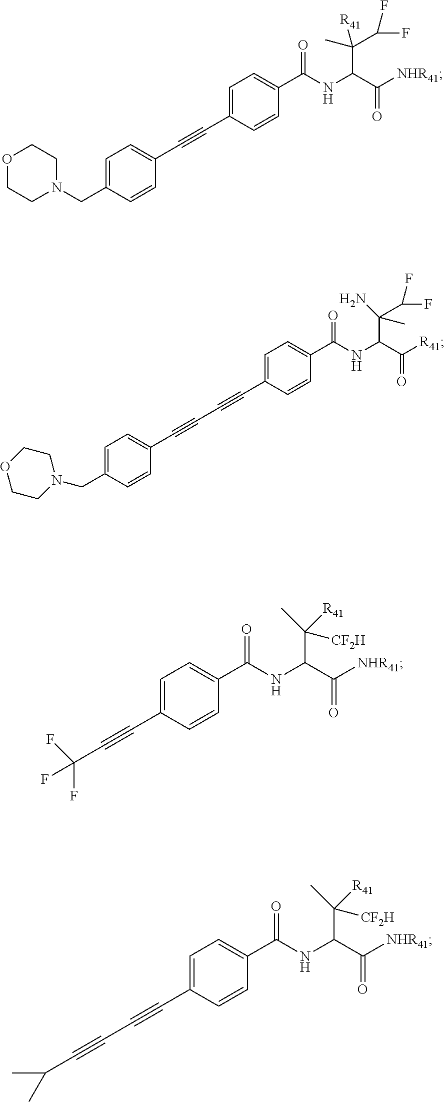

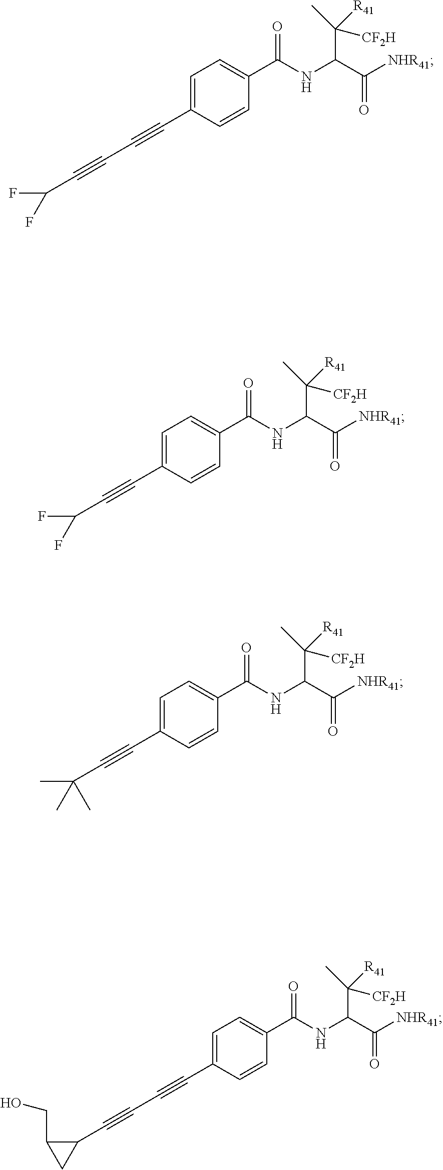

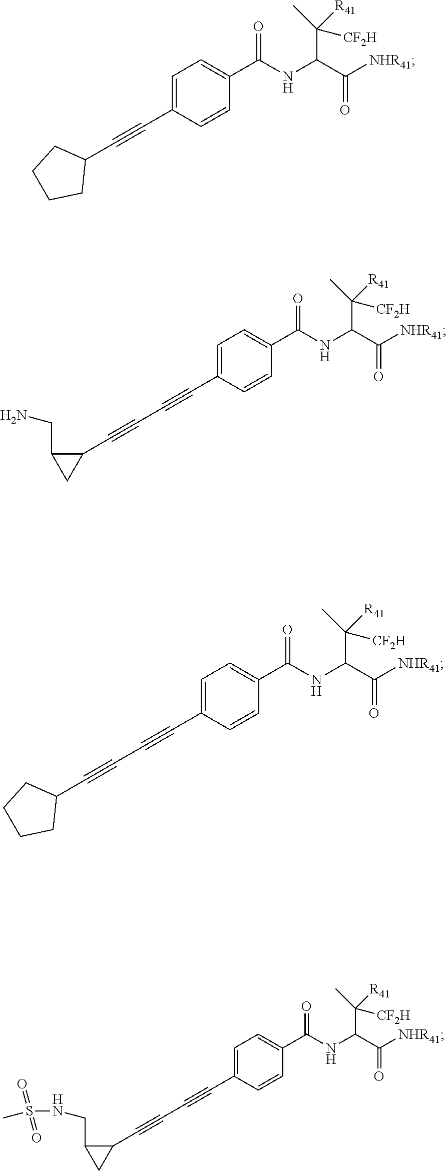

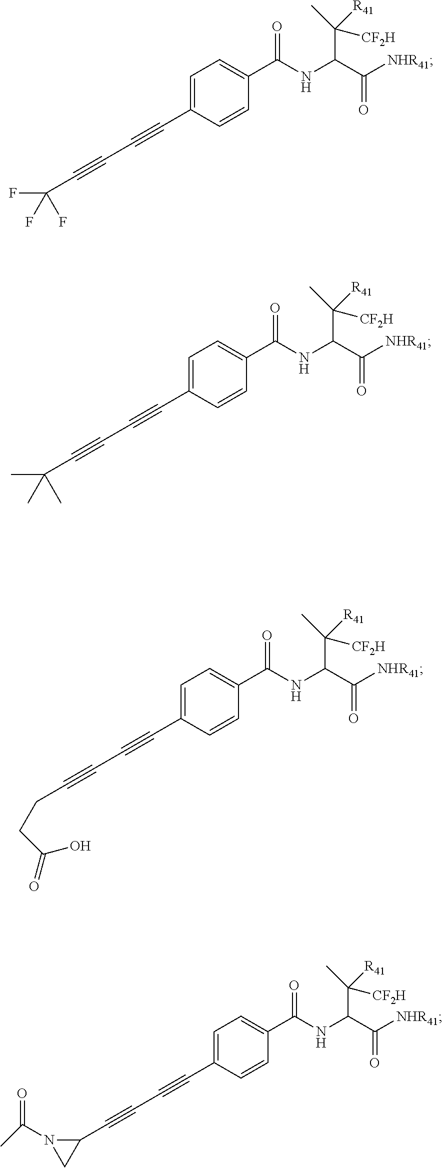

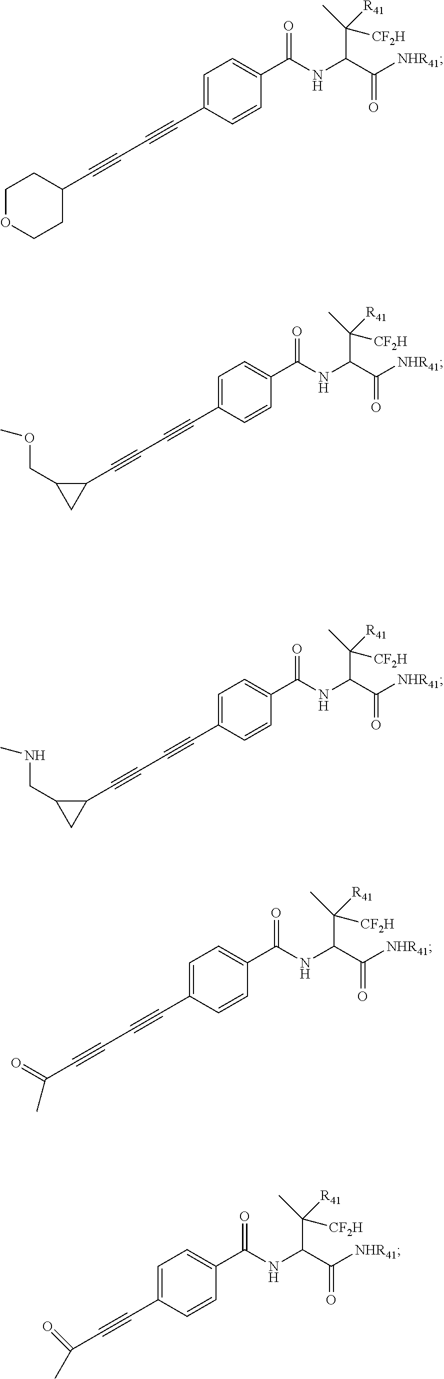

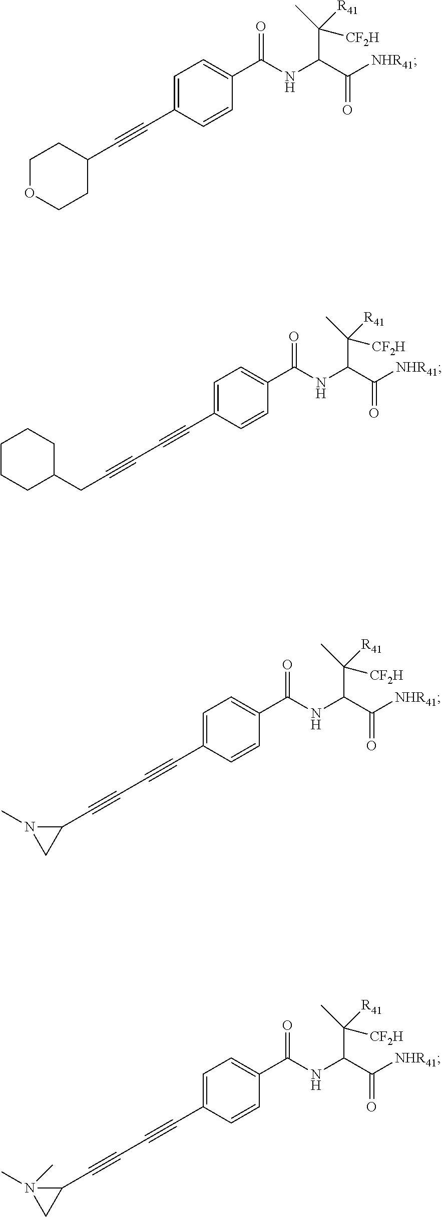

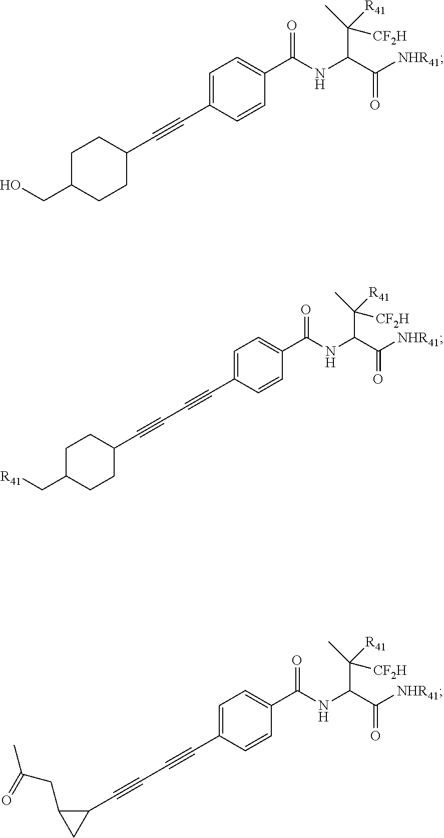

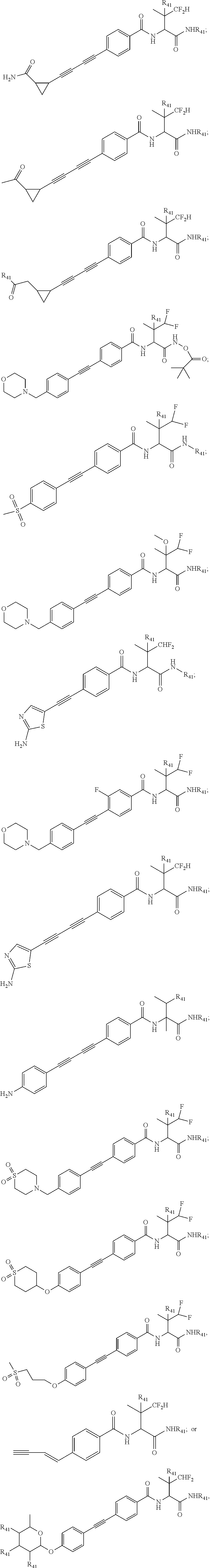

wherein each R₄₁ is independently selected from —OH, Formula (IIa), Formula (IIb), or Formula (IIc),

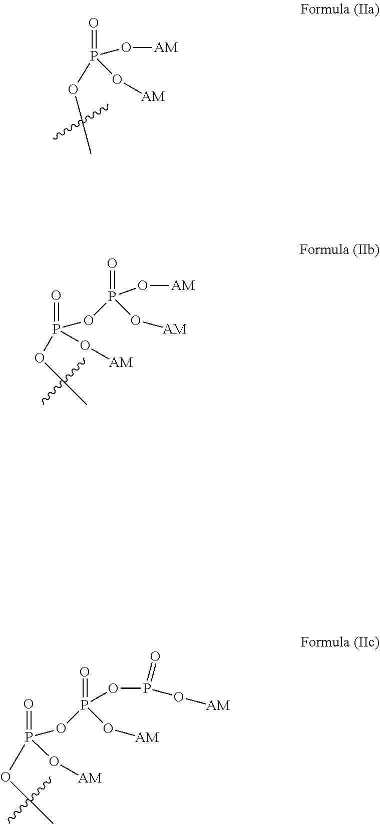

and wherein each AM is independently selected from Na, Li, K, Rb, or a C₁-C₆ alkyl. In some embodiments, at least one R₄₁ in the compound is —OH.

In another aspect, the present disclosure provides mono-phosphate, di-phosphate (i.e., pyrophosphate) or tri-phosphate prodrugs of a compound selected from the following formulas, and the phosphate is formed with a hydroxyl group or a hydroxamate oxo group of the compound:

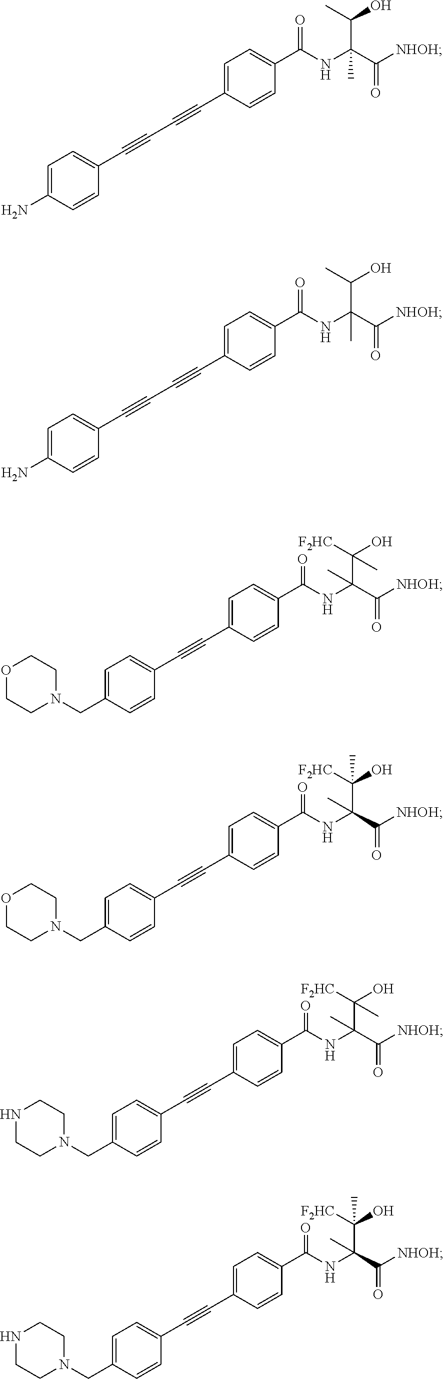

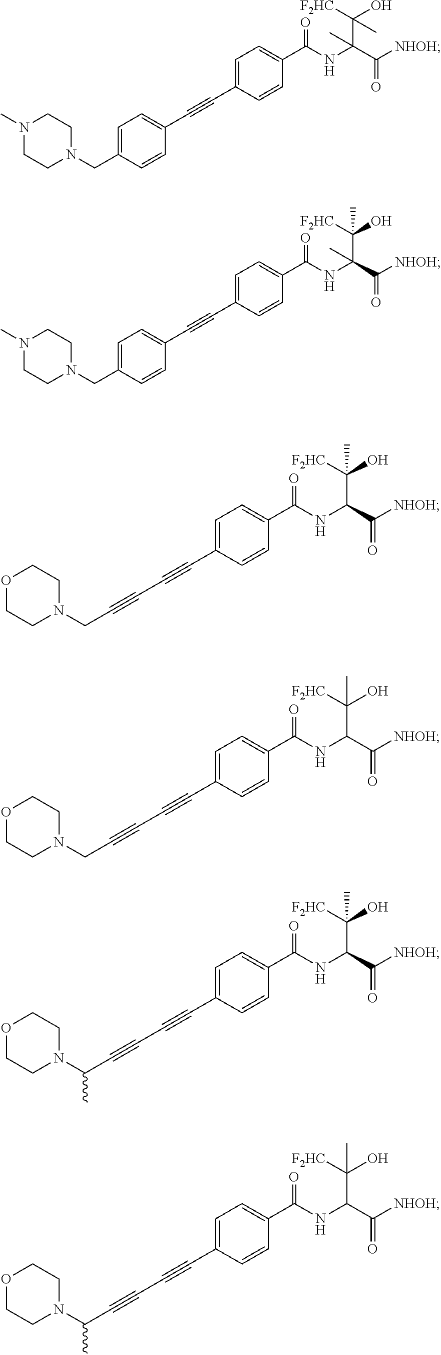

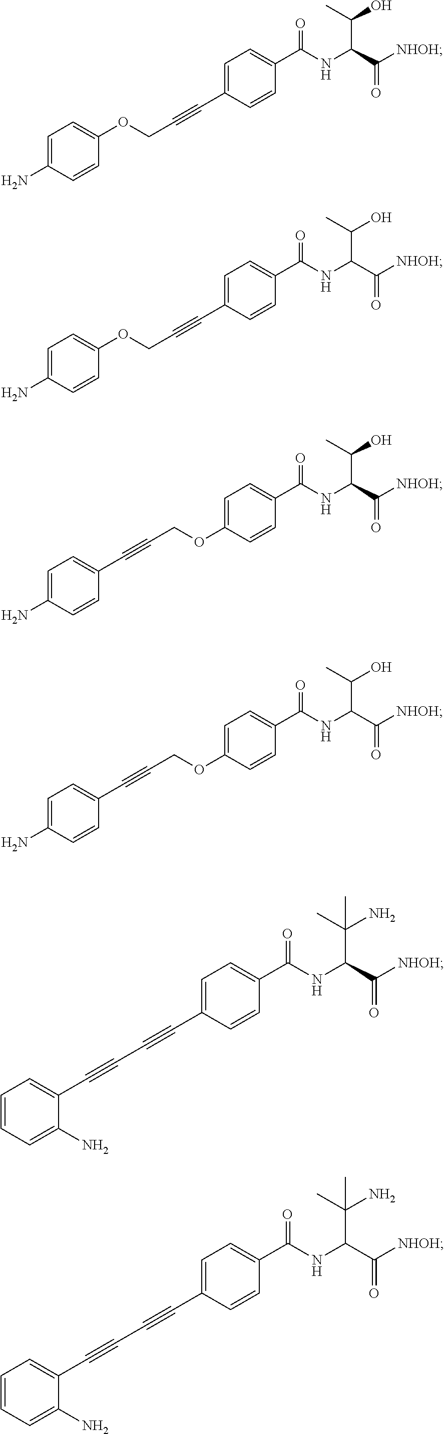

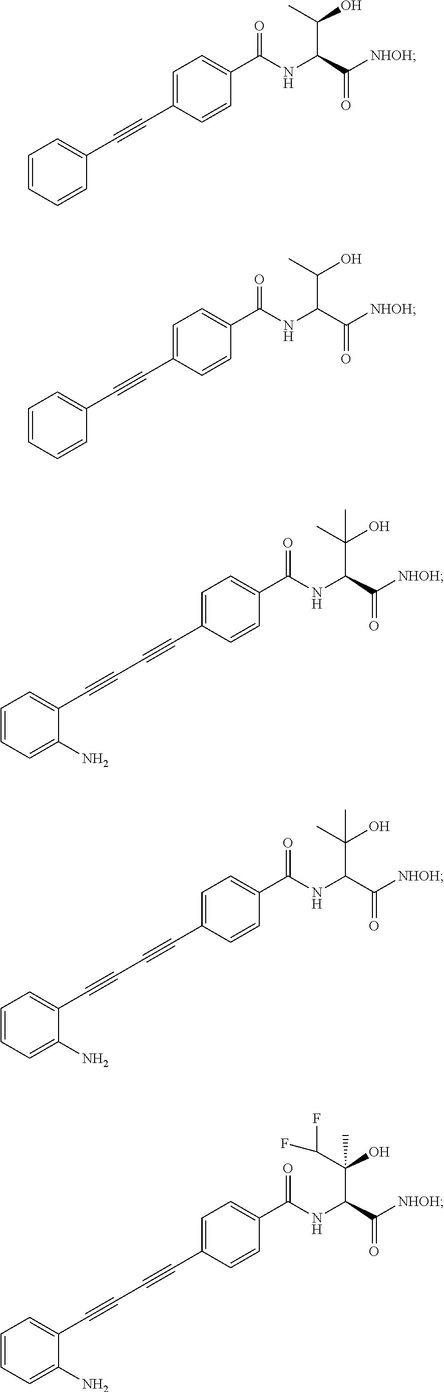

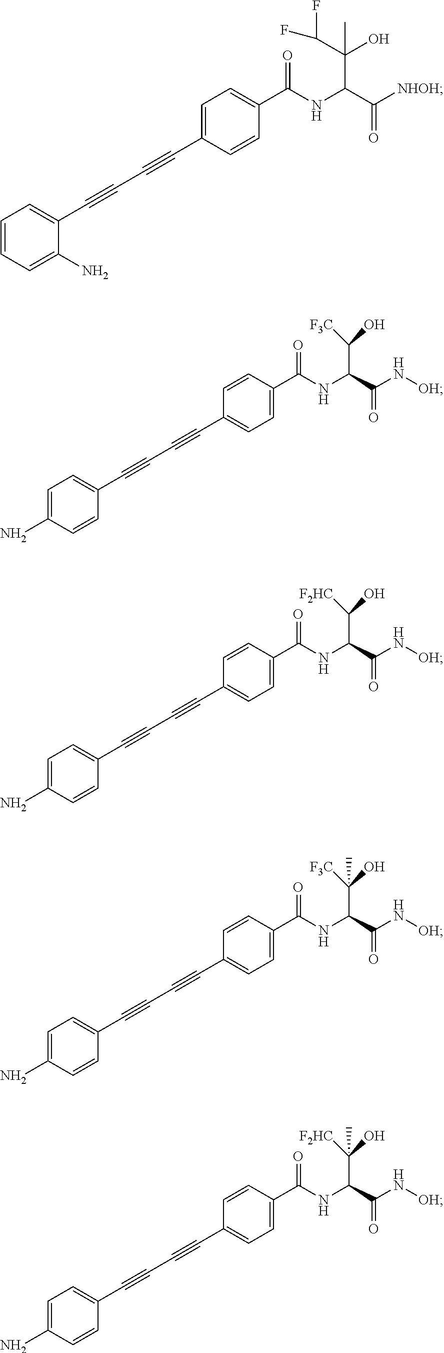

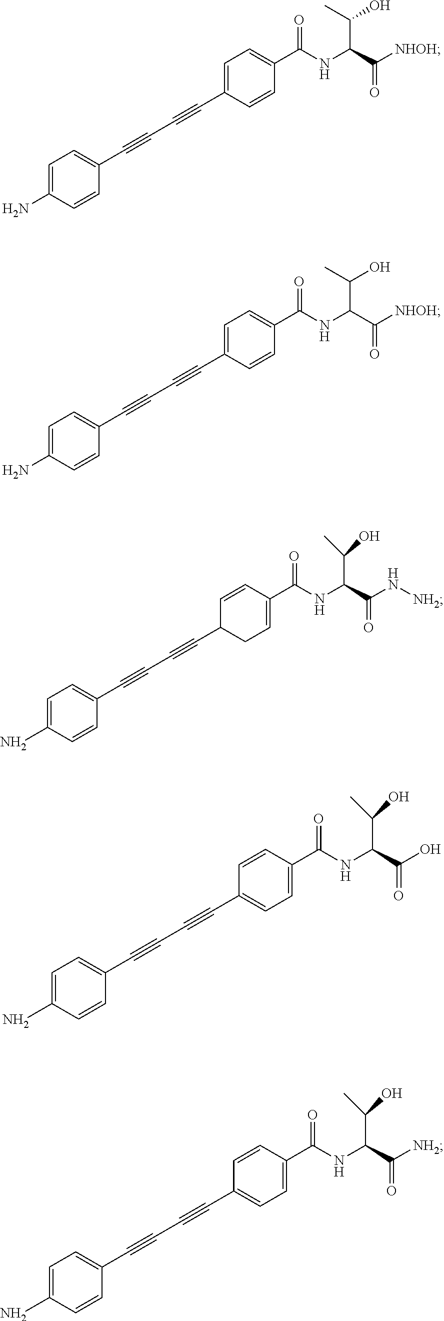

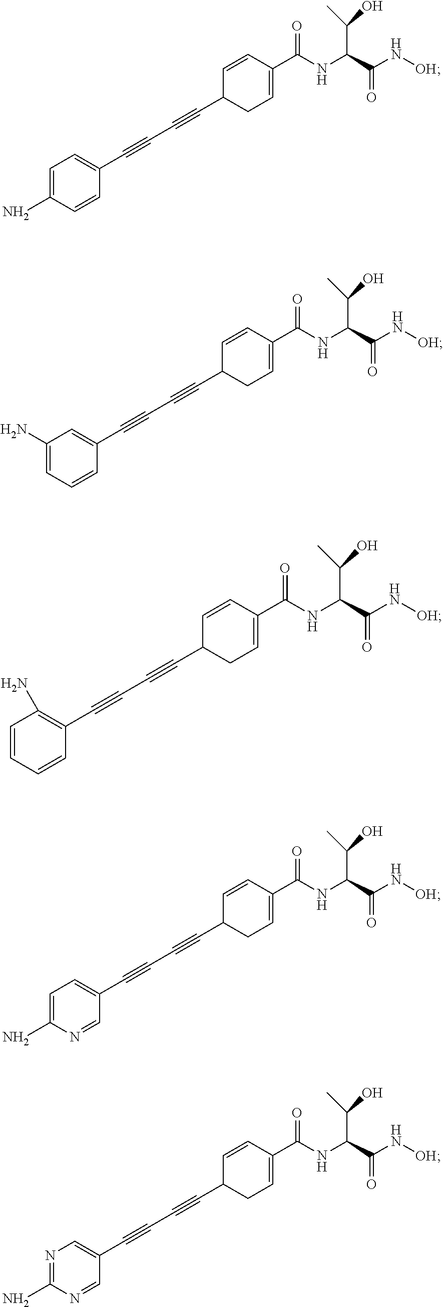 [SMILES: C[C@@H](O)[C@H](NC(=O)C1=CCC(C#CC#Cc2ccc(N)cc2)C=C1)C(=O)NO ; C[C@@H](O)[C@H](NC(=O)C1=CCC(C#CC#Cc2cccc(N)c2)C=C1)C(=O)NO ; C[C@@H](O)[C@H](NC(=O)C1=CCC(C#CC#Cc2ccccc2N)C=C1)C(=O)NO ; C[C@@H](O)[C@H](NC(=O)C1=CCC(C#CC#Cc2ccc(N)nc2)C=C1)C(=O)NO ; C[C@@H](O)[C@H](NC(=O)C1=CCC(C#CC#Cc2cnc(N)nc2)C=C1)C(=O)NO]

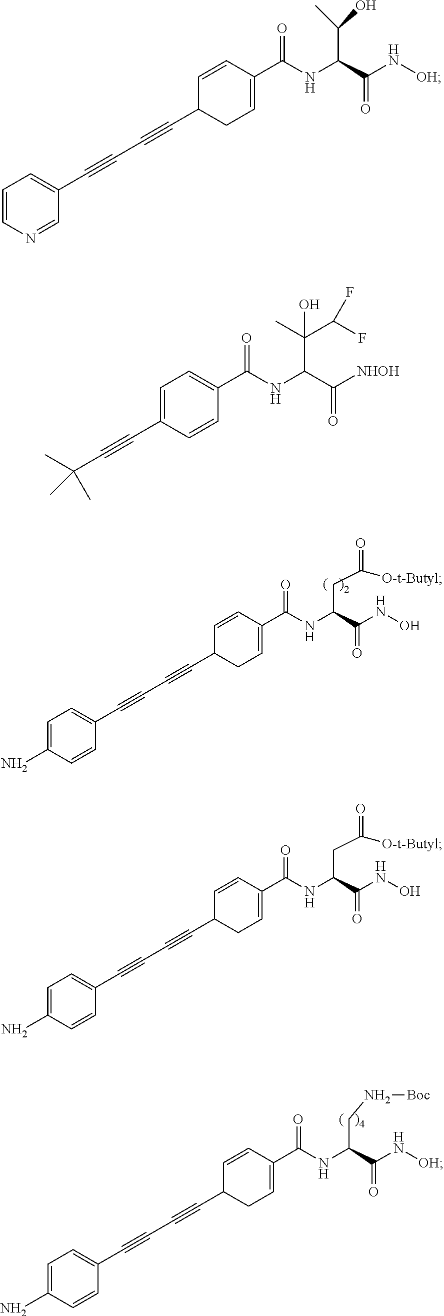

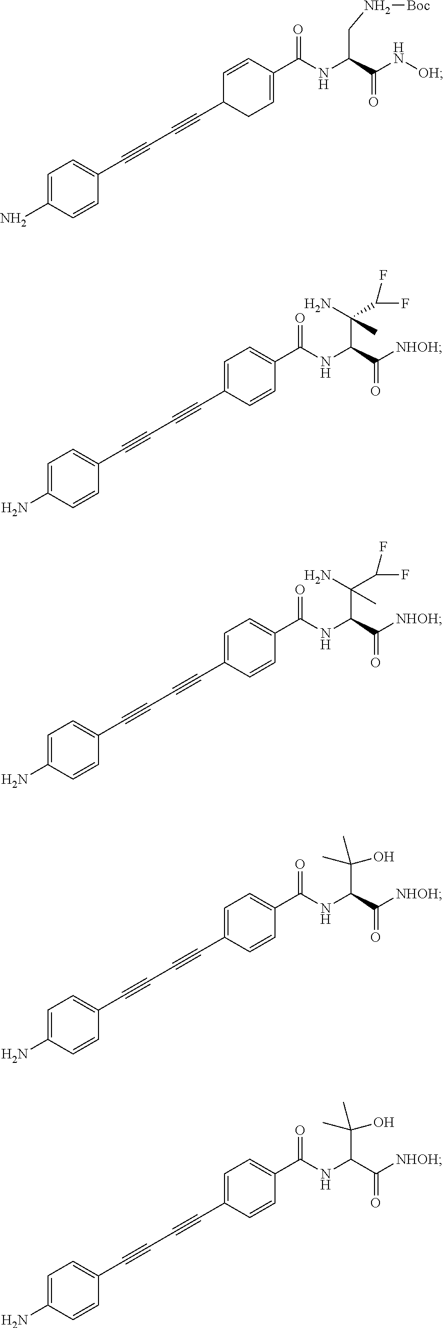

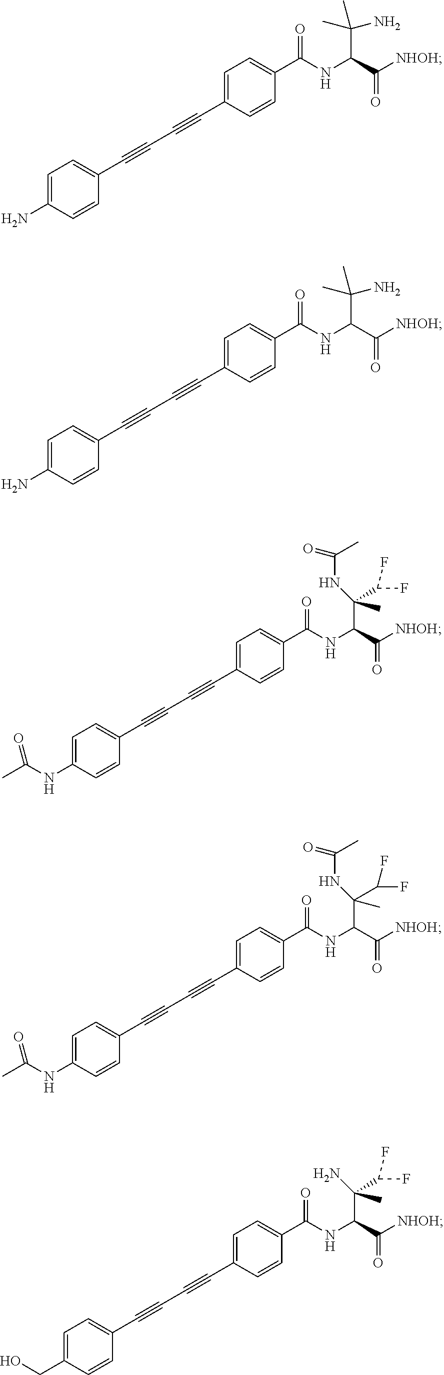

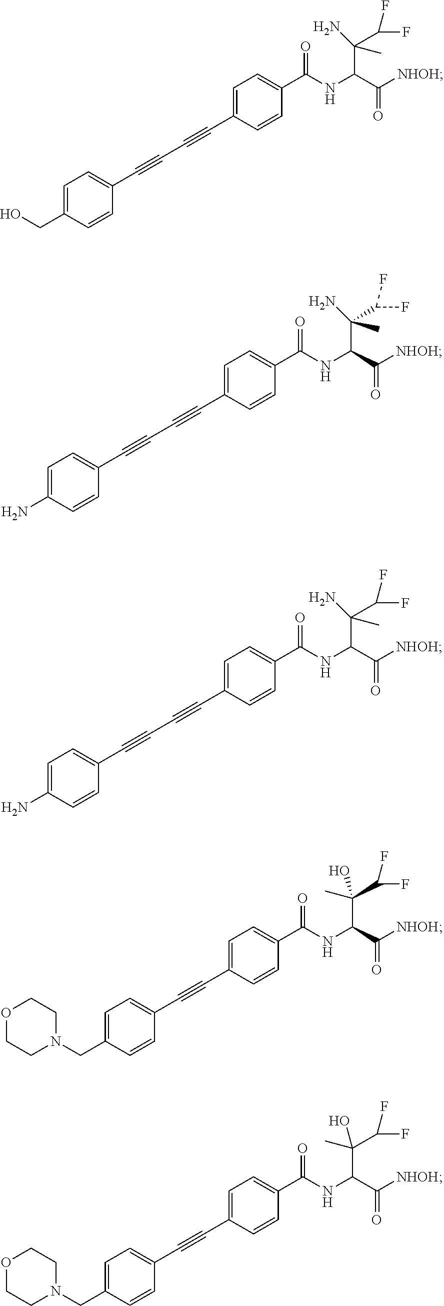

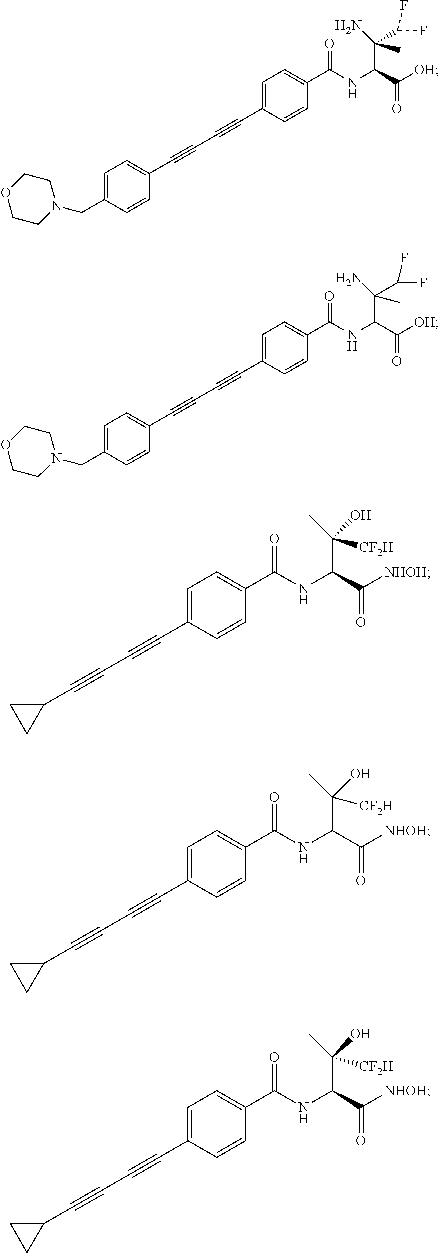

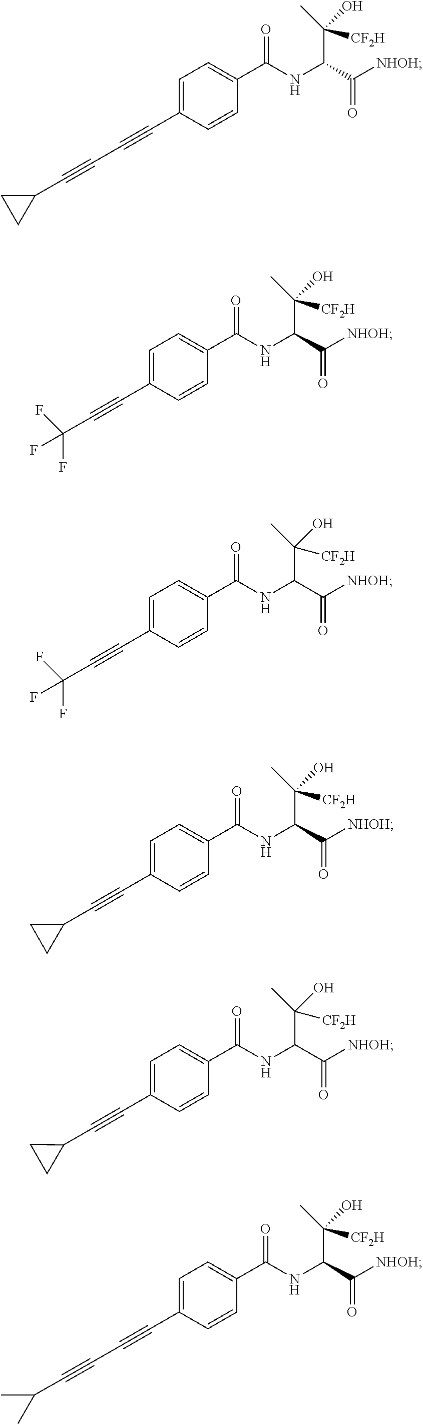

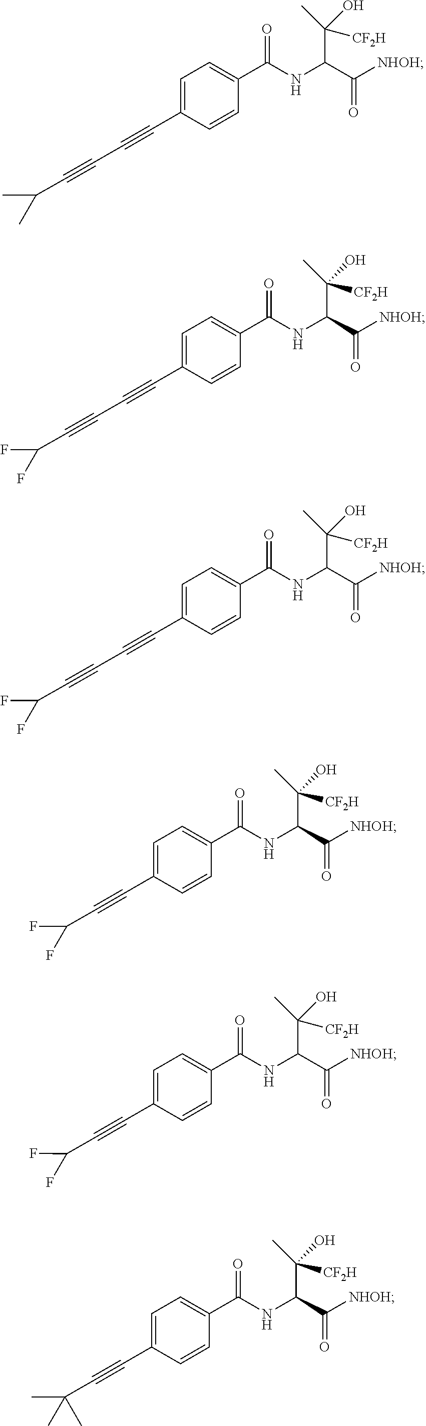

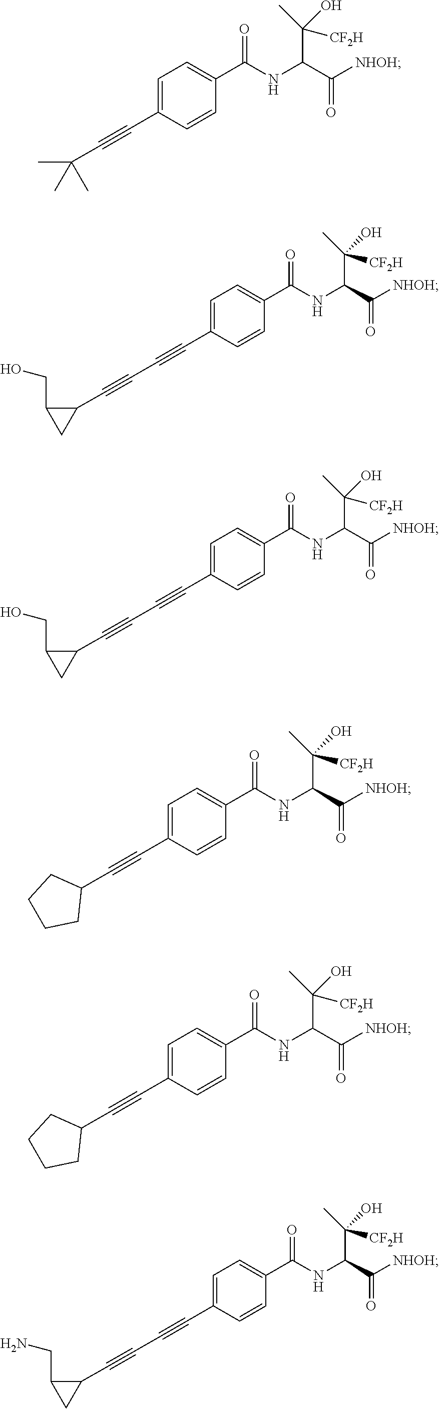

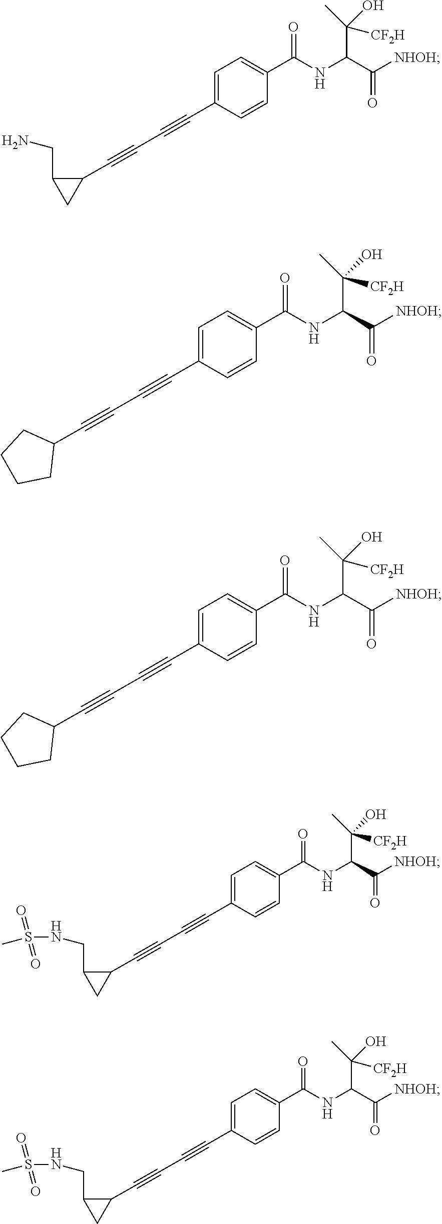

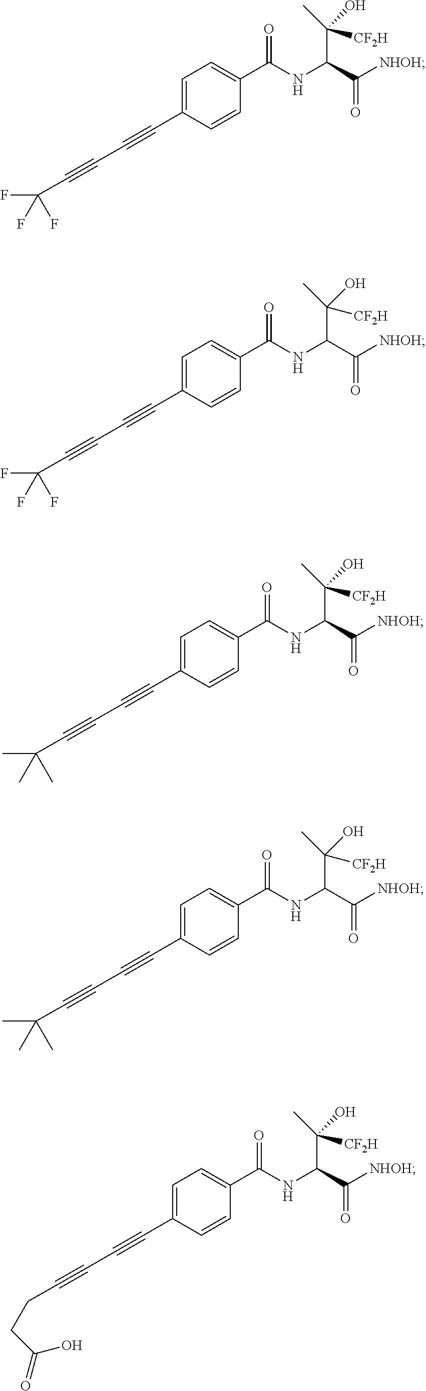

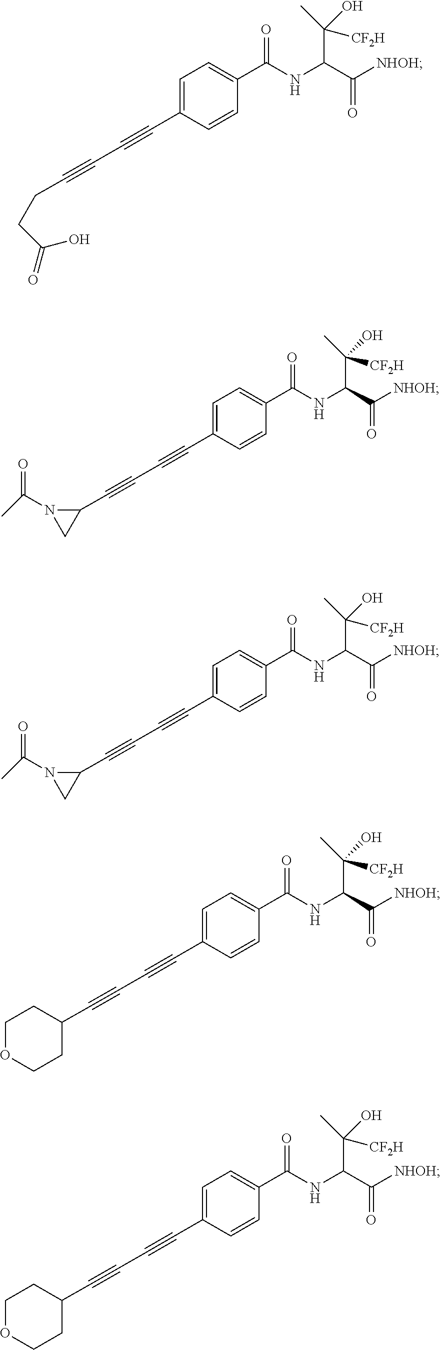

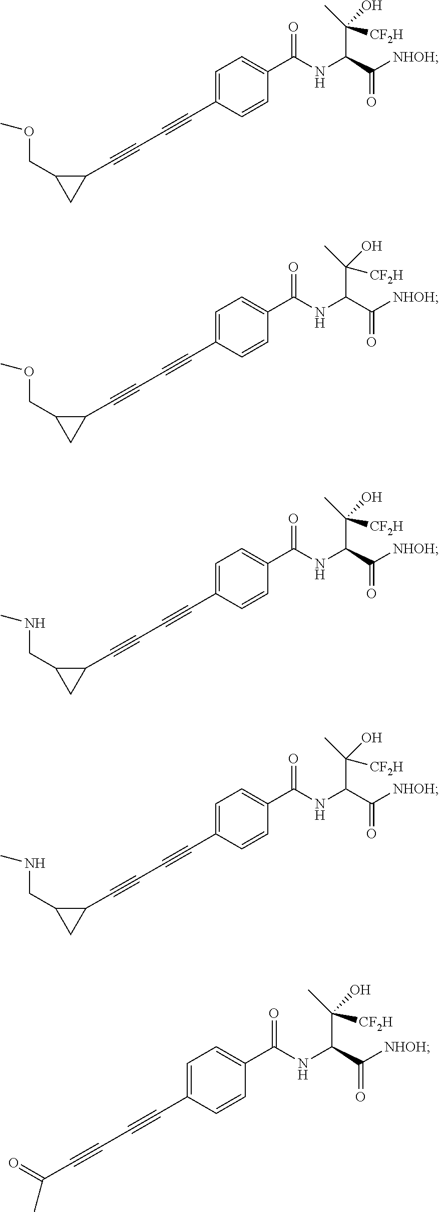

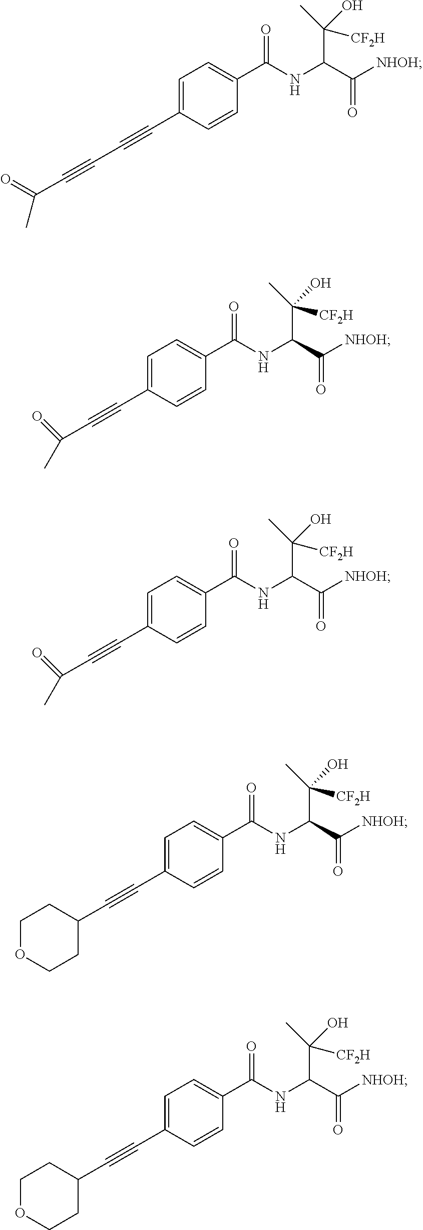

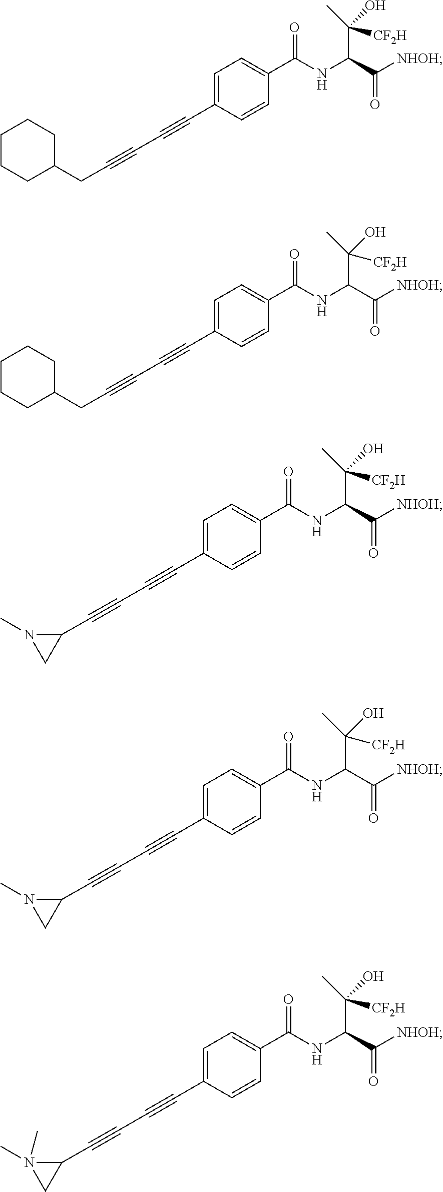

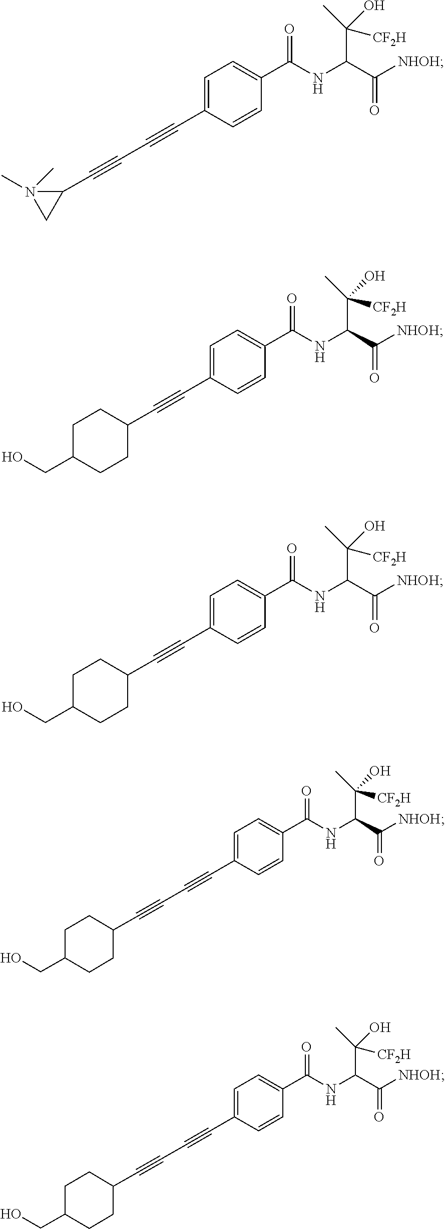

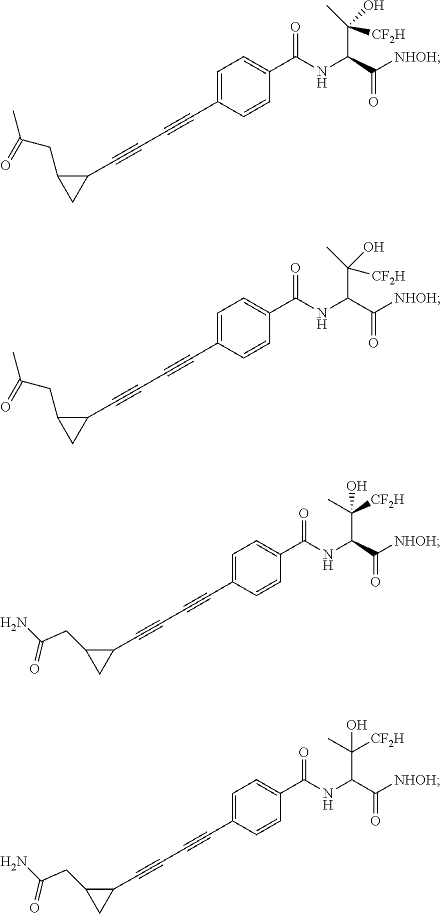

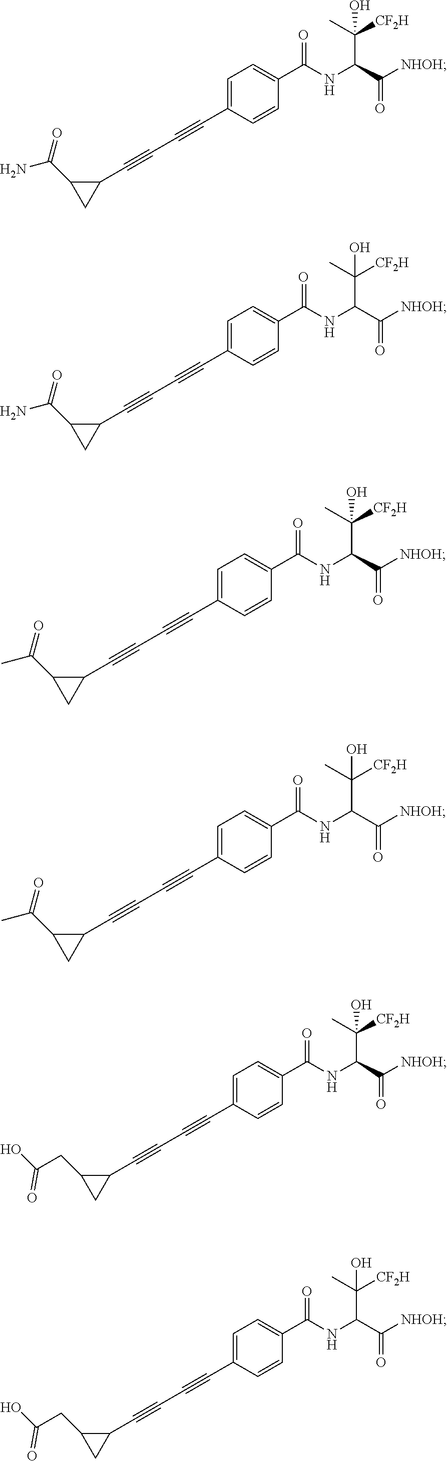

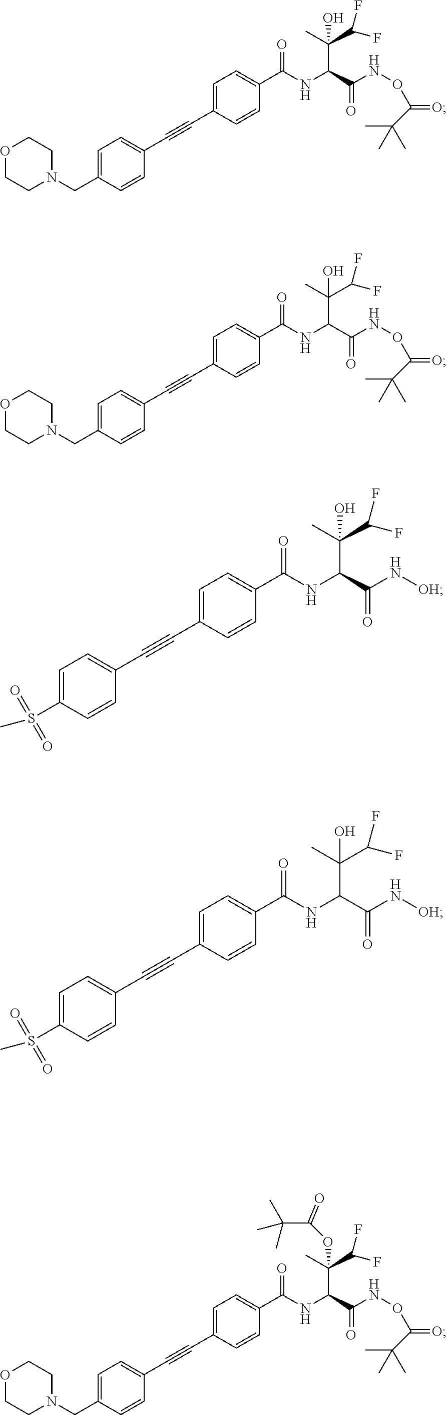 [SMILES: CC(C)(C)C(=O)ONC(=O)[C@@H](NC(=O)c1ccc(C#Cc2ccc(CN3CCOCC3)cc2)cc1)[C@](C)(O)C(F)F ; CC(C)(C)C(=O)ONC(=O)C(NC(=O)c1ccc(C#Cc2ccc(CN3CCOCC3)cc2)cc1)C(C)(O)C(F)F ; C[C@@](O)(C(F)F)[C@H](NC(=O)c1ccc(C#Cc2ccc(S(C)(=O)=O)cc2)cc1)C(=O)NO ; CC(O)(C(F)F)C(NC(=O)c1ccc(C#Cc2ccc(S(C)(=O)=O)cc2)cc1)C(=O)NO ; CC(C)(C)C(=O)ONC(=O)[C@@H](NC(=O)c1ccc(C#Cc2ccc(CN3CCOCC3)cc2)cc1)[C@](C)(OC(=O)C(C)(C)C)C(F)F]

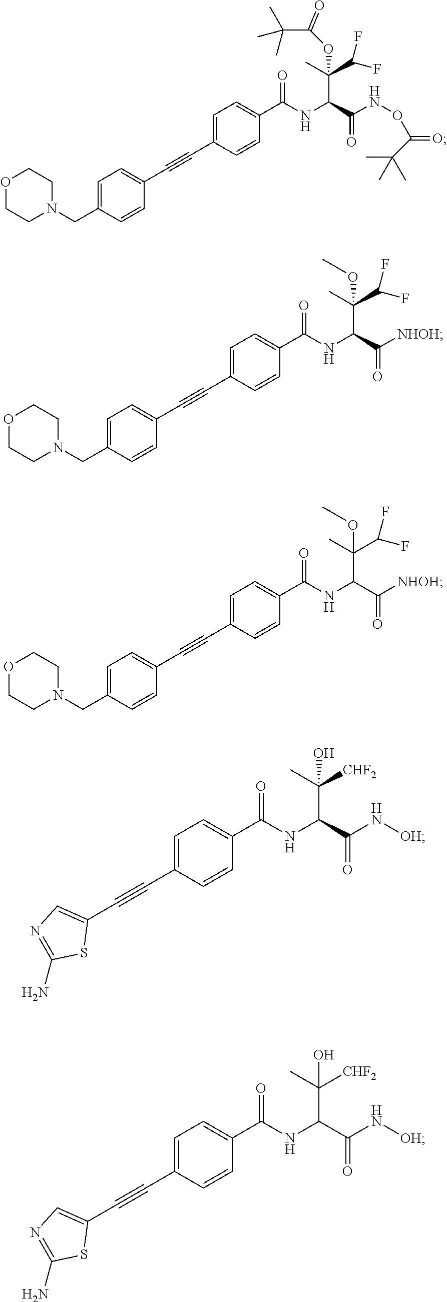

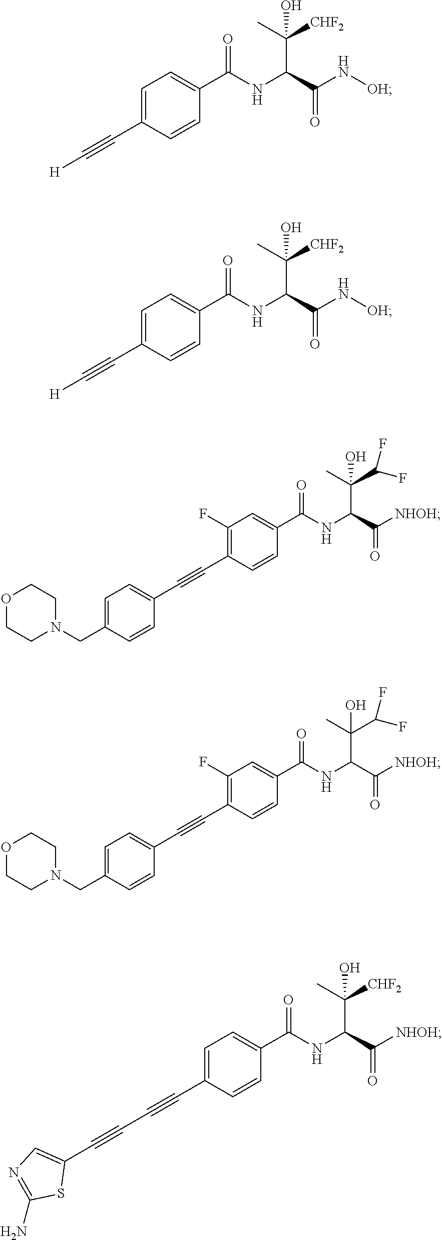

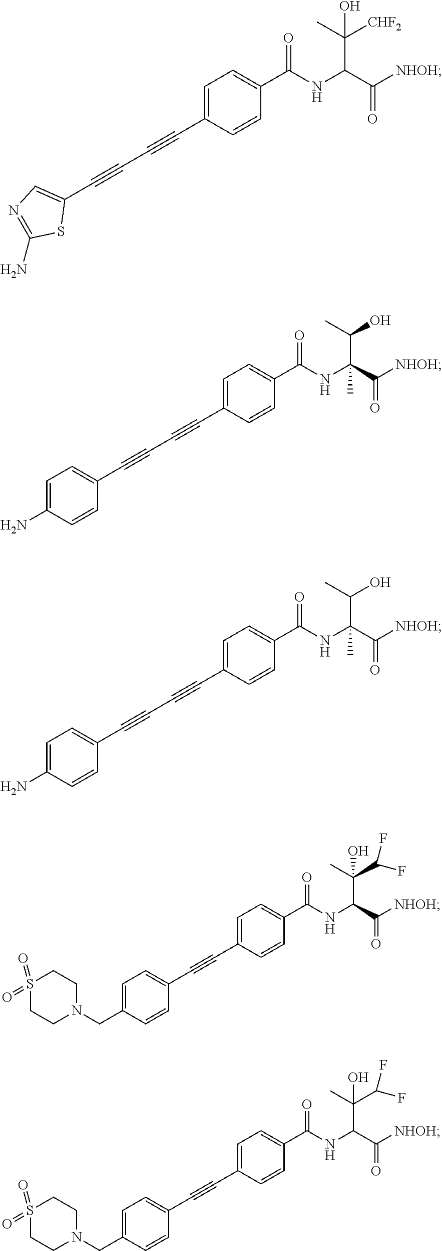

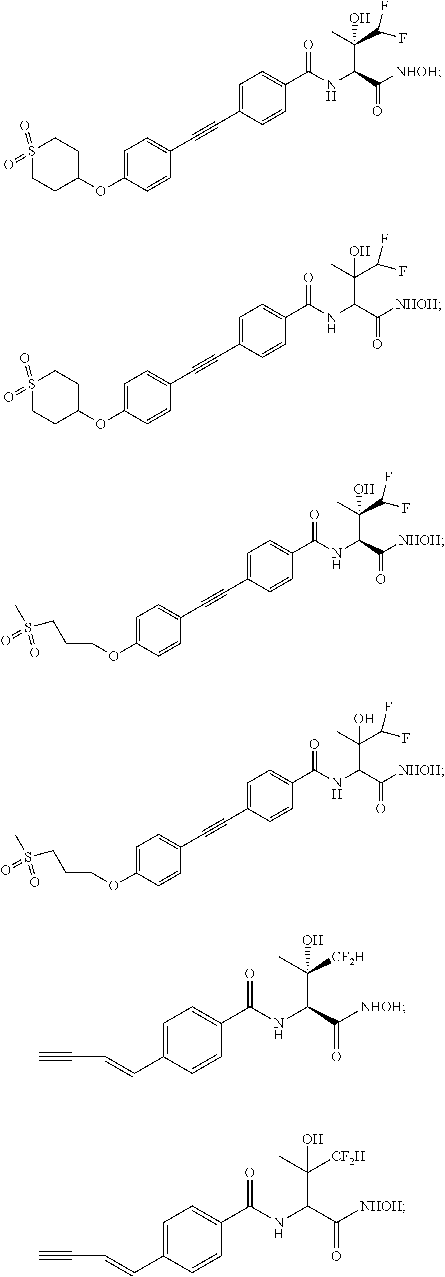

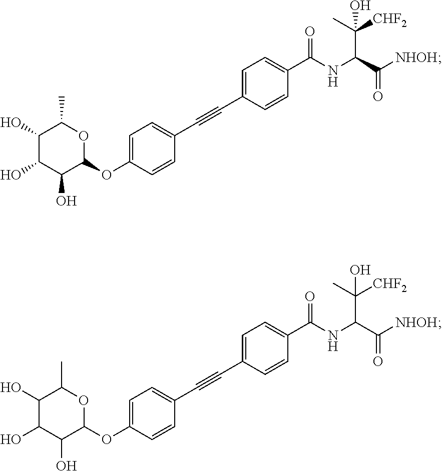

or a stereo-isomer, an enantiomer, a polymorph, a pharmaceutically acceptable salt, an ester, or a derivative thereof.

The inventors have surprisingly discovered that the phosphate pro-drug compounds described herein have greatly increased solubility in a vehicle as compared to their drug counterparts. Specifically, the solubility of a phosphate pro-drug compound described herein in a cyclodextrin vehicle may be increased 2-fold, 4-fold, 6-fold, 8-fold, 10-fold, 12-fold, or 13-fold as compared to the drug counterpart of the phosphate pro-drug compound. In an exemplary embodiment, the cyclodextrin vehicle comprises Captisol®.

### C. Pharmaceutical Compositions

In another aspect, the present disclosure provides compositions comprising one or more of compounds as described herein and an appropriate vehicle, excipient, adjuvant or diluent. The exact nature of the vehicle, excipient or diluent will depend upon the desired use for the composition, and may range from being suitable or acceptable for veterinary uses to being suitable or acceptable for human use. The composition may optionally include one or more additional compounds.

In some embodiments, the pharmaceutical compositions provided herein further comprise a vehicle. In some embodiments, the vehicle is a cyclodextrin, such as α-cyclodextrin, 3-cyclodextrin, γ-cyclodextrin, a derivative thereof or a combination thereof. In one embodiment, the vehicle is a sulfo-butyl-ether β-cyclodextrin, such as Captisol®. In some embodiments, the vehicle is at a concentration from 25-400 mg/mL, 25-300 mg/mL, 25-200 mg/mL, 25-100 mg/mL, 25-50 mg/mL, 50-400 mg/mL, 50-300 mg/mL, 60-400 mg/mL, 60-300 mg/mL, 150-400 mg/mL, 150-300 mg/mL, 200-300 mg/mL, 200-400 mg/mL; 30-100 mg/mL, 300-400 mg/mL, 30-100 mg/mL, 45-75 mg/mL, 50-70 mg/mL, 55-65 mg/mL, or 50-60 mg/mL. In another embodiment, pharmaceutical composition comprises a vehicle at a concentration from 2.5-40% (w/v), 2.5-30% (w/v), 2.5-20% (w/v), 2.5-10% (w/v), 5-40% (w/v), 5-30% (w/v), 5-20% (w/v), 5-10% (w/v), 6-40% (w/v), 6-30% (w/v), 6-20% (w/v), 6-10% (w/v), 10-40% (w/v), 10-30% (w/v), 10-20% (w/v), 20-40% (w/v), 20-30% (w/v), 25-40% (w/v), 25-30% (w/v), 3-10% (w/v), 4.5-7.5% (w/v), 5-7% (w/v), 5.5-6.5% (w/v) of the vehicle. In some embodiments, the pharmaceutical composition comprises 2.5% (w/v), 3% (w/v), 4% (w/v), 4.5% (w/v), 5% (w/v), 5.5% (w/v), 6% (w/v), 6.5% (w/v), 7% (w/v), 7.5% (w/v), 8% (w/v), 8.5% (w/v), 9% (w/v), 9.5% (w/v), 10% (w/v), 15% (w/v), 20% (w/v), 25% (w/v), 30% (w/v), 35% (w/v) or 40% (w/v) of the vehicle.

When used to treat or prevent a disease, such as a bacterial infection, the compounds described herein may be administered singly, as mixtures of one or more compounds or in mixture or combination with other agents (e.g., therapeutic agents) useful for treating such diseases and/or the symptoms associated with such diseases. Such agents may include, but are not limited to, antibiotics, NSAIDS, anti-inflammatory compounds, chemotherapeutic agents, anticancer drugs, immunotherapy, polyclonal or monoclonal antibodies, non-immune tolerizing approaches, liposome-based cytotoxin inhibitors, to name a few. The compounds may be administered in the form of compounds per se, or as pharmaceutical compositions comprising a compound.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

### D. Methods of Synthesis

The present disclosure further provides methods for synthesizing the compounds provided herein. The compound synthesized may be any of the compounds described in Section B above.

The synthesis of a compound of Formula (I) may comprise
(a) contacting a compound of Formula (I′)

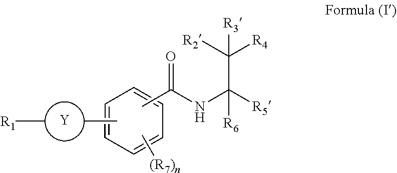

with a phosphate salt of the formula

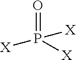

in the presence of a base catalyst to form a reaction mixture, and
(b) adding an alkali metal hydroxide to the reaction mixture to form the compound of Formula (I). The synthesis may be represented by the following reaction scheme:

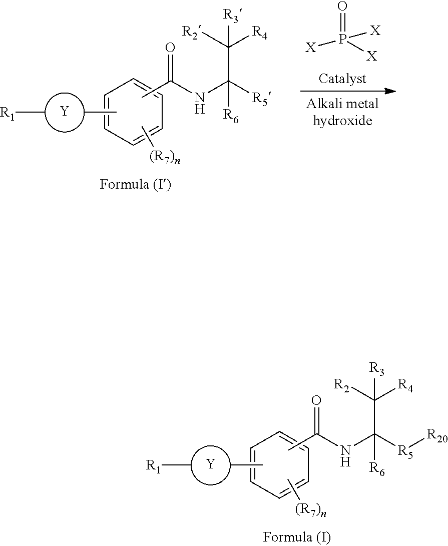

wherein Y represents aryl optionally substituted with R₈, heteroaryl optionally substituted with R₈, cycloalkyl, or heterocyclyl optionally substituted with R₈;
X is a halogen;
n is an integer selected from 0, 1, 2, 3, or 4;
R₁ is hydrogen, C₁-C₆ alkyl optionally substituted with R₉, aryl optionally substituted with R₉, heteroaryl optionally substituted with R₉, or heterocyclyl optionally substituted with R₉;
R₂ is halogen, C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₃ is halogen, C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₃′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₄ is C₁-C₆ haloalkyl;
R₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₅′ is —C₁-C₆ alkyl, —C₁-C₆ haloalkyl, —NH₂, —NH(C₁-C₆ alkyl), —N(C₁-C₆ alkyl)₂, —OH, —C₁-C₆ alkoxy, —C₁-C₆ haloalkoxy-, —CONH, —CONH(C₁-C₆ alkyl), —CON(C₁-C₆ alkyl)₂, —CONH—OH, —CONH(C₁-C₆ alkyl)-OH, —CONH₂, —OCO(C₁-C₆ alkyl), —O—C(C₁-C₆ alkyl)₂, —CONH—NH₂, —COOH, or —CO₂(C₁-C₆ alkyl);
R₆ is hydrogen or C₁-C₆ alkyl;
each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups that when attached to the same carbon atom form ═O;
each R₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups that when attached to the same carbon atom form ═O; and
each R₂₀ is independently of Formula (IIa):

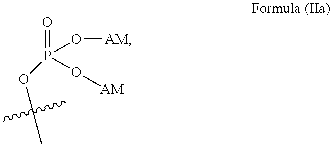

wherein each AM is independently selected from Na, Li, K, Rb, or a C₁-C₆ alkyl. In an exemplary embodiment, each AM is independently Na or methyl. In another exemplary embodiment, each X is independently Cl. It should be understood that the compound of Formula (I) may be any compound of Formula (I) described in Section B.

The reaction mixture may be mixed to ensure sufficient contact of the reactants to form the phosphate prodrug compound. Methods and apparatuses for mixing are generally well known to those having ordinary skill in the art.

The base catalyst may comprise a cyclic tertiary amine, such as a morpholine, a piperazine, a piperidine, a hydroxypiperidine, a halopiperidine, a pyrrolidine, a N-alkyltetrahydroquinolone, and the like. In an exemplary embodiment, the cyclic tertiary amine may be N-methylmorpholine. In another embodiment, the base may comprise sodium carbonate, potassium carbonate, cesium carbonate, sodium hydrogen carbonate, potassium hydrogen carbonate, sodium acetate, potassium acetate, potassium hydroxide, sodium hydroxide, lithium hydroxide, sodium amide, sodium methoxide, potassium t-butoxide, sodium hydride, lithium hydride, triethylamine, diisopropylethylamine, dimethylaniline or a combination thereof. The base catalyst may be present in the reaction mixture in a molar excess as compared to the compound of Formula (I′). In an exemplary embodiment, the molar ratio of the base catalyst to the compound of Formula (I′) in the reaction mixture is about 5:1.

The alkali metal hydroxide may comprise lithium hydroxide, sodium hydroxide, potassium hydroxide, cesium hydroxide, or rubidium hydroxide. In an exemplary embodiment, the alkali metal hydroxide comprises sodium hydroxide.

The method may be conducted at a temperature from about 0° C. to about −20° C. Accordingly, step (a) or step (b) may be conducted at a temperature from about 0° C. to about −20° C. In an exemplary embodiment the contacting step is accomplished in an ice-NaCl bath.

The method may be conducted at atmospheric pressure, under vacuum, or under pressurized conditions.

The method may be conducted in an inert atmosphere or under an ambient atmosphere.

The compound of Formula (I′) may be present in a stoichiometric equivalent amount as compared to the phosphate salt. In some embodiments, the compound of Formula (IF) may be present in the reaction mixture in a molar excess as compared to the phosphate salt. In some other embodiments, the phosphate salt may be present in the reaction mixture in a molar excess as compared to the compound of Formula (I).

The step of contacting a compound of Formula (I′) with the phosphate salt in the presence of a base catalyst to form the reaction mixture may be performed in the presence of an organic solvent. The organic solvent may be a nonpolar, weakly polar, or a polar solvent. In some embodiments, the organic solvent includes acetonitrile, acetone, methanol, ethanol, isopropanol, chloroform, methylene chloride, dichloroethane, carbon tetrachloride, diethyl ether, diisopropyl ether, tetrahydrofuran, 1,4-dioxane, dimethoxyethane, dimethylformamide, dimethyl sulfoxide, ethyl acetate, t-butyl acetate, propionitrile, benzene, toluene, anisole, cyclohexane, or a combination thereof. In an exemplary embodiment, the organic solvent includes acetonitrile.

The synthesis of a compound of Formula (IV) may comprise
(a) contacting a compound of Formula (IV′)

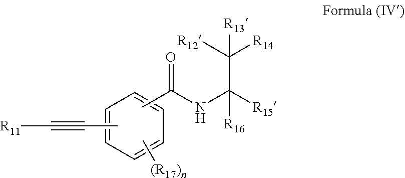

with a phosphate salt of the formula

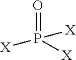

in the presence of a base catalyst to form a reaction mixture, and
(b) adding an alkali metal hydroxide to the reaction mixture to form the compound of Formula (IV). The synthesis may be represented by the following reaction scheme:

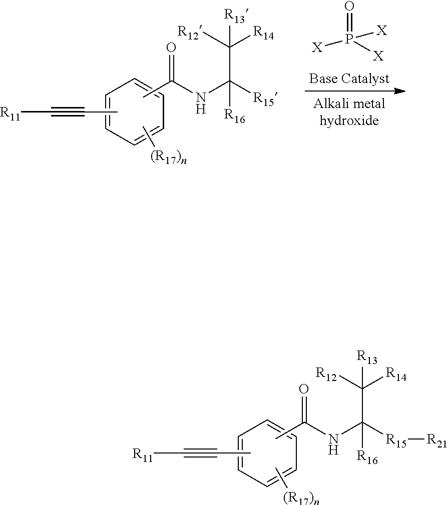

X is a halogen;
n is an integer selected from 0, 1, 2, 3, or 4;
R₁₁ is —C≡C—R₃₃, C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈;
wherein R₃₃ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₉, (C₃-C₈ cycloalkyl) C₁-C₆ alkyl-optionally substituted with R₁₉, a C₃-C₇ cyclyl or heterocyclyl optionally substituted with R₁₉;
R₁₂ is halogen, C₁-C₆ alkyl, C₁-C₆haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₂′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₃ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₃′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₄ is C₁-C₆ haloalkyl;
R₁₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₁₅′ is —C₁-C₆ alkyl, —C₁-C₆ haloalkyl, —NH₂, —NH(C₁-C₆ alkyl), —NH(C₁-C₆ alkyl), —N(C₁-C₆ alkyl)₂, —OH, —C₁-C₆ alkoxy, —C₁-C₆ haloalkoxy, —CONH₂, —CONH(C₁-C₆ alkyl), —CON(C₁-C₆ alkyl), —CON(C₁-C₆ alkyl)₂, —CONH—OH, —CON(C₁-C₆ alkyl)-OH, —CONH₂, —OCO(C₁-C₆ alkyl), —O—CH(C₁-C₆ alkyl)₂, —CONH—NH₂, —CO₂H, or —CO₂(C₁-C₆ alkyl);
R₁₆ is hydrogen or C₁-C₆ alkyl;
each R₁₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₁₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups that when attached to the same carbon atom form ═O;
each R₁₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₁₉ groups that when attached to the same carbon atom form ═O; and
each R₂₁ is independently of Formula (IIa):

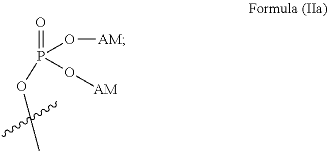

wherein each AM is independently selected from Na, Li, K, Rb, or a C₁-C₆ alkyl. In an exemplary embodiment, each AM is independently Na or methyl. In another exemplary embodiment, each X is independently Cl. It should be understood that the compound of Formula (IV) may be any compound of Formula (IV) described in Section B.

The reaction mixture may be mixed to ensure sufficient contact of the reactants to form the phosphate prodrug compound. Methods and apparatuses for mixing are generally well known to those having ordinary skill in the art.

The base catalyst may comprise a cyclic tertiary amine, such as a morpholine, a piperazine, a piperidine, a hydroxypiperidine, a halopiperidine, a pyrrolidine, a N-alkyltetrahydroquinolone, and the like. In an exemplary embodiment, the cyclic tertiary amine may be N-methylmorpholine. In another embodiment, the base may comprise sodium carbonate, potassium carbonate, cesium carbonate, sodium hydrogen carbonate, potassium hydrogen carbonate, sodium acetate, potassium acetate, potassium hydroxide, sodium hydroxide, lithium hydroxide, sodium amide, sodium methoxide, potassium t-butoxide, sodium hydride, lithium hydride, triethylamine, diisopropylethylamine, dimethylaniline or a combination thereof. The base catalyst may be present in the reaction mixture in a molar excess as compared to the compound of Formula (IV′). In an exemplary embodiment, the molar ratio of the base catalyst to the compound of Formula (IV′) in the reaction mixture is about 5:1.

The alkali metal hydroxide may comprise lithium hydroxide, sodium hydroxide, potassium hydroxide, cesium hydroxide, or rubidium hydroxide. In an exemplary embodiment, the alkali metal hydroxide comprises sodium hydroxide.

The method may be conducted at a temperature from about 0° C. to about −20° C. Accordingly, step (a) or step (b) may be conducted at a temperature from about 0° C. to about −20° C. In an exemplary embodiment the contacting step is accomplished in an ice-NaCl bath.

The method may be conducted at atmospheric pressure, under vacuum, or under pressurized conditions.

The method may be conducted in an inert atmosphere or under an ambient atmosphere.

The compound of Formula (IV′) may be present in a stoichiometric equivalent amount as compared to the phosphate salt. In some embodiments, the compound of Formula (IV′) may be present in the reaction mixture in a molar excess as compared to the phosphate salt. In some other embodiments, the phosphate salt may be present in the reaction mixture in a molar excess as compared to the compound of Formula (IV).

The step of contacting a compound of Formula (IV′) with the phosphate salt in the presence of a base catalyst to form the reaction mixture may be performed in the presence of an organic solvent. The organic solvent may be a nonpolar, weakly polar, or a polar solvent. In some embodiments, the organic solvent includes acetonitrile, acetone, methanol, ethanol, isopropanol, chloroform, methylene chloride, dichloroethane, carbon tetrachloride, diethyl ether, diisopropyl ether, tetrahydrofuran, 1,4-dioxane, dimethoxyethane, dimethylformamide, dimethyl sulfoxide, ethyl acetate, t-butyl acetate, propionitrile, benzene, toluene, anisole, cyclohexane, or a combination thereof. In an exemplary embodiment, the organic solvent includes acetonitrile.

The synthesis of a compound of Formula (VI) may comprise
(a) contacting a compound of Formula (VI′)

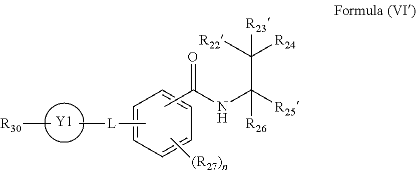

with a phosphate salt of the formula

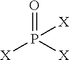

in the presence of a base catalyst to form a reaction mixture, and
(b) adding an alkali metal hydroxide to the reaction mixture to form the compound of Formula (IV). The synthesis may be represented by the following reaction scheme:

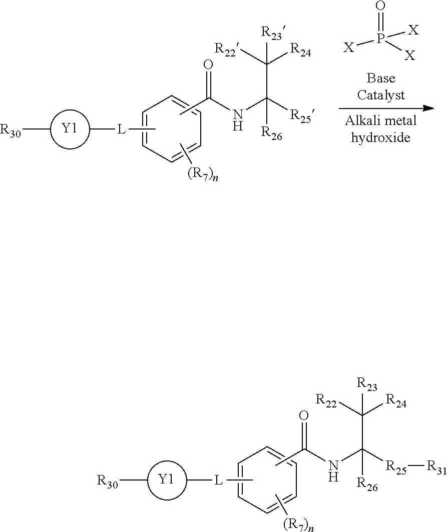

X is a halogen;
L represents a linker, wherein L is absent or is selected from the group consisting of —C≡C—C≡C—, or —C≡C—R₃₂, wherein R₃₂ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₂₉, (C₃-C₈ cycloalkyl) C₁-C₆ alkyl- optionally substituted with R₂₉, a C₃-C₇ cyclyl and heterocyclyl optionally substituted with R₂₉;
Y1 represents aryl optionally substituted with R₂₈, heteroaryl optionally substituted with R₂₈, cycloalkyl, or heterocyclyl optionally substituted with R₂₈;
n is an integer selected from 0, 1, 2, 3, or 4;
R₃₀ is hydrogen, C₁-C₆ alkyl optionally substituted with R₂₉, aryl optionally substituted with R₂₉, heteroaryl optionally substituted with R₂₉, or heterocyclyl optionally substituted with R₂₉;
R₂₂ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₂′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₃ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₃′ is halogen, C₁-C₆ alkyl, C₁-C₆ haloalkyl, —NH₂, —NH(C₁-C₆ alkyl), —N(C₁-C₆ alkyl)₂, —OH, —C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₄ is C₁-C₆ haloalkyl;
R₂₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₂₅′ is —C₁-C₆ alkyl, —C₁-C₆ haloalkyl, —NH₂, —NH(C₁-C₆ alkyl), —NH(C₁-C₆ alkyl), —N(C₁-C₆ alkyl)₂, —OH, —C₁-C₆ alkoxy, —C₁-C₆ haloalkoxy, —CONH₂, —CONH(C₁-C₆ alkyl), —CON(C₁-C₆ alkyl)₂, —CONH—OH, —CONH(C₁-C₆ alkyl)-OH, —CONH₂, —OCO(C₁-C₆ alkyl), —O—CH(C₁-C₆ alkyl)₂, —CONH—NH₂, —CO₂H, or —CO₂(C₁-C₆ alkyl);
R₂₆ is hydrogen or C₁-C₆ alkyl;
each R₂₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
each R₃₁ is independently a compound of Formula (IIa):

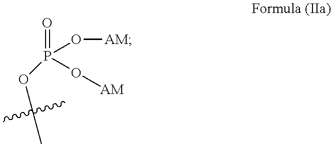

wherein each AM is independently selected from Na, Li, K, Rb, or a C₁-C₆ alkyl. In an exemplary embodiment, each AM is independently Na or methyl. In another exemplary embodiment, each X is independently Cl. It should be understood that the compound of Formula (VI) may be any compound of Formula (VI) described in Section B.

The reaction mixture may be mixed to ensure sufficient contact of the reactants to form the phosphate prodrug compound. Methods and apparatuses for mixing are generally well known to those having ordinary skill in the art.

The base catalyst may comprise a cyclic tertiary amine, such as a morpholine, a piperazine, a piperidine, a hydroxypiperidine, a halopiperidine, a pyrrolidine, a N-alkyltetrahydroquinolone, and the like. In an exemplary embodiment, the cyclic tertiary amine may be N-methylmorpholine. In another embodiment, the base may comprise sodium carbonate, potassium carbonate, cesium carbonate, sodium hydrogen carbonate, potassium hydrogen carbonate, sodium acetate, potassium acetate, potassium hydroxide, sodium hydroxide, lithium hydroxide, sodium amide, sodium methoxide, potassium t-butoxide, sodium hydride, lithium hydride, triethylamine, diisopropylethylamine, dimethylaniline or a combination thereof. The base catalyst may be present in the reaction mixture in a molar excess as compared to the compound of Formula (VI′). In an exemplary embodiment, the molar ratio of the base catalyst to the compound of Formula (VI′) in the reaction mixture is about 5:1.

The alkali metal hydroxide may comprise lithium hydroxide, sodium hydroxide, potassium hydroxide, cesium hydroxide, or rubidium hydroxide. In an exemplary embodiment, the alkali metal hydroxide comprises sodium hydroxide.

The method may be conducted at a temperature from about 0° C. to about −20° C. Accordingly, step (a) or step (b) may be conducted at a temperature from about 0° C. to about −20° C. In an exemplary embodiment the contacting step is accomplished in an ice-NaCl bath.

The method may be conducted at atmospheric pressure, under vacuum, or under pressurized conditions.

The method may be conducted in an inert atmosphere or under an ambient atmosphere.

The compound of Formula (VI′) may be present in a stoichiometric equivalent amount as compared to the phosphate salt. In some embodiments, the compound of Formula (VI′) may be present in the reaction mixture in a molar excess as compared to the phosphate salt. In some other embodiments, the phosphate salt may be present in the reaction mixture in a molar excess as compared to the compound of Formula (VI).

The step of contacting a compound of Formula (VI′) with the phosphate salt in the presence of a base catalyst to form the reaction mixture may be performed in the presence of an organic solvent. The organic solvent may be a nonpolar, weakly polar, or a polar solvent. In some embodiments, the organic solvent includes acetonitrile, acetone, methanol, ethanol, isopropanol, chloroform, methylene chloride, dichloroethane, carbon tetrachloride, diethyl ether, diisopropyl ether, tetrahydrofuran, 1,4-dioxane, dimethoxyethane, dimethylformamide, dimethyl sulfoxide, ethyl acetate, t-butyl acetate, propionitrile, benzene, toluene, anisole, cyclohexane, or a combination thereof. In an exemplary embodiment, the organic solvent includes acetonitrile.

The synthesis of a compound of Formula (I) may comprise contacting a compound of Formula (I″)

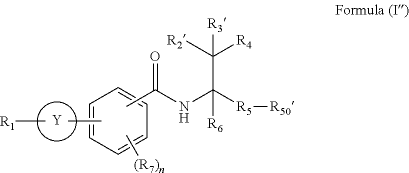

with a) a monophosphate kinase, b) a mixture of H₃PO₄ and P₄O₁₀, c) P₄O₁₀ alone, or d) a mixture of p-toluenesulfonyl chloride (TsCl) and pyridine to form a compound of Formula (T)

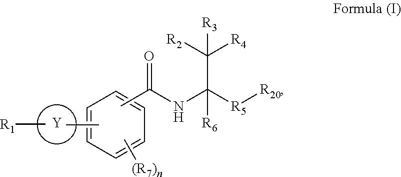

wherein Y represents aryl optionally substituted with R₈, heteroaryl optionally substituted with R₈, cycloalkyl, or heterocyclyl optionally substituted with R₈;
X is a halogen;
n is an integer selected from 0, 1, 2, 3, or 4;
R₁ is hydrogen, C₁-C₆ alkyl optionally substituted with R₉, aryl optionally substituted with R₉, heteroaryl optionally substituted with R₉, or heterocyclyl optionally substituted with R₉;
R₂ is halogen, C₁-C₆ alkyl, C₁-C₆haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₃ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₃′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₄ is C₁-C₆ haloalkyl;
R₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₅₀′ is H or Formula (IIa)

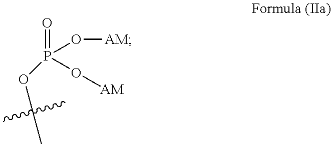

R₆ is hydrogen or C₁-C₆ alkyl;
each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups that when attached to the same carbon atom form ═O;
each R₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups that when attached to the same carbon atom form ═O; and
R₂₀ is of Formula (IIb):

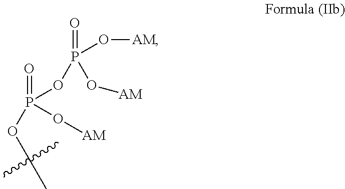

wherein each AM is independently selected from Na, Li, K, Rb, or a C₁-C₆alkyl. In an exemplary embodiment, each AM is independently Na or methyl.

When the method comprises contacting the compound of Formula (I″) with a mixture of H₃PO₄ and P₄O₁₀ or P₄O₁₀ alone, the method may further comprise adding a base to the reaction mixture to give the trisodium salt. In an exemplary embodiment, the base is NaHCO₃. In some embodiments, when the method comprises contacting the compound of Formula (I″) with a mixture of H₃PO₄ and P₄O₁₀ or P₄O₁₀ alone, R₅₀′ is Formula (IIa).

When the method comprises contacting the compound of Formula (I″) with TsCl and pyridine, the method further comprises adding sodium pyrophosphate to the reaction mixture to give the pyrophosphate prodrug. In some embodiments, when the method comprises contacting the compound of Formula (I″) with TsCl and pyridine, R₅₀′ is H.

The synthesis of a compound of Formula (IV) may comprise contacting a compound of Formula (IV″)

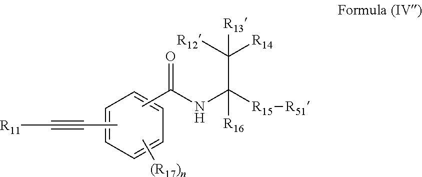

with a) a monophosphate kinase, b) a mixture of H₃PO₄ and P₄O₁₀, c) P₄O₁₀ alone, or d) a mixture of p-toluenesulfonyl chloride (TsCl) and pyridine to form a compound of Formula (IV)

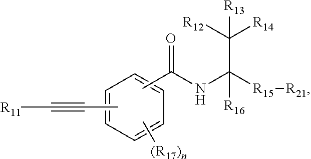

wherein n is an integer selected from 0, 1, 2, 3, or 4;
R₁₁ is —C≡C—R₃₃, C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈;
R₃₃ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₉, (C₃-C₈ cycloalkyl) C₁-C₆ alkyl- optionally substituted with R₁₉, a C₃-C₇ cyclyl or heterocyclyl optionally substituted with R₁₉;
R₁₂ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₂′ is halogen, C₁-C₆ alkyl, C₁-C₆haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₃ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₃′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₄ is C₁-C₆ haloalkyl;
R₅₁′ is H or Formula (IIa)

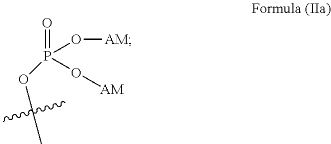

R₁₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₁₆ is hydrogen or C₁-C₆ alkyl;
each R₁₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₁₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups that when attached to the same carbon atom form ═O;
each R₁₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₁₉ groups that when attached to the same carbon atom form ═O; and
R₂₁ is Formula (IIb):

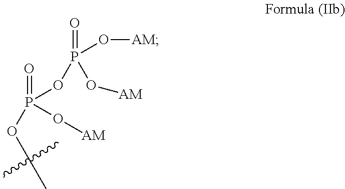

wherein each AM is independently selected from Na, Li, K, Rb, or a C₁-C₆ alkyl. In an exemplary embodiment, each AM is independently Na or methyl.

When the method comprises contacting the compound of Formula (IV″) with a mixture of H₃PO₄ and P₄O₁₀ or P₄O₁₀ alone, the method may further comprise adding a base to the reaction mixture to give the trisodium salt. In an exemplary embodiment, the base is NaHCO₃. In some embodiments when the method comprises contacting the compound of Formula (IV″) with a mixture of H₃PO₄ and P₄O₁₀ or P₄O₁₀ alone, R₅₁′ is Formula (IIa).

When the method comprises contacting the compound of Formula (IV″) with TsCl and pyridine, the method further comprises adding sodium pyrophosphate to the reaction mixture to give the pyrophosphate prodrug. In some embodiments when the method comprises contacting the compound of Formula (IV″) with TsCl and pyridine, R₅₁′ is —H.

The synthesis of a compound of Formula (VI) may comprise contacting a compound of Formula (VI″)

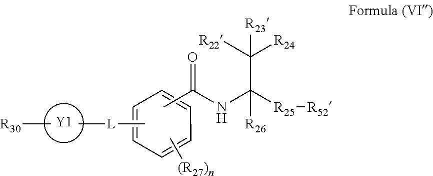

with a) a monophosphate kinase, b) a mixture of H₃PO₄ and P₄O₁₀, c) P₄O₁₀ alone, or d) a mixture of p-toluenesulfonyl chloride (TsCl) and pyridine to form a compound of Formula (VI)

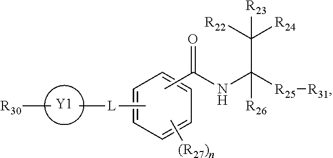

wherein
L represents a linker, wherein L is absent or is selected from the group consisting of —C≡C—C≡C—, or —C≡C—R₃₂, wherein R₃₂ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₂₉, (C₃-C₈ cycloalkyl) C₁-C₆ alkyl- optionally substituted with R₂₉, a C₃-C₇ cyclyl and heterocyclyl optionally substituted with R₂₉;
Y1 represents aryl optionally substituted with R₂₈, heteroaryl optionally substituted with R₂₈, cycloalkyl, or heterocyclyl optionally substituted with R₂₈;
n is an integer selected from 0, 1, 2, 3, or 4;
R₃₀ is hydrogen, C₁-C₆ alkyl optionally substituted with R₂₉, aryl optionally substituted with R₂₉, heteroaryl optionally substituted with R₂₉, or heterocyclyl optionally substituted with R₂₉;
R₂₂ is halogen, C₁-C₆ alkyl, C₁-C₆haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₂′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₃ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₃′ is halogen, C₁-C₆ alkyl, C₁-C₆ haloalkyl, —NH₂, —NH(C₁-C₆ alkyl), —N(C₁-C₆ alkyl)₂, —OH, —C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₄ is C₁-C₆ haloalkyl;
R₂₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₅₂′ is H or Formula (IIa)

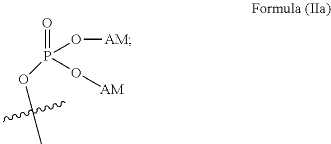

R₂₆ is hydrogen or C₁-C₆ alkyl;
each R₂₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₂₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
each R₃₁ is Formula (IIb);
R₂₀ is of Formula (IIb);

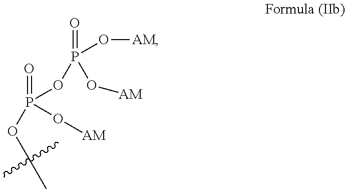

wherein each AM is independently selected from Na, Li, K, Rb, or a C₁-C₆ alkyl. In an exemplary embodiment, each AM is independently Na or methyl.

When the method comprises contacting the compound of Formula (VI″) with a mixture of H₃PO₄ and P₄O₁₀ or P₄O₁₀ alone, the method may further comprise adding a base to the reaction mixture to give the trisodium salt. In an exemplary embodiment, the base is NaHCO₃. In some embodiments when the method comprises contacting the compound of Formula (VI″) with a mixture of H₃PO₄ and P₄O₁₀ or P₄O₁₀ alone, R₅₂′ is Formula (IIa).

When the method comprises contacting the compound of Formula (IV″) with TsCl and pyridine, the method further comprises adding sodium pyrophosphate to the reaction mixture to give the pyrophosphate prodrug. In some embodiments when the method comprises contacting the compound of Formula (IV″) with TsCl and pyridine, R₅₂′ is —H.

### E. Methods of Treatment

The present disclosure further provides methods and/or medicaments for inhibiting deacetylase activities in a subject in need thereof. In one embodiment, the deacetylase is uridyldiphospho-3-O—(R-hydroxydecanoyl)-N-acetylglucosamine deacetylase (LpxC). The gene IpxC encodes the enzyme LpxC. This enzyme is involved in the synthesis of lipid A, the lipid moiety of lipopolysaccharide, which is an essential component of all Gram-negative bacteria. LpxC overexpression may manifest a bacterial infection or a cancer.

In one embodiment, the present disclosure provides a method for treating an infectious disease in a subject, the method comprising, consisting of, or consisting essentially of administering to a subject in need of such treatment an effective amount of a hydroxamate-based phosphate prodrug or a pharmaceutical composition thereof such that the infectious disease is treated in the subject.

In other aspects, the present disclosure provides methods of treating Gram-negative bacterial infections, the method comprising, consisting of, or consisting essentially of administering to a subject in need of such treatment an effective amount of one or more compounds of the present disclosure, or a pharmaceutical composition thereof.

Particular Gram-negative bacteria include, but are not limited to, Pseudomonas aeruginosa, Stenotrophomonas maltophilia, Burkholderia cepacia, Alcaligenes xylosoxidans, Acinetobacter, Enterobacteriaceae, Haemophilus, Neisseria species, Francisella tularensis, Yersinia pestis, Burkholderia pseudomallei, Burkholderia mallei, Rickettsia prowazekii, Coxiella burnetti, Campylobacter jejuni, Shigella, Moraxella catarrhalis, and Chlamydia trachomatis. In one embodiment, the Gram-negative bacteria is Neisseria gonorrhoeae. In some embodiments, the Gram-negative bacteria is Acinetobacter Baumannii.

Specific Enterobacteriaceae include, but are not limited to, Serratia, Proteus, Klebsiella, Enterobacter, Citrobacter, Salmonella, Providencia, Morganella, Cedecea, Edwardsiella, Escherichia coli, Enterobacter cloacae, and Enterobacter aerogenes, and the like.

In another aspect, the present disclosure provides methods for inhibiting a deacetylase enzyme in Gram-negative bacteria, the method comprising, consisting of, or consisting essentially of contacting the bacteria with an effective amount of one or more compounds of the invention. In some embodiments, the deacetylase enzyme comprises LpxC.

The present disclosure further provides methods of treating a cancer in a subject, the method comprising, consisting of, or consisting essentially of administering to a subject in need of such treatment an effective amount of a hydroxamate-based phosphate prodrug or a pharmaceutical composition thereof such that the cancer is treated in the subject.

In some embodiments, the cancer is selected from the group consisting of breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma, peripheral neuroepithelioma, and combinations thereof.

In some embodiments, the methods may further comprise administering to the subject one or more additional therapeutic agents. In one embodiment, the one or more additional therapeutic agent is administered prior to the compound and/or pharmaceutical composition as provided herein. In another embodiment, the one or more additional therapeutic agent is administered concurrently with the compound and/or pharmaceutical composition as provided herein. In another embodiment, the one or more additional therapeutic agent is administered after the compound and/or pharmaceutical composition as provided herein.

### F. Kits

The present disclosure further provides kits comprising the compositions provided herein and for carrying out the subject methods as provided herein. In one embodiment, the kit comprises (1) a container holding the compound and/or composition of the present disclosure, and (2) an instruction for use. In one aspect, the instruction for use comprises steps in administering the compound or the composition of the present disclosure to a subject in need thereof. In another aspect, the subject is a human subject having or is suspect of having a bacterial infection or a cancer. In some embodiments, a kit may further include other components. Such components may be provided individually or in combinations, and may provide in any suitable container such as a vial, a bottle, or a tube. Examples of such components include, but are not limited to, one or more additional reagents, such as one or more dilution buffers; one or more reconstitution solutions; one or more wash buffers; one or more storage buffers, one or more control reagents and the like. Components (e.g., reagents) may also be provided in a form that is usable in a particular assay, or in a form that requires addition of one or more other components before use (e.g. in concentrate or lyophilized form). Suitable buffers include, but are not limited to, phosphate buffered saline, sodium carbonate buffer, sodium bicarbonate buffer, borate buffer, Tris buffer, MOPS buffer, HEPES buffer, and combinations thereof. Kit components may also be provided individually or in combinations, and may be provided in any suitable container, such as a vial, a bottle, or a tube. In some embodiments, the kits disclosed herein comprise one or more reagents for use in the embodiments disclosed herein.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

Another aspect of the present disclosure provides all that is described and illustrated herein.

The following Examples are provided by way of illustration and not by way of limitation.

### EXAMPLES

### Example 1: General Procedure and Study Design

Those having skill in the art will recognize that the starting materials and reaction conditions may be varied, the sequence of the reactions altered, and additional steps employed to produce compounds encompassed by the present disclosure, as demonstrated by the following examples. Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001; or Vogel, A Textbook of Practical Organic Chemistry, Including Qualitative Organic Analysis, Fourth Edition, New York: Longman, 1978).

Starting materials can be obtained from commercial sources or prepared by well-established literature methods known to those of ordinary skill in the art. The reactions are performed in a solvent appropriate to the reagents and materials employed and suitable for the transformations being affected. It will be understood by those skilled in the art of organic synthesis that the functionality present on the molecule should be consistent with the transformations proposed. This will sometimes require a judgment to modify the order of the synthetic steps or to select one particular process scheme over another in order to obtain a desired compound of the disclosure.

In some cases, protection of certain reactive functionalities may be necessary to achieve some of the above transformations. In general, the need for such protecting groups as well as the conditions necessary to attach and remove such groups will be apparent to those skilled in the art of organic synthesis. An authoritative account describing the many alternatives to the trained practitioner are J. F. W. McOmie, “Protective Groups in Organic Chemistry”, Plenum Press, London and New York 1973, in T. W. Greene and P. G. M. Wuts, “Protective Groups in Organic Synthesis”, Third edition, Wiley, New York 1999, in “The Peptides”; Volume 3 (editors: E. Gross and J. Meienhofer), Academic Press, London and New York 1981, in “Methoden der organischen Chemie”, Houben-Weyl, 4.sup.th edition, Vol. 15/1, Georg Thieme Verlag, Stuttgart 1974, in H.-D. Jakubke and H. Jescheit, “Aminosauren, Peptide, Proteine”, Verlag Chemie, Weinheim, Deerfield Beach, and Basel 1982, and/or in Jochen Lehmann. “Chemie der Kohlenhydrate: Monosaccharide and Derivate”, Georg Thieme Verlag, Stuttgart 1974. The protecting groups may be removed at a convenient subsequent stage using methods known from the art.

### Example 2: Synthesis of LPC-233-Phosphate ProDrug

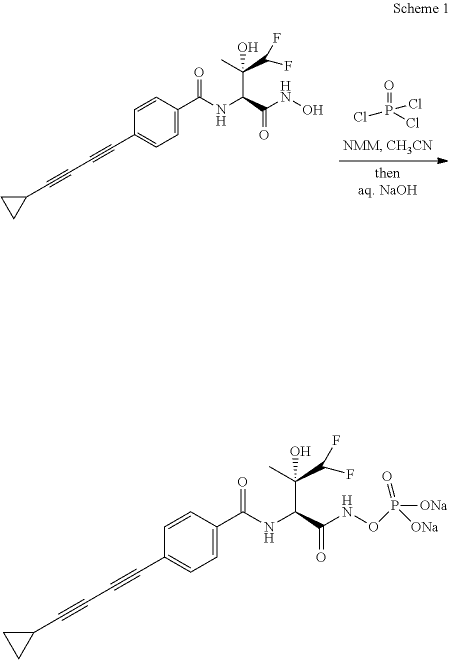

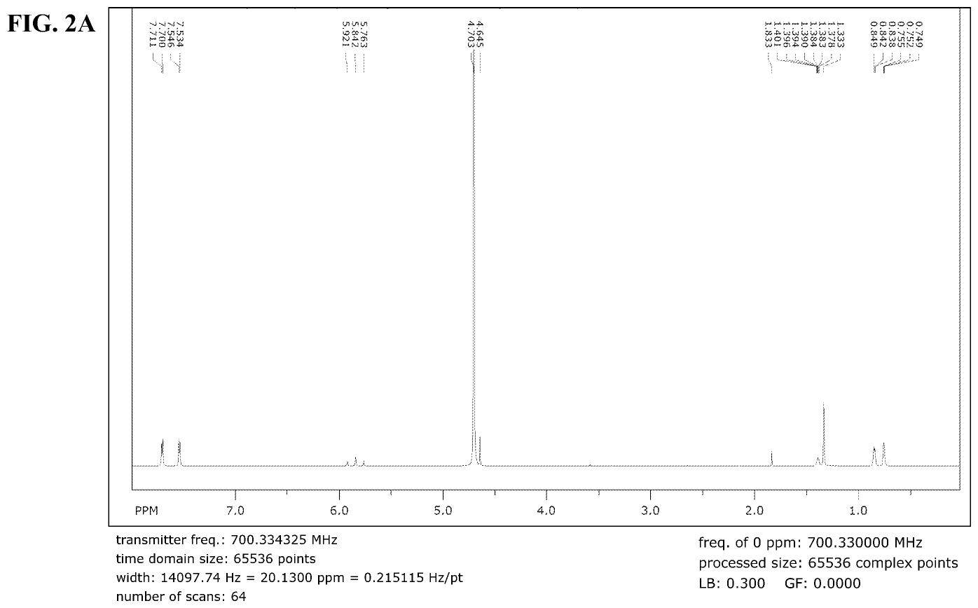

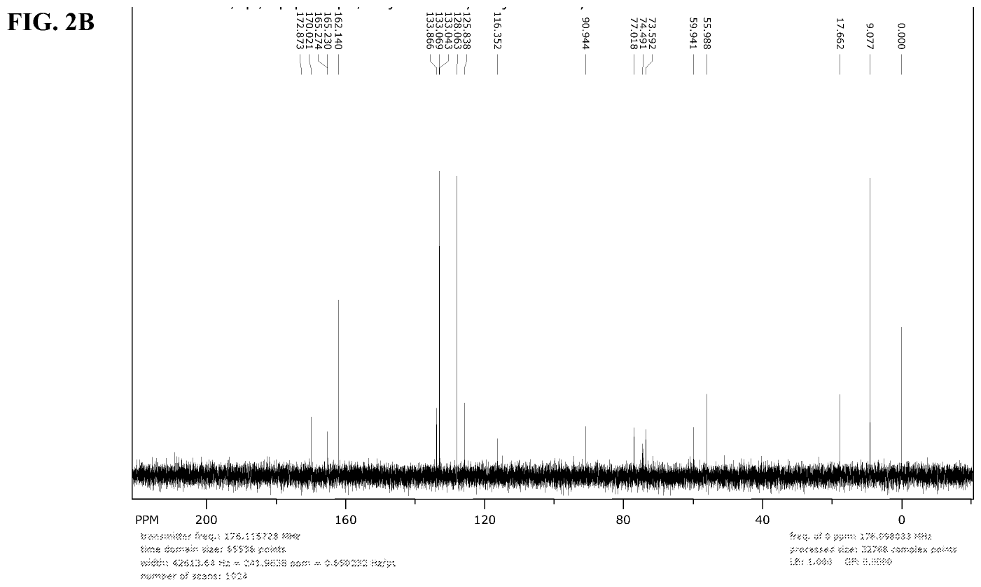

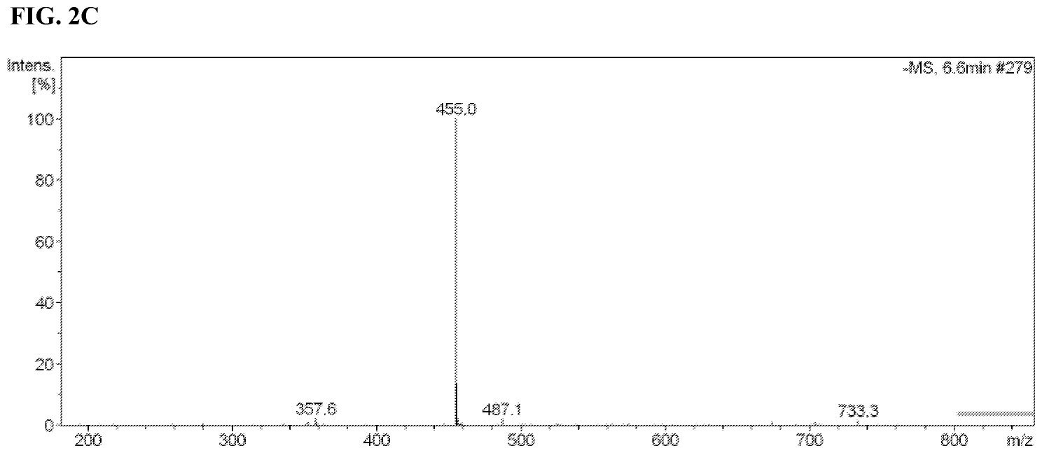

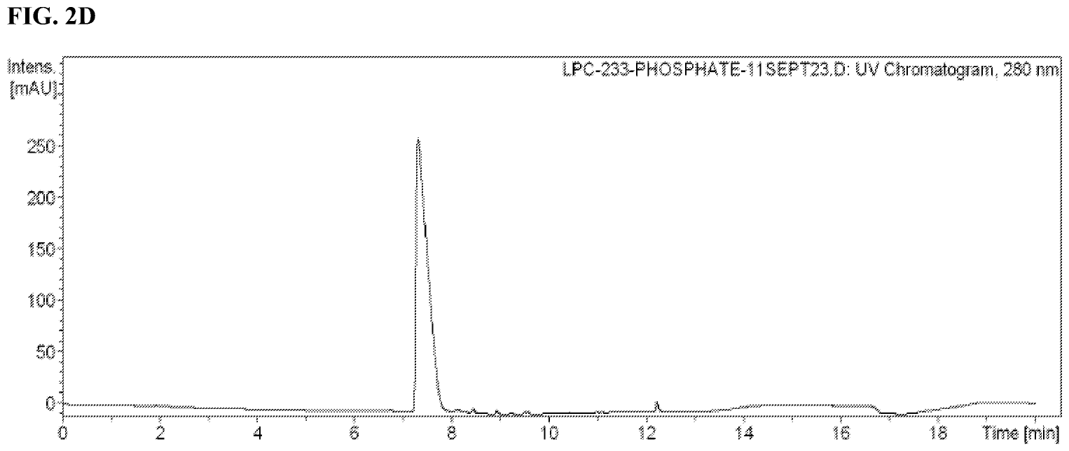

LPC-233-Phosphate ProDrug, also called “LPC-233-Phos-Pro” was synthesized according to Scheme 1. Neat POCl₃ (0.54 mL, 5.6 mmol) was added over 30 seconds to an ice-NaCl bath cooled solution of the hydroxamic acid (2.1 g, 5.6 mmol) and N-Methylmorpholine (NMM) (3.8 mL, 28 mmol, 5 eq.) in CH₃CN (20 mL). The reaction mixture was stirred for an additional 5 minutes then was quenched with one portion of H₂O (20 mL). The entire reaction mixture was concentrated to dryness then redissolved in CH₃CN (125 mL). The resulting solution was treated by dropwise addition of aqueous 4M NaOH (30 mL) over 2-3 minutes. The mixture was transferred to a dropping addition funnel and was allowed to settle. A definite bilayer was observed: a clear amber lower phase and a hazy, translucent upper phase. The lower phase was added [addition was stopped once the phase boundary was reached] dropwise with stirring into acetone (600 mL). This gave a straw-colored sludge and a hazy supernatant. The supernatant was decanted, and an additional portion of acetone (600 mL) was added to the sludge. The mixture was stirred for a few minutes. This transformed the sludge into a granular solid. Insoluble portion was removed by filtration. The filter cake was washed with EtOH (100 mL) and dried overnight. The dry filter cake was dissolved in H₂O (˜5 mL) and loaded directly onto a RediSepRf, C18, 150 g column. Gradient elution from 100% H₂O to 50% MeOH in H₂O over 30 minutes gave the product as a pale faint yellow granular solid (1.0 g, 36%). ESIMS: m/z=455 [(M−2Na)−]. Characterization of LPC-233-Phosphate ProDrug was given in FIGS. 2A-FIG. 2D. This scheme may synthesize LPC-233-Phosphate ProDrug up to 20 gram.

LPC-233 required 30-40% Captisol or beta-cyclodextrin to reach the dose level of 100 mg/kg and beyond. These concentrations of vehicle caused vehicle-related side effects and cannot be used in clinical formulations. The use of the phosphate prodrug enabled us to deliver 100 mg/kg drug at 5% Captisol or beta-cyclodextrin, hence mitigating the vehicle related toxicity.

### Example 3: Synthesis of Pyrophosphate Pro-Drug Compounds

Pathways for the synthesis of the LPC-233 pyrophosphate prodrug compounds of the present disclosure are proposed herein.

In a first pathway, LPC-233-Phos-Pro is reacted with a monophosphate kinase to give the pyrophosphate LPC-233 prodrug according to the following reaction scheme:

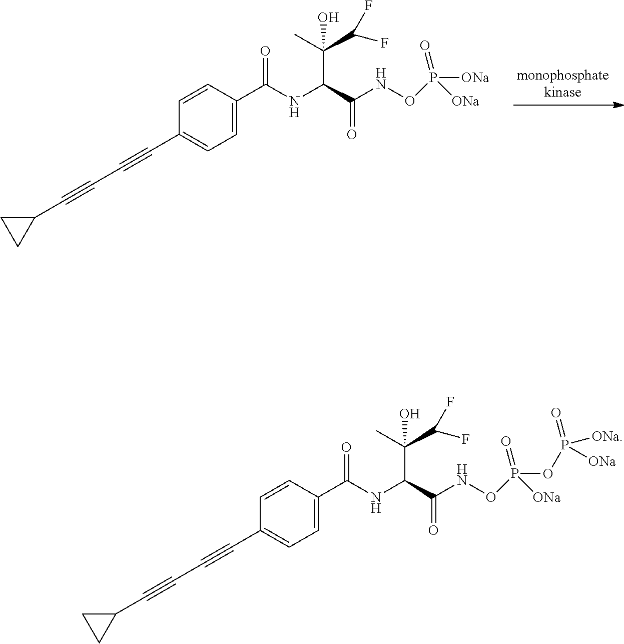

In a second pathway, LPC-233 is treated with a mixture of H₃PO₄ and P₄O₁₀ (P₂O₅). Heat is provided to the reaction mixture. The reaction mixture is then made basic to give the pyrophosphate LPC-233 prodrug. This reaction proceeds according to the following reaction scheme:

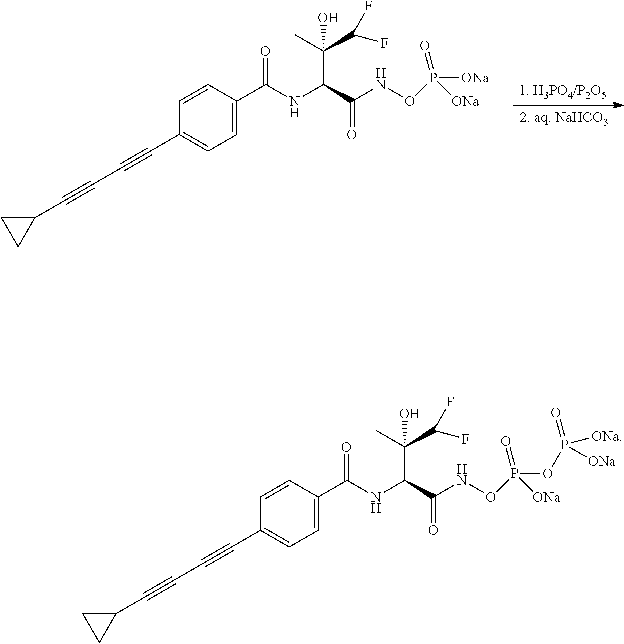

Alternatively, this reaction may proceed by simply adding P₄O₁₀ alone to achieve the pyrophosphate prodrug.

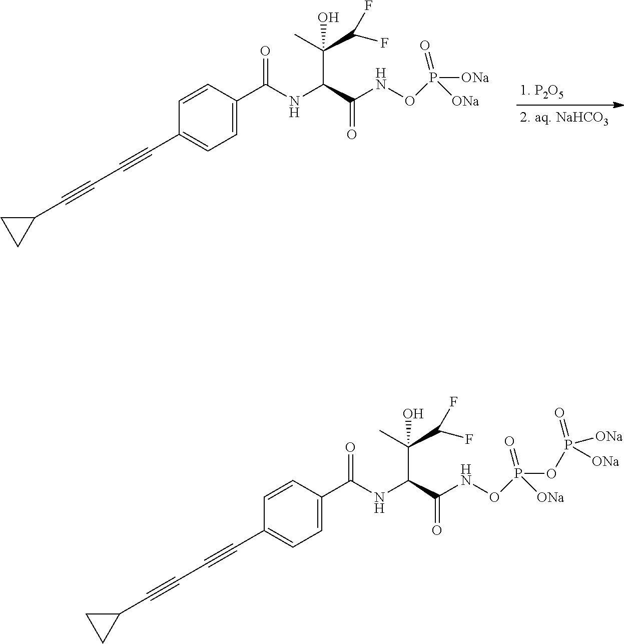

In a third pathway, the reaction proceeds in two steps. First, LPC-233 is reacted with p-toluenesulfonyl chloride (TsCl) and pyridine to transform the hydroxyl group into a leaving group. Then, sodium pyrophosphate is added to give the pyrophosphate prodrug. This reaction proceeds according to the following scheme:

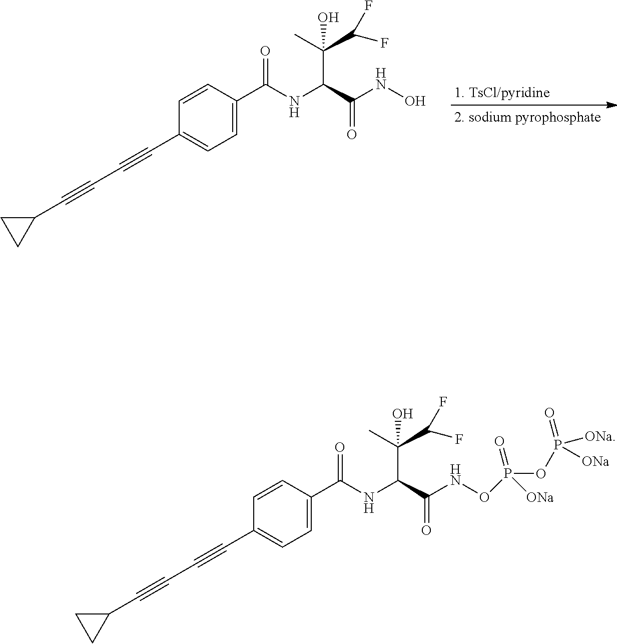

### Example 4: Solubility Study

Maximum solubility of LPC233-phosphate-ProDrug was determined using UV-Vis spectroscopy. First, a sample of LPC-233-Phos-Pro in 5% Captisol was prepared at 1 mg/mL by dissolving 0.7 mg of compound in 700 μL of 5% Captisol and sonicating for 30 minutes. The sample was then serial diluted 2-fold to generate standard solutions ranging from 0.0625-1 mg/mL. The absorption of each sample at 290 nm was determined via nanodrop, and the values were plotted to generate a linear standard curve of y=3.0336x+0.09 with r²=0.998 (v=Abs290 nm, x=[drug]mg/mL). Next, a sample of LPC-233-Phos-Pro was prepared based on an assumed concentration of 300 mg/mL in 5% Captisol and allowed to sonicate for 45 minutes. The sample was then vortexed and centrifuged to pellet any undissolved compound. The supernatant was removed and diluted an initial 30× to a theoretical max concentration of 10 mg/mL. From here, the solution was serial diluted 2-fold up to 256×. The absorption of each dilution at 290 nm was measured via nanodrop and values were plotted to the standard curve. These preparations and measurements were performed in triplicate and averaged. The final maximum solubility of LPC-233-Phos-Pro in 5% Captisol was determined to be ˜260±6 mg/mL.

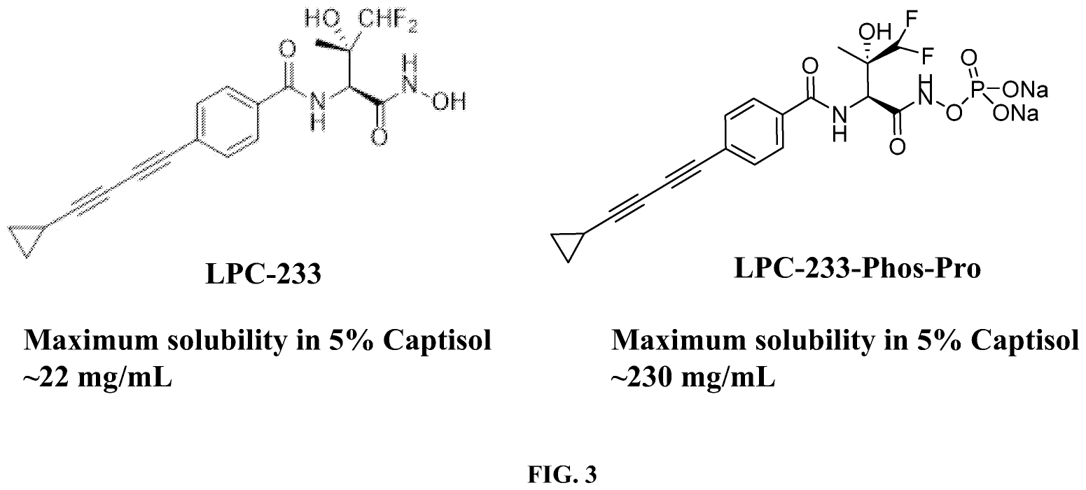

The same procedure listed above was used to determine the maximum solubility of LPC-233 in 5% Captisol with the minor adjustments of targeting 100 mg/mL maximum solubility instead of 300 mg/mL, and initially diluting 50× instead of 30×. These preparations and measurements were performed in triplicate and averaged. The final maximum solubility of LPC-233 in 5% Captisol was determined to be ˜20±2 mg/mL. The obtained data is presented in FIGS. 3A-3B.

### Example 5: Mouse Study

A mice model was created to test the efficacy of LPC-233-Phos-Pro in an Acinetobacter baumannii sepsis model.

Method: Overnight culture of A. baumannii 19606 was inoculated into 50 mL fresh LB with 1:100 dilution. The culture was incubated at 37° C. with vigorous shaking until OD600 reached ˜0.5. The bacteria were harvested, washed, and re-suspended in PBS pH 7.4 buffer. Each CD-1 mouse at the age of 6-7 weeks was injected with about 1.8×109 Acinetobacter baumannii (ATCC 19606) in a volume of 200 μL intraperitoneally. There were ten mice in each group (five male and five female). About 2 hours post infection, 200 μL of vehicle (5% Captisol dissolved in saline), or LPC-233-Phos-Pro in 5% Captisol at specified concentrations were injected intraperitoneally. 12 hours later, 200 μL of vehicle control or LPC-233-Phos-Pro solutions at desired concentrations were administrated via intraperitoneal injections every 12 hours (Q12H) for a total of ten IP injections. The mice were monitored for an additional 10 days and the percentages of survival were recorded. The same procedure was used in a separate experiment to compare the efficacy of LPC-233 and LPC-233-Phos-Pro.

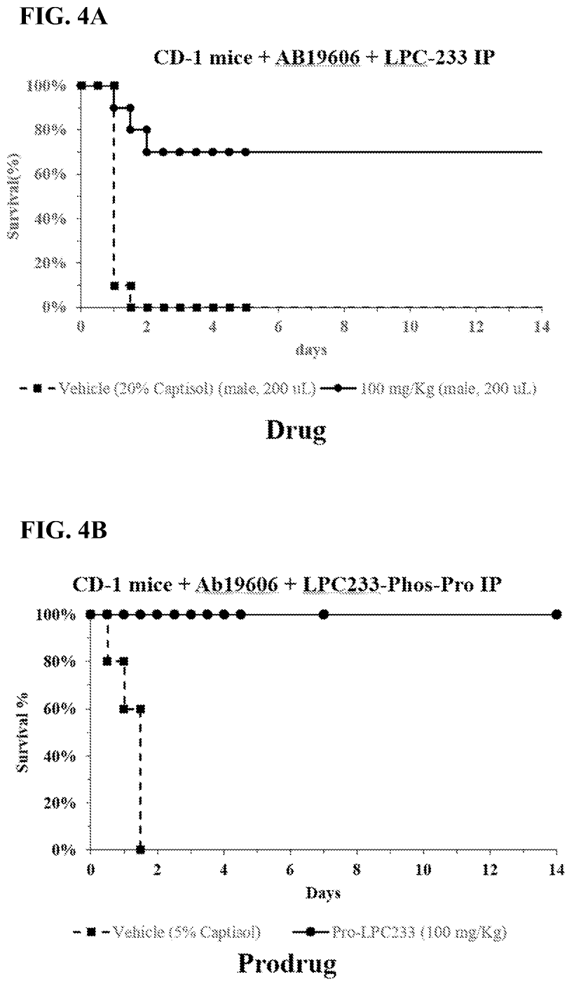

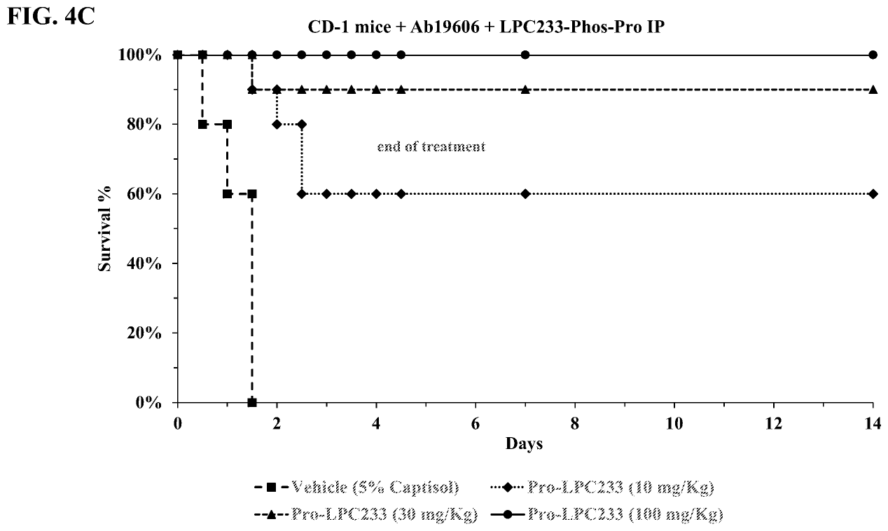

Results: The efficacy of LPC-233-Phos-Pro to rescue mice from lethal infections of Acinetobacter baumannii was compared with that of the parent compound LPC-233, as shown in FIGS. 4A-FIG. 4B. In the LPC-233-Phos-Pro dose response efficacy study, three different dosage of the prodrug, 10 mg/kg, 30 mg/kg and 100 mg/kg were tested in the mice model as shown in FIG. 4C. It was found that all vehicle-treated mice died within 36 hours, whereas the groups treated with LPC-233-Phos-Pro showed dose-dependent survival with a fitted ED50 value of 5.5 mg/kg Q12H. Additionally, 90% of the mice treated with 30 mg/kg Q12H LPC-233-Phos-Pro and all mice treated with 100 mg/kg Q12H of LPC-233-Phos-Pro survived during the treatment and post-treatment observation periods. These results demonstrated that LPC-233-Phos-Pro is highly efficacious against Acinetobacter baumannii 19606 infections in vivo. Additionally, LPC-233-Phos-Pro was proven to be more effective than its parent compound LPC-233 in rescuing mice from lethal A. baumannii 19606 infection, because LPC-233 achieved 70% overall survival and LPC-233-Phos-Pro achieve 100% overall survival at 100 mg/kg Q12H.

Additionally, the Maximum Tolerated 5-day Repeat dose of LPC-233-Phos-Pro was tested in male CD-1 mice (n=3). LPC-233-Phos-Pro was prepared in 5% Captisol and 200 μL of solution was administered via IP injection every 12 hours (Q12H) at doses of 250 mg/kg/Q12H (500 mg/kg/day) over the course of 5 days. The mice were weighed daily to monitor animal survival and weight loss. All mice survived, and no significant weight loss was observed (<10% of initial body weight).

One skilled in the art will readily appreciate that the present disclosure is well adapted to carry out the objects and obtain the ends and advantages mentioned, as well as those inherent therein. The present disclosure described herein are presently representative of preferred embodiments, are exemplary, and are not intended as limitations on the scope of the present disclosure. Changes therein and other uses will occur to those skilled in the art which are encompassed within the spirit of the present disclosure as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

What is claimed is:

1. A compound of Formula (I): 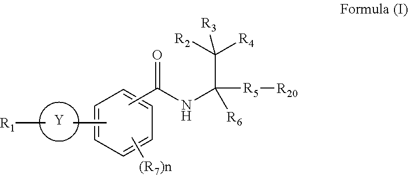
or a stereo-isomer, an enantiomer, a polymorph, a pharmaceutically acceptable salt, an ester, or a derivative thereof,
wherein
Y represents aryl optionally substituted with R₈, heteroaryl optionally substituted with R₈, cycloalkyl, or heterocyclyl optionally substituted with R₈;
n is an integer selected from 0, 1, 2, 3, or 4;
R₁ is hydrogen, C₁-C₆ alkyl optionally substituted with R₉, aryl optionally substituted with R₉, heteroaryl optionally substituted with R₉, or heterocyclyl optionally substituted with R₉;
R₂ is C₁-C₆ alkyl, C₁-C₆haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₃ is C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₄ is C₁-C₆ haloalkyl;
R₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₆ is hydrogen or C₁-C₆ alkyl;
each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups that when attached to the same carbon atom form ═O;
each R₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups that when attached to the same carbon atom form ═O; and
each R₂₀ is independently selected from Formula (IIa), Formula (IIb), or Formula (IIc): 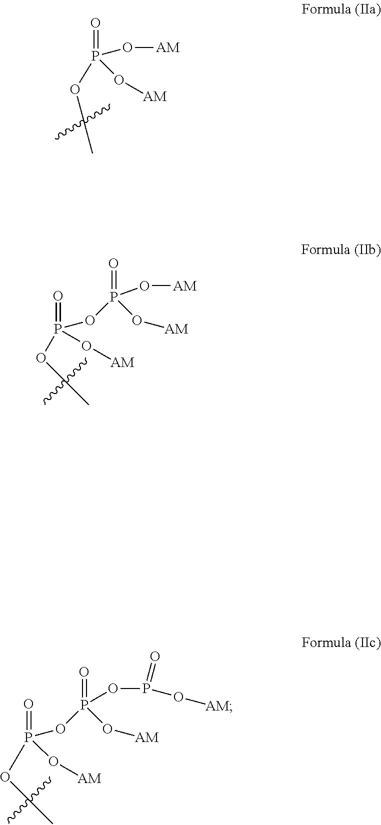
wherein each AM is independently selected from Na, Li, K, Rb, or C₁-C₆ alkyl.

2. The compound of claim 1, wherein:
R₂ is C₁-C₆ alkyl, hydroxyl, or —O—R₂₀;
R₃ is C₁-C₆ alkyl, hydroxyl, or —O—R₂₀;
and
R₅ is selected from the group consisting of —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, and —CONH—NH—.

3. The compound of claim 1 or claim 2, wherein the moiety of Formula (I) 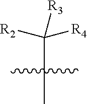
has a structure of Formula (IIIa) or Formula (IIIb):

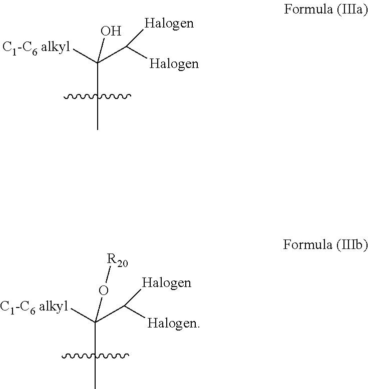

4. The compound of any one of claims 1-3, wherein Y is selected from the group consisting of phenyl, pyridinyl, pyrimidinyl, ethyleniminyl, cyclopropyl, pyrrolyl, furanyl, tetrahydrofuranyl, aziridinyl, pyrrolidinyl, pyrrolidonyl, thiophenyl, piperidinyl, thiazol, azulenyl, naphthyl, and imidazole, which are optionally substituted with R₈.

5. A compound of Formula (IV): 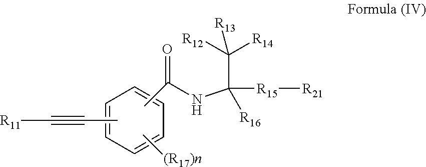
or a stereo-isomer, an enantiomer, a polymorph, a pharmaceutically acceptable salt, an ester or a derivative thereof, wherein
n is an integer selected from 0, 1, 2, 3, or 4;
R₁₁ is —C≡C—R₃₃, C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈;
wherein R₃₃ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₉, (C₃-C₈ cycloalkyl) C₁-C₆ alkyl- optionally substituted with R₁₉, a C₃-C₇ cyclyl or heterocyclyl optionally substituted with R₁₉;
R₁₂ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₃ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₄ is C₁-C₆ haloalkyl;
R₁₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₁₆ is hydrogen or C₁-C₆ alkyl;
each R₁₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₁₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —C₁-C₆ alkyl-OH, —C₁-C₆ alkyl-(C₁-C₆ alkoxy), —C₁-C₆ alkyl-NH₂, —C₁-C₆alkyl-NH—C₁-C₆ alkyl), —C₁-C₆ alkyl-N(C₁-C₆ alkyl)₂, —C₁-C₆ alkyl-NH(SO₂ C₁-C₆ alkyl), —CONH₂, —CONH(C₁-C₆ alkyl), —CON(C₁-C₆ alkyl)₂, —NH(SO₂ C₁-C₆ alkyl), —CONH—OH, —CONH—OCO(C₁-C₆ alkyl), —C(NH)NH—OH, —CONH—NH₂, —CO(C₁-C₆ alkyl), —CO₂H, —CO₂(C₁-C₆ alkyl), —C₁-C₆ alkyl-CONH₂, —C₁-C₆ alkyl-CONH(C₁-C₆ alkyl), —C₁-C₆ alkyl- CON(C₁-C₆ alkyl)₂, —C₁-C₆ alkyl-CONH—OH, —C₁-C₆ alkyl-CO(C₁-C₆ alkyl), —C₁-C₆ alkyl- CO₂H, and —C₁-C₆ alkyl-CO₂(C₁-C₆ alkyl); or two R₁₈ groups when attached to the same carbon atom form ═O;
each R₁₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —C₁-C₆ alkyl-OH, —C₁-C₆ alkyl-(C₁-C₆ alkoxy), —C₁-C₆ alkyl-NH₂, —C₁-C₆ alkyl-NH—C₁-C₆ alkyl), —C₁-C₆ alkyl-N(C₁-C₆ alkyl)₂, —C₁-C₆ alkyl-NH(SO₂ C₁-C₆ alkyl), —CONH₂, —CON(C₁-C₆ alkyl), —CON(C₁-C₆ alkyl)₂, —NH(SO₂ C₁-C₆ alkyl), —CONH—OH, —CONH—OCO(C₁-C₆ alkyl), —C(NH)NH—OH, —CONH—NH₂, —CO(C₁-C₆ alkyl), —CO₂H, —CO₂(C₁-C₆ alkyl), —C₁-C₆ alkyl-CONH₂, —C₁-C₆ alkyl-CON(C₁-C₆ alkyl), —C₁-C₆ alkyl-CON(C₁-C₆ alkyl)₂, —C₁-C₆ alkyl-CONH—OH, —C₁-C₆ alkyl-CO(C₁-C₆ alkyl), —C₁-C₆ alkyl-CO₂H, and —C₁-C₆ alkyl-CO₂(C₁-C₆ alkyl); or two R₁₉ groups when attached to the same carbon atom form ═O; and
each R₂₁ is independently selected from Formula (IIa), Formula (IIb), or Formula (IIc): 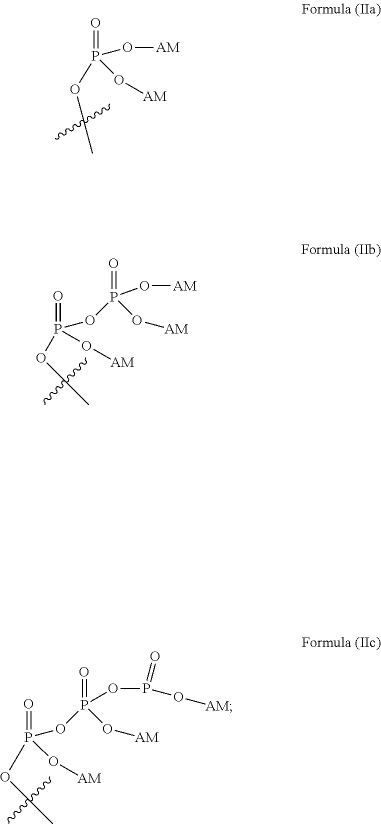
wherein each AM is independently selected from Na, Li, K, Rb, or C₁-C₆ alkyl.

6. The compound of claim 5, wherein
R₁₂ is C₁-C₆ alkyl, hydroxyl, or —O—R₂₁;
R₁₃ is C₁-C₆ alkyl, hydroxyl, or —O—R₂₁;
and
R₁₅ is selected from the group consisting of —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, and —CONH—NH—.

7. The compound of claim 5 or claim 6, wherein the moiety of Formula (IV) 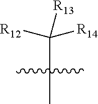
is of Formula (Va) or Formula (Vb):

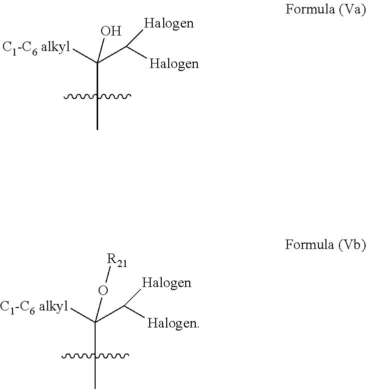

8. The compound of any one of claims 5-7, wherein
R₁₁ is —C≡C—R₃₃, or C₃-C₈ cycloalkyl optionally substituted with R₁₈, (C₃-C₈ cycloalkyl)-C₁-C₆ alkyl- optionally substituted with R₁₈, or heterocyclyl optionally substituted with R₁₈; and
R₃₃ is —C₁-C₆ alkyl-CO₂H, C₃-C₈ cycloalkyl optionally substituted with R₁₉, (C₃-C₈ cycloalkyl) C₁-C₆ alkyl- optionally substituted with R₁₉, C₃-C₇ cyclyl, or heterocyclyl optionally substituted with R₁₉.

9. A compound of Formula (VI): 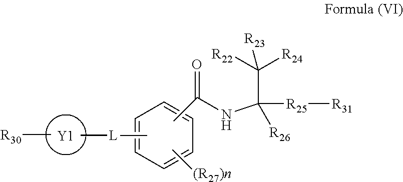
or a stereo-isomer, an enantiomer, a polymorph, a pharmaceutically acceptable salt, an ester, or a derivative thereof, wherein
L represents a linker, wherein L is absent or is selected from the group consisting of —C≡C—C≡C—, or —C≡C—R₃₂, wherein R₃₂ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₂₉, (C₃-C₈ cycloalkyl)-C₁-C₆ alkyl- optionally substituted with R₂₉, C₃-C₇ cyclyl, and heterocyclyl optionally substituted with R₂₉;
Y1 represents aryl optionally substituted with R₂₈, heteroaryl optionally substituted with R₂₈, cycloalkyl, or heterocyclyl optionally substituted with R₂₈;
n is an integer selected from 0, 1, 2, 3, or 4;
R₃₀ is hydrogen, C₁-C₆ alkyl optionally substituted with R₂₉, aryl optionally substituted with R₂₉, heteroaryl optionally substituted with R₂₉, or heterocyclyl optionally substituted with R₂₉;
R₂₂ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), -hydroxy(C₁-C₆ alkyl), -alkoxy(C₁-C₆ alkyl), or -amino(C₁-C₆ alkyl);
R₂₃ is halogen, —C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₄ is C₁-C₆ haloalkyl;
R₂₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₂₆ is hydrogen or C₁-C₆ alkyl;
each R₂₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
each R₃₁ is independently selected from Formula (IIa), Formula (IIb), or Formula (IIc): 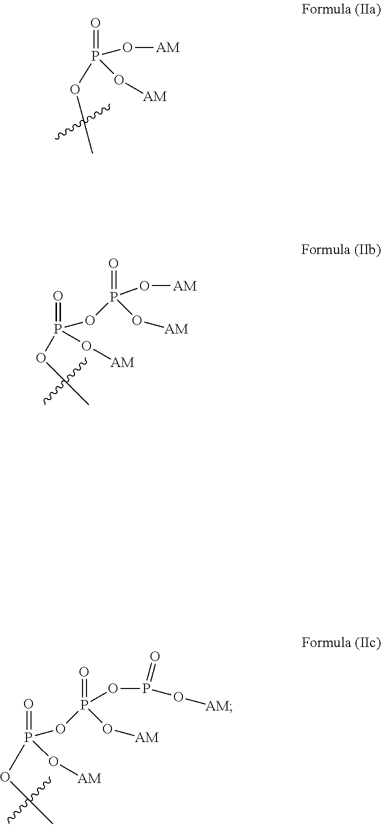
wherein each AM is independently selected from Na, Li, K, Rb, or a C₁-C₆ alkyl.

10. The compound of claim 9, wherein
R₂₂ is C₁-C₆ alkyl, hydroxyl, or —O—R₃₁;
R₂₃ is C₁-C₆ alkyl, hydroxyl, or —O—R₃₁;
and
R₂₅ is selected from the group consisting of —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, and —CONH—NH—.

11. The compound of claim 9 or claim 10, wherein the moiety of Formula (VI) 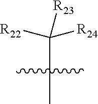
is of Formula (VIIa) or Formula (VIIb):

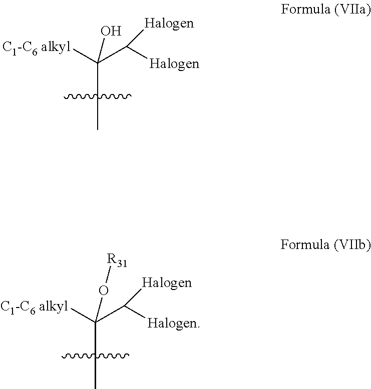

12. The compound of any one of claims 9-11, wherein Y₁ is selected from the group consisting of phenyl, pyridinyl, pyrimidinyl, ethyleniminyl, cyclopropyl, pyrrolyl, furanyl, tetrahydrofuranyl, aziridinyl, pyrrolidinyl, pyrrolidonyl, thiophenyl, piperidinyl, thiazol, azulenyl, naphthyl, and imidazole, which are optionally substituted with R₂₈.

13. The compound of any one of claims 1-12, wherein AM is Na or methyl.

14. The compound according to any one of claims 1-13, having one of the following formulas: 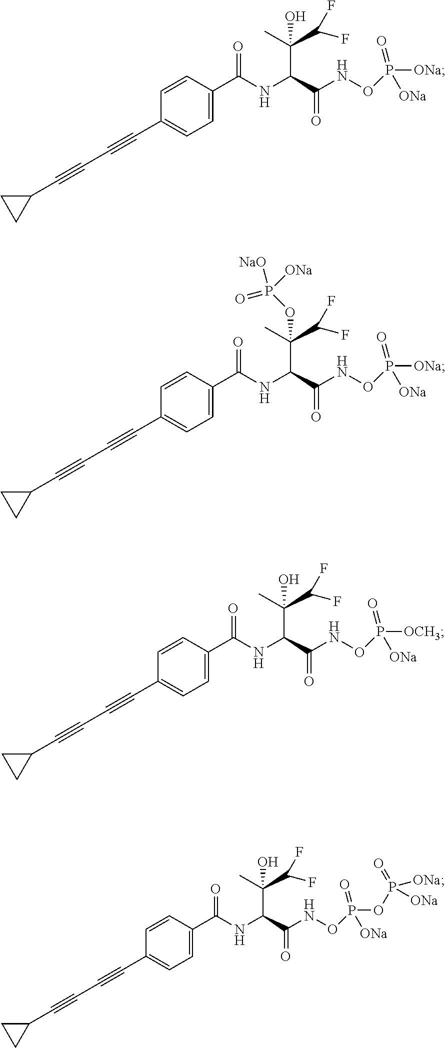
or a polymorph, a pharmaceutically acceptable salt, an ester or a derivative thereof.

15. A compound according to any one of claims 1-13, having one of the following formulas: 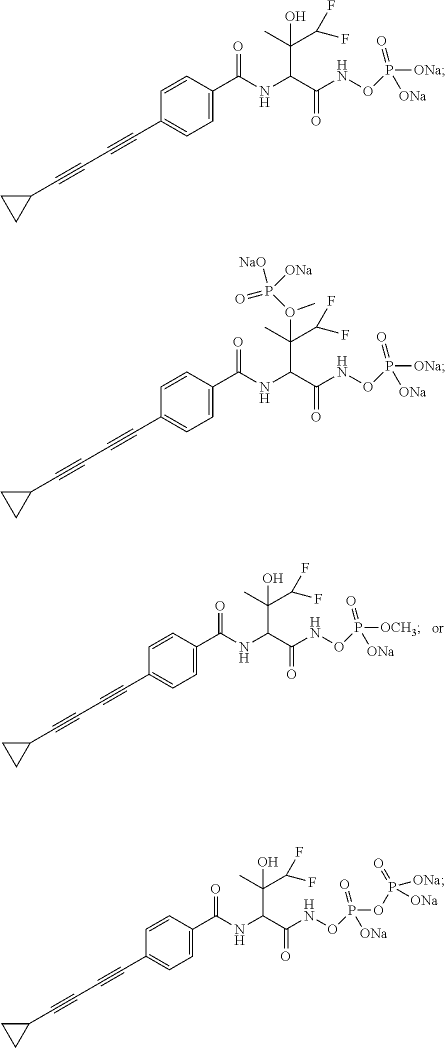
or a stereo-isomer an enantiomer, a polymorph, a pharmaceutically acceptable salt, an ester or a derivative thereof.

16. A compound of any one of claims 1-13 having one of the following formulas: 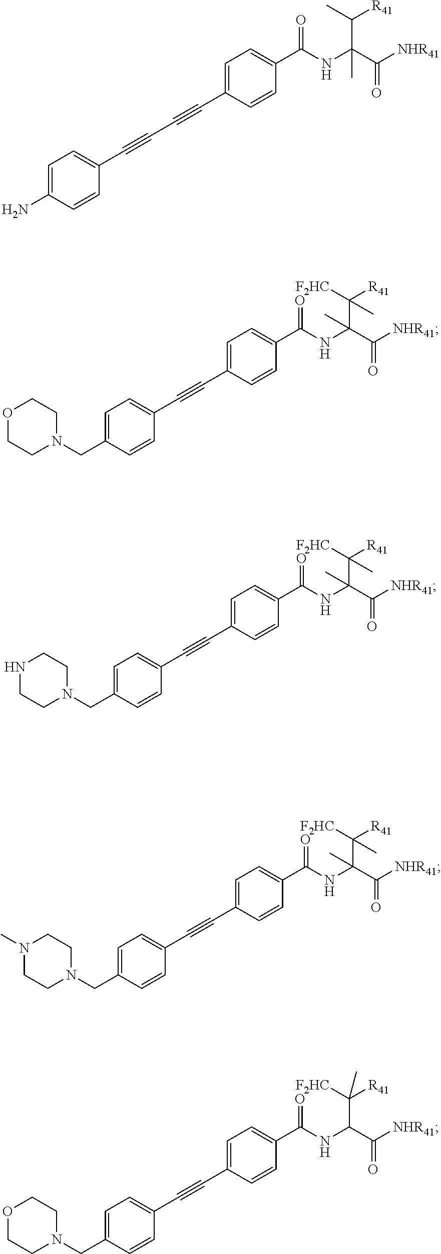 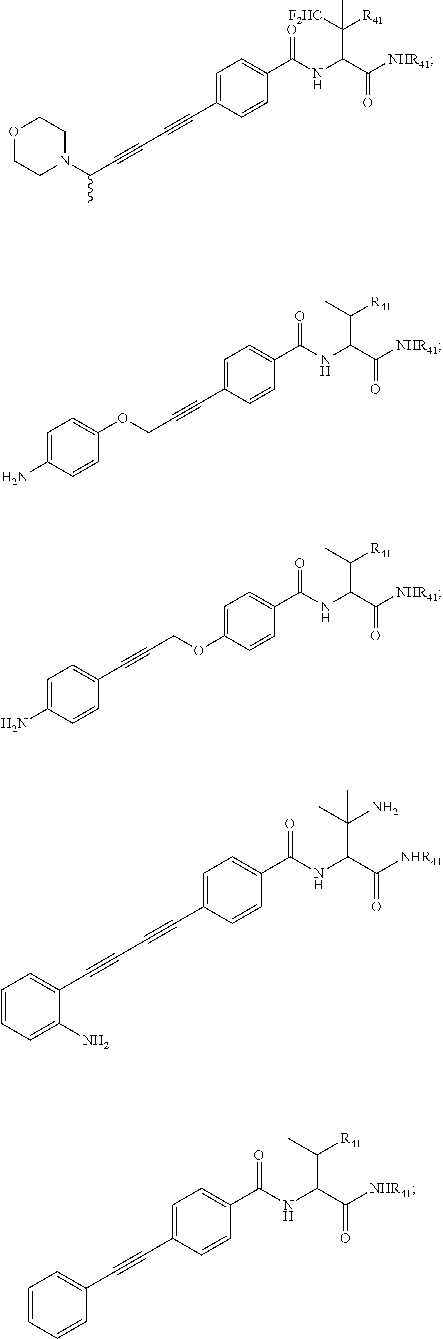 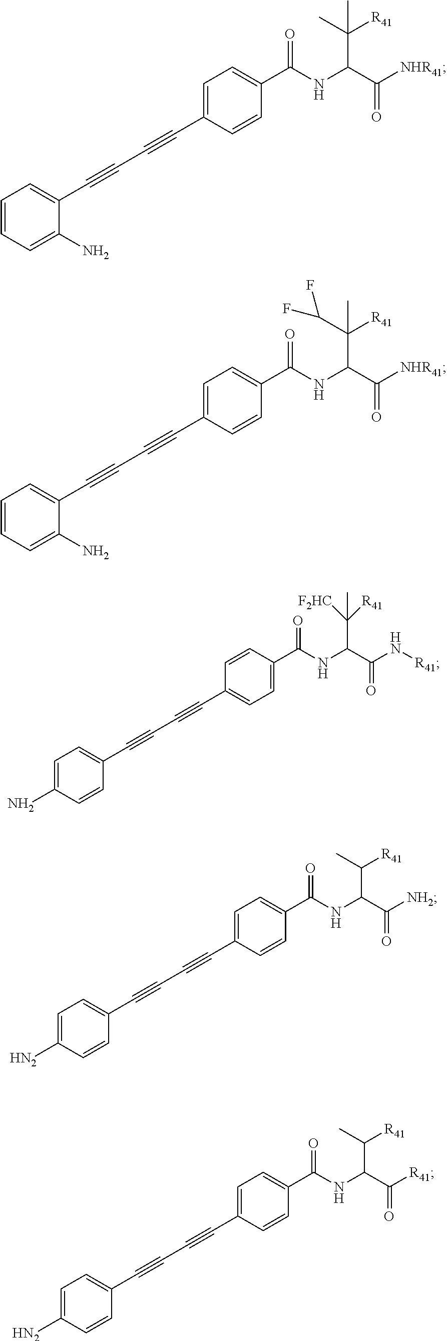 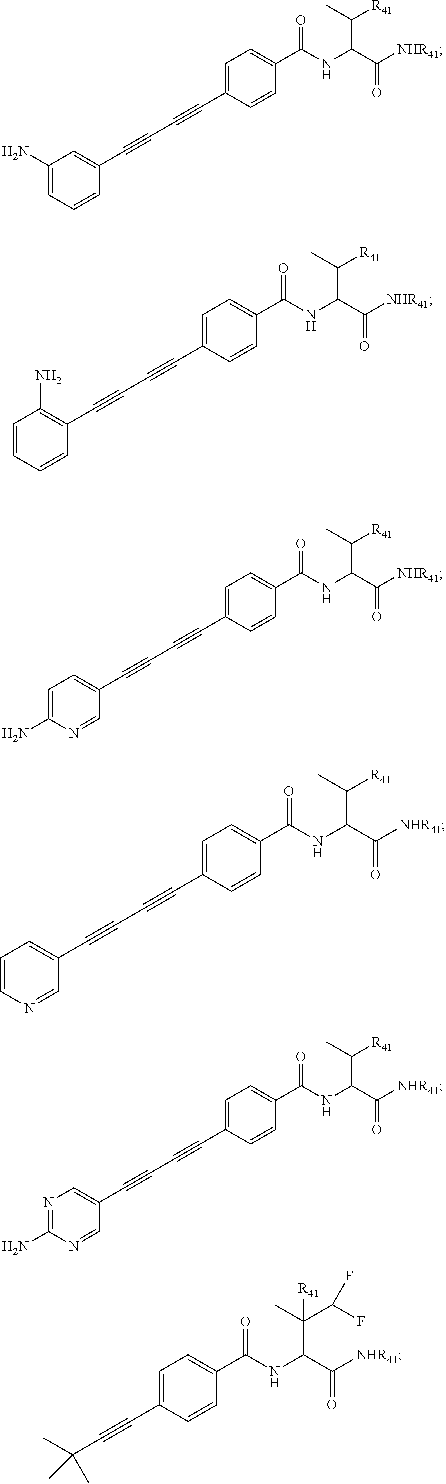 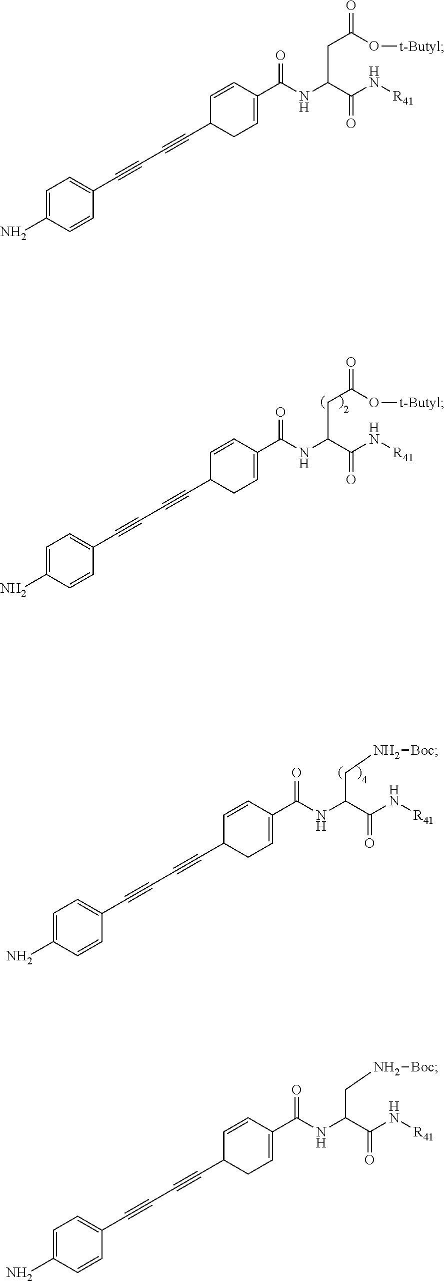 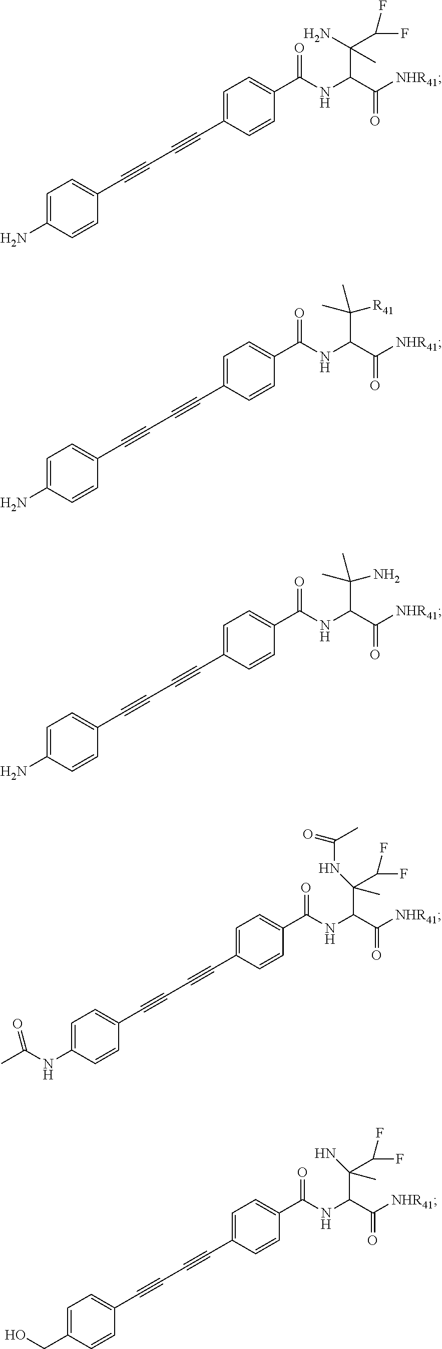 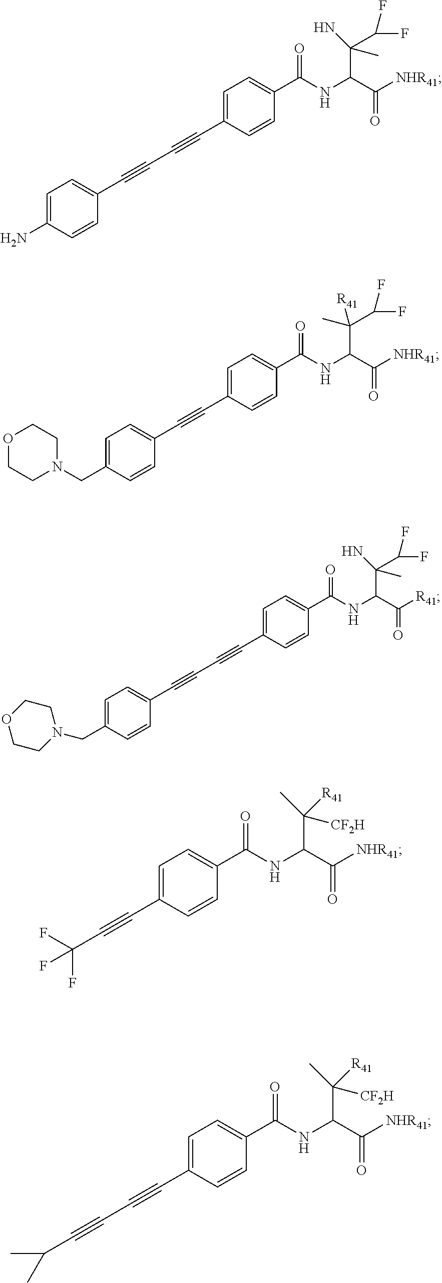 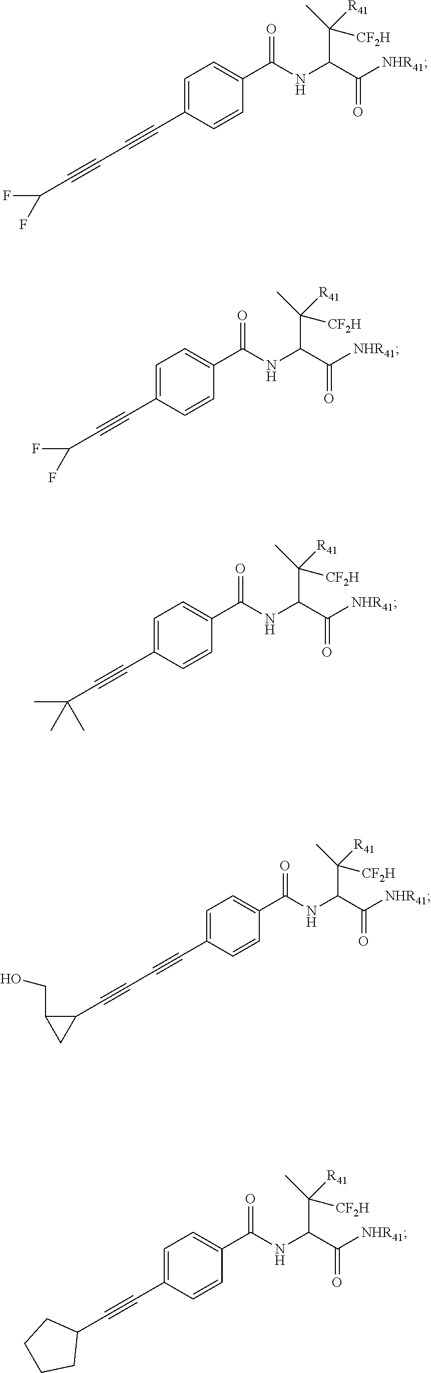 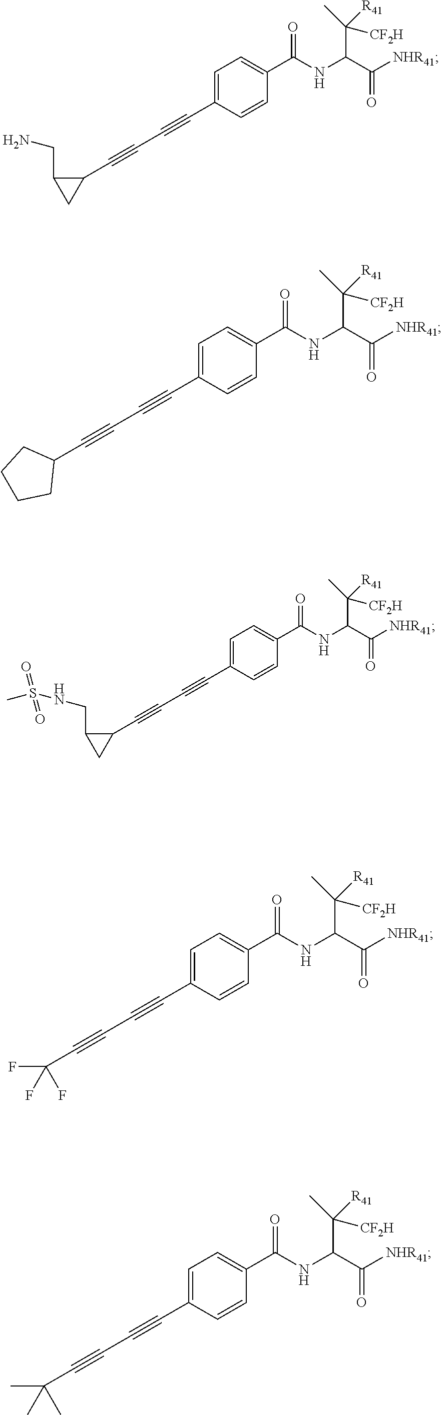 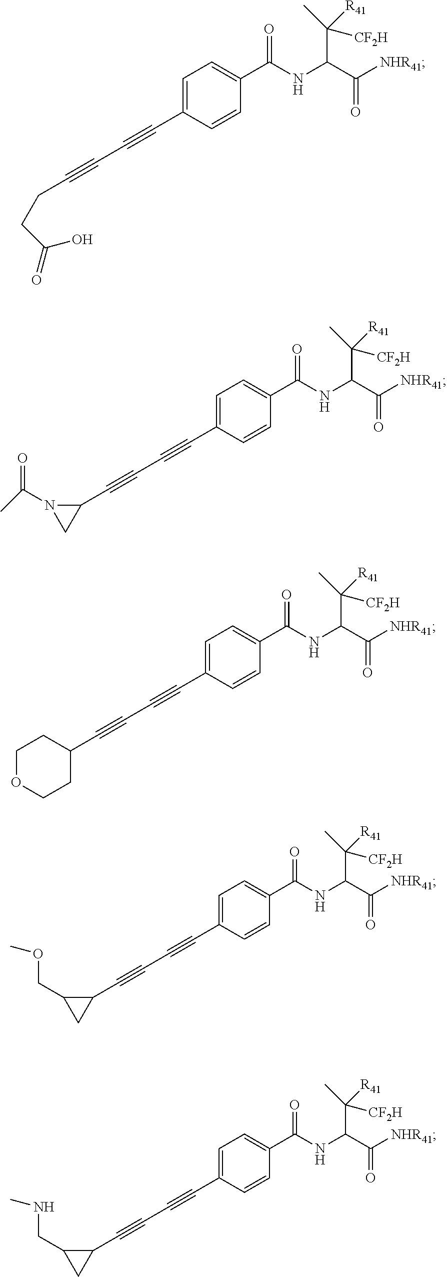 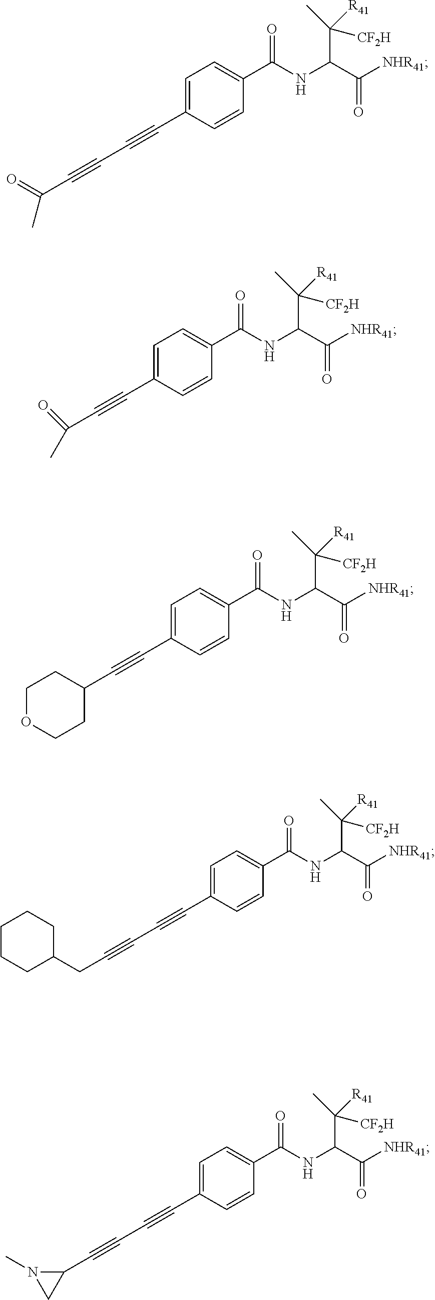 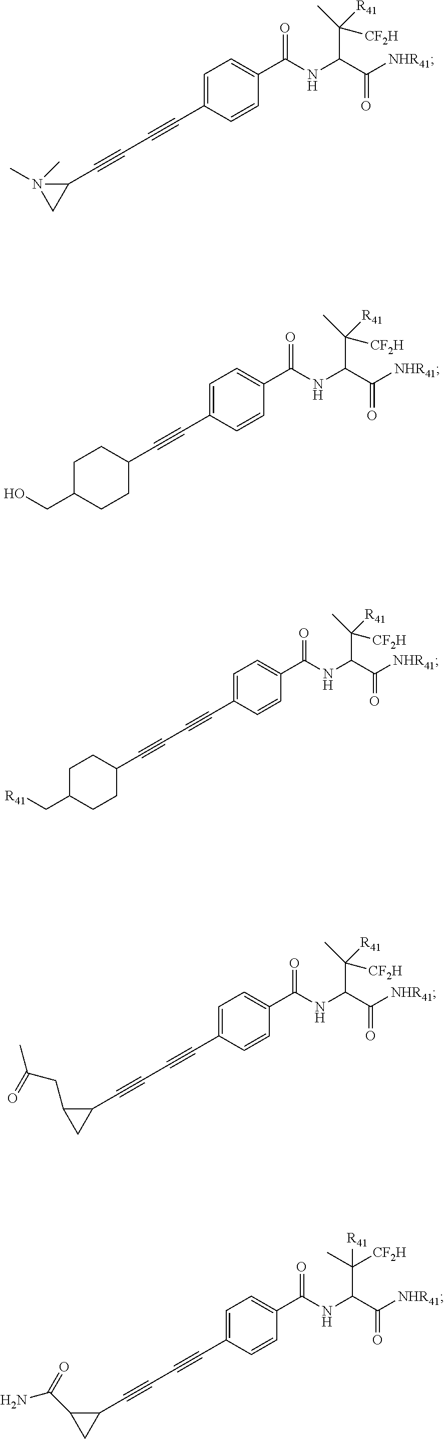 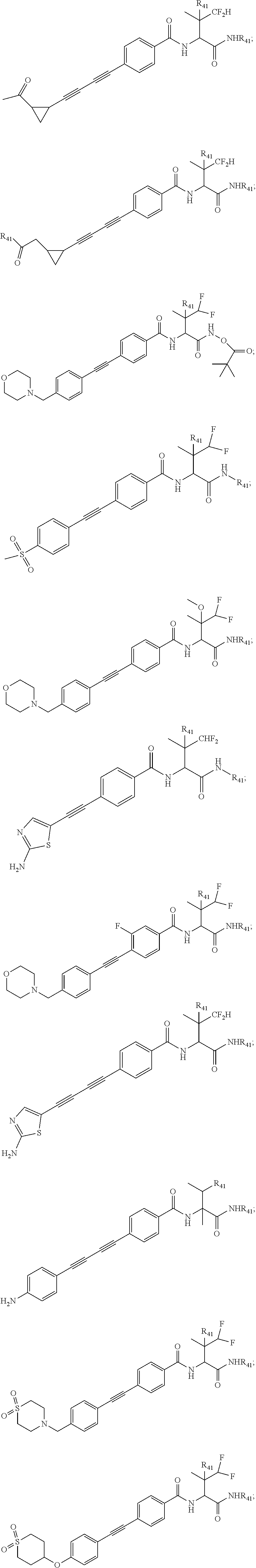 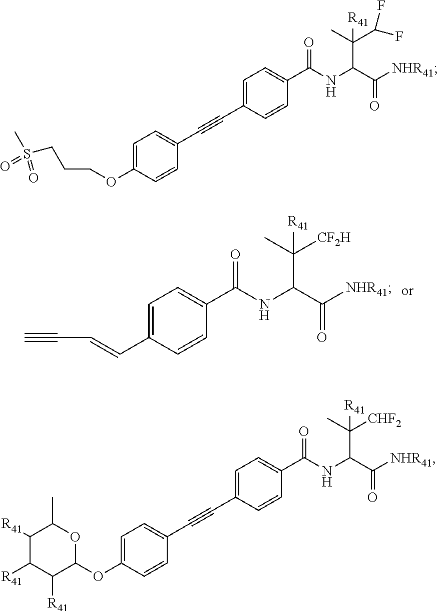
wherein each R₄₁ is independently selected from —OH, Formula (IIa), Formula (IIb), or Formula (IIc), 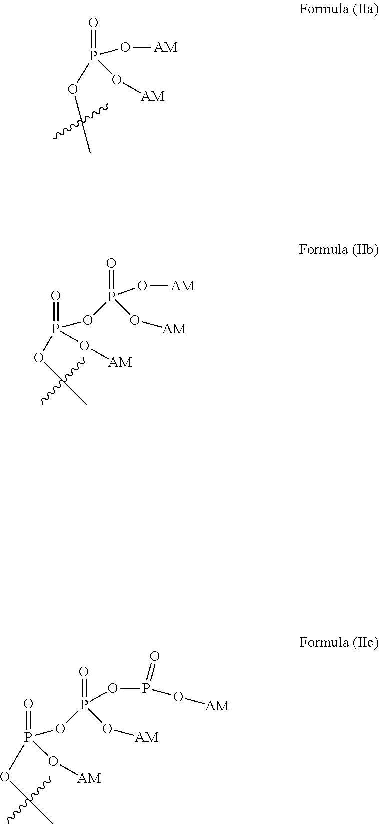
and wherein each AM is independently selected from Na, Li, K, Rb, or a C₁-C₆ alkyl.

17. A mono-phosphate, di-phosphate or tri-phosphate prodrug of a compound selected from the following formulas, wherein the phosphate is formed with a hydroxyl group or a hydroxamate oxo group of the compound: 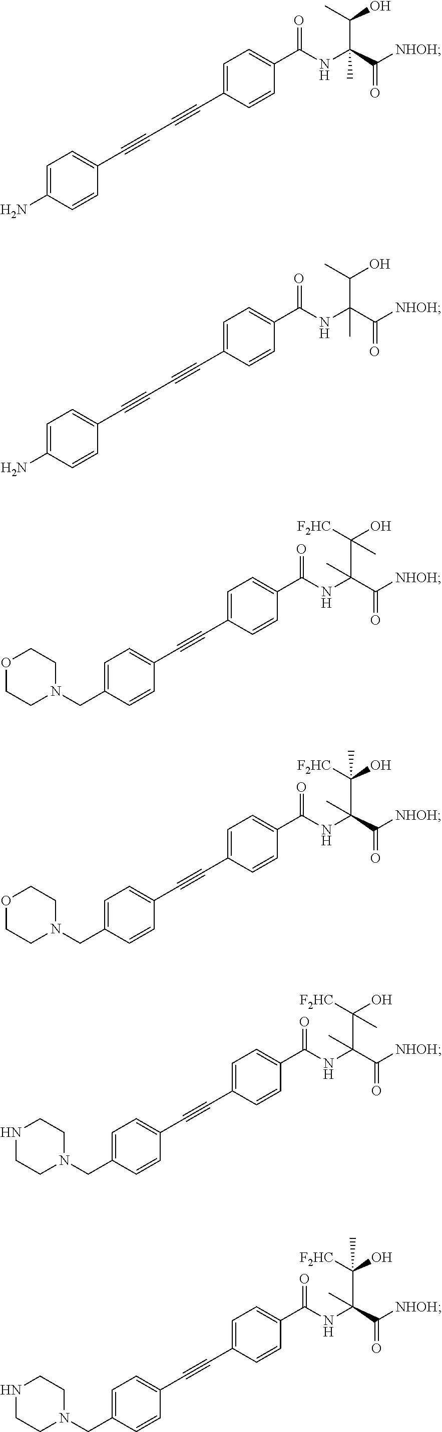 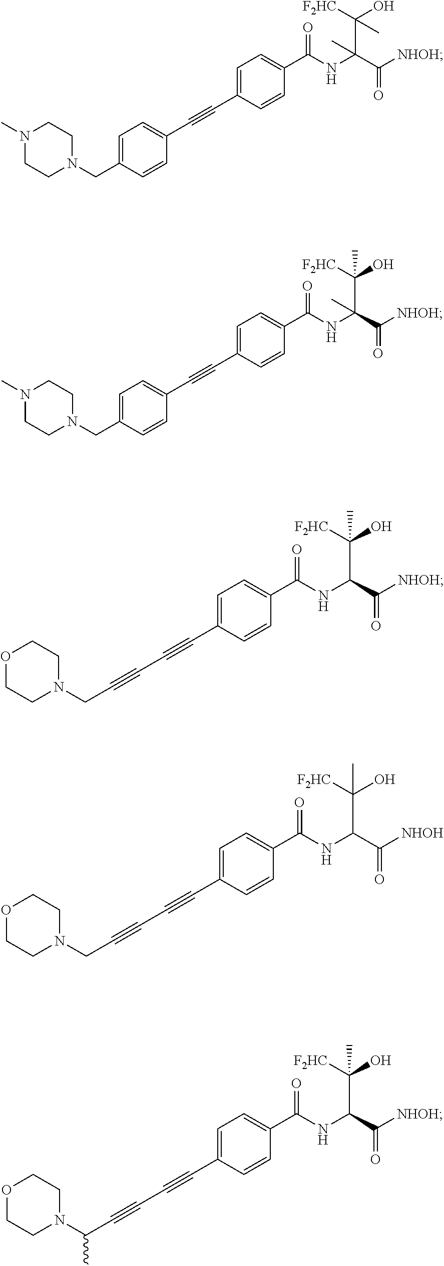 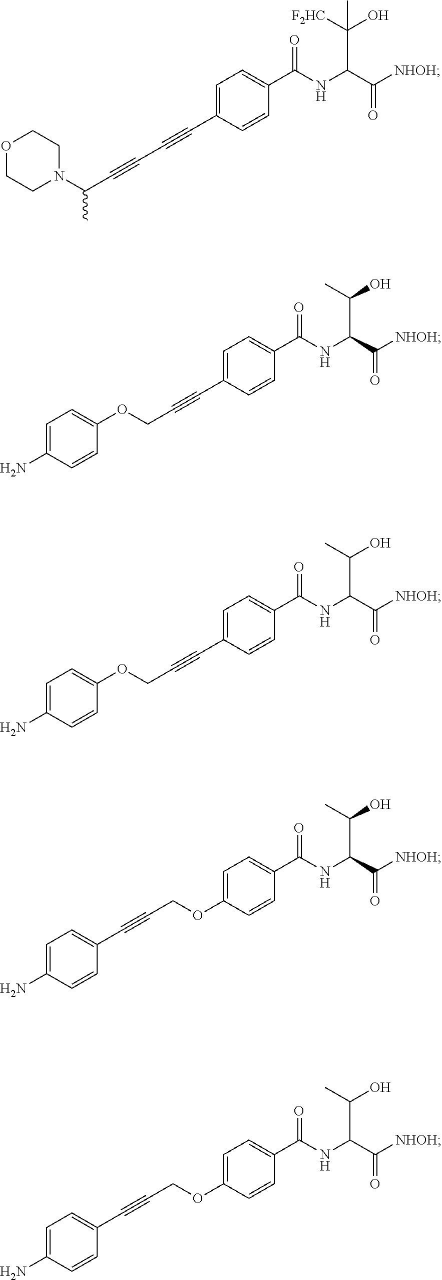 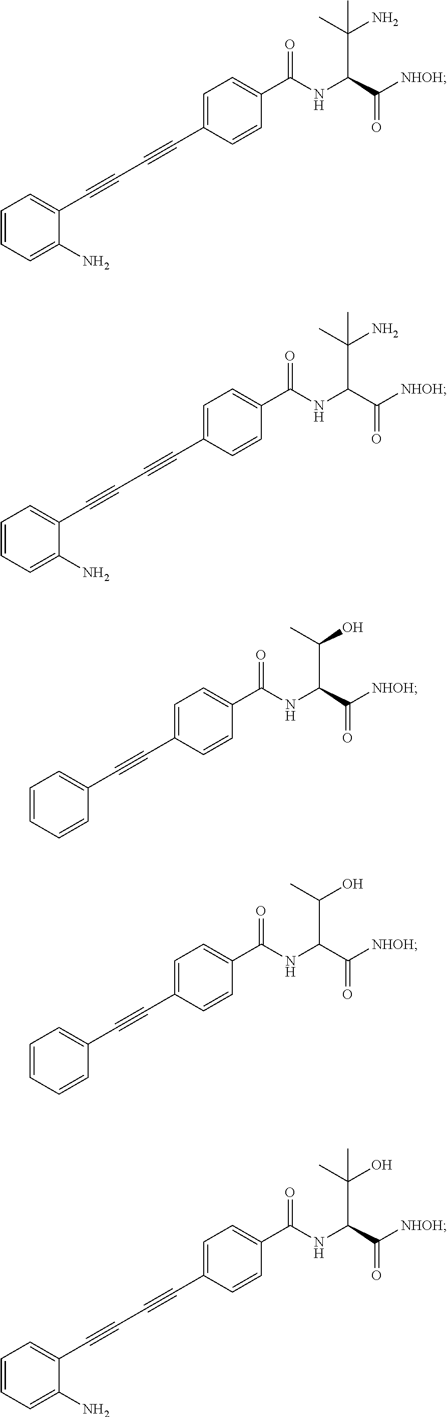 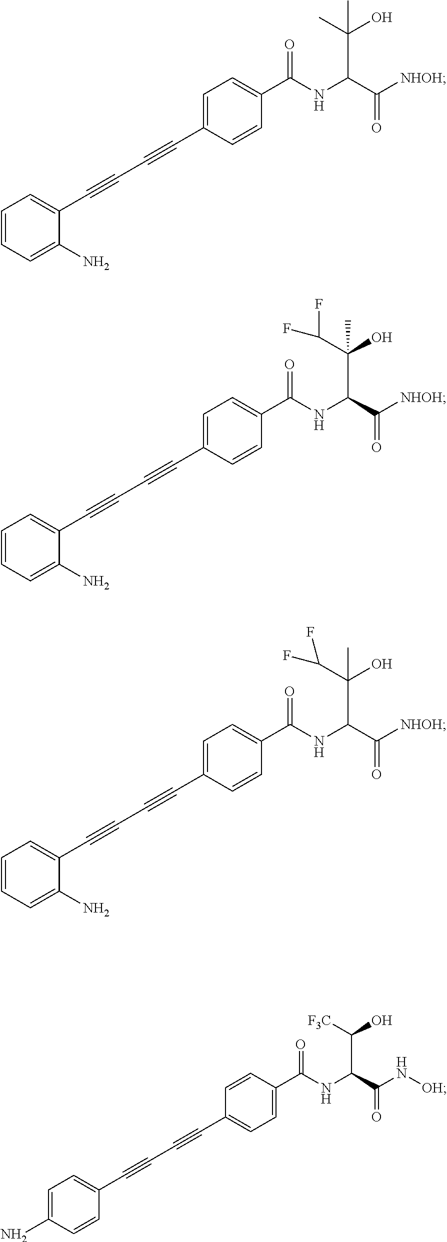 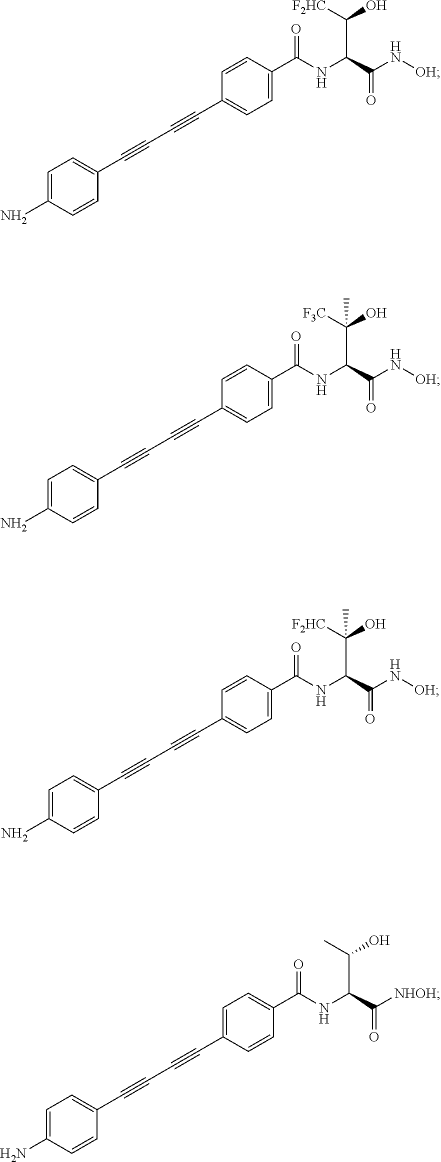 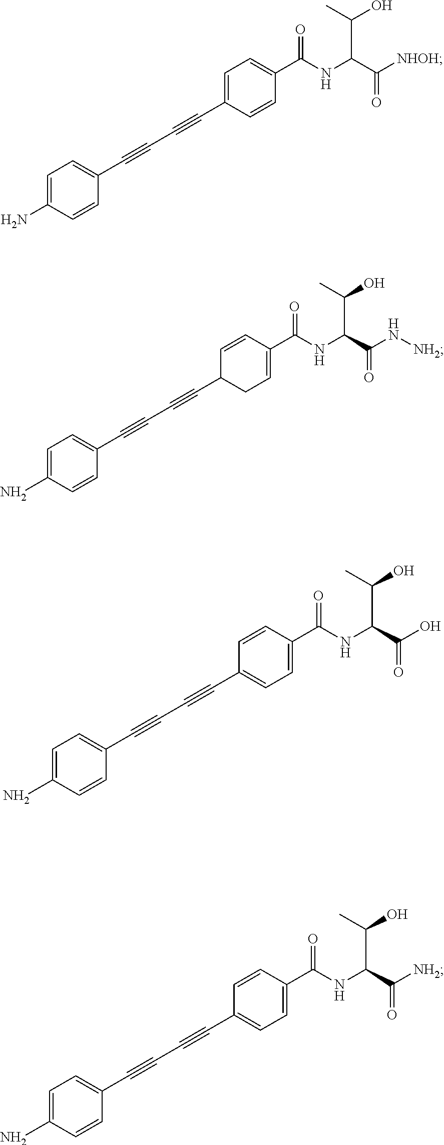 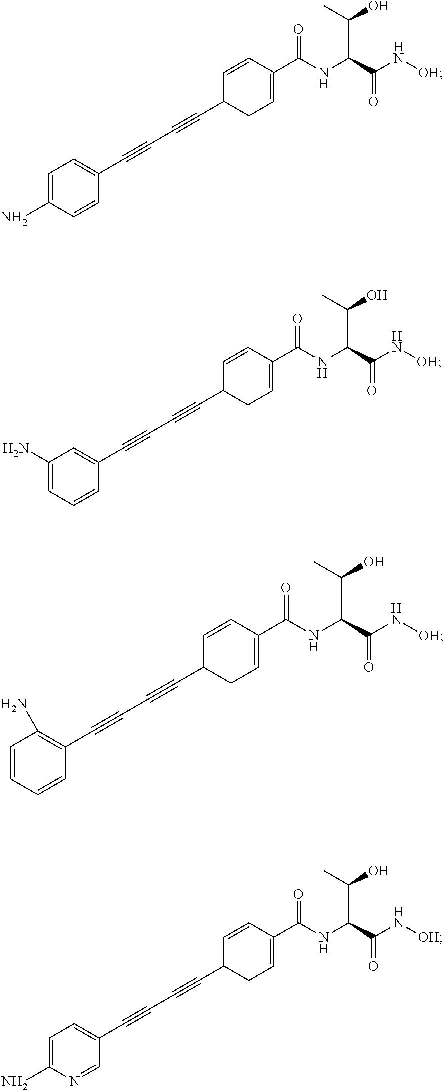 [SMILES: C[C@@H](O)[C@H](NC(=O)C1=CCC(C#CC#Cc2ccc(N)cc2)C=C1)C(=O)NO ; C[C@@H](O)[C@H](NC(=O)C1=CCC(C#CC#Cc2cccc(N)c2)C=C1)C(=O)NO ; C[C@@H](O)[C@H](NC(=O)C1=CCC(C#CC#Cc2ccccc2N)C=C1)C(=O)NO ; C[C@@H](O)[C@H](NC(=O)C1=CCC(C#CC#Cc2ccc(N)nc2)C=C1)C(=O)NO] 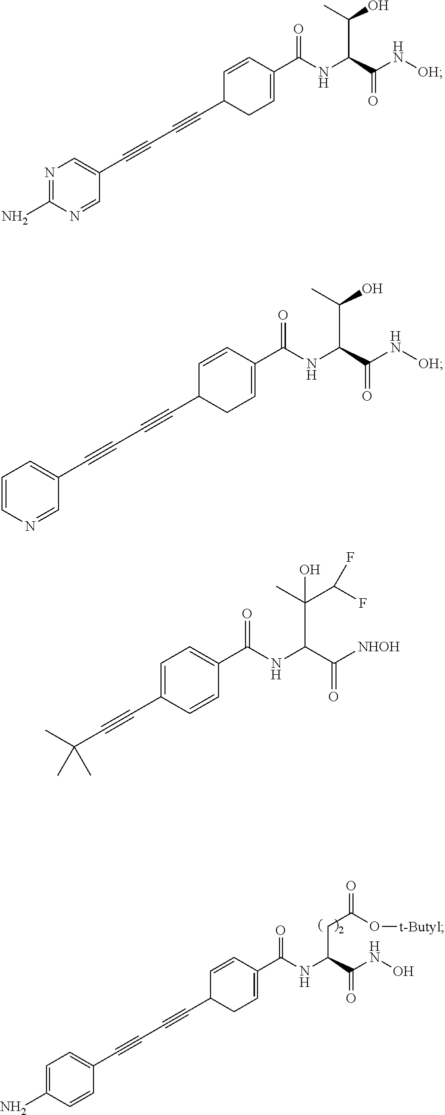 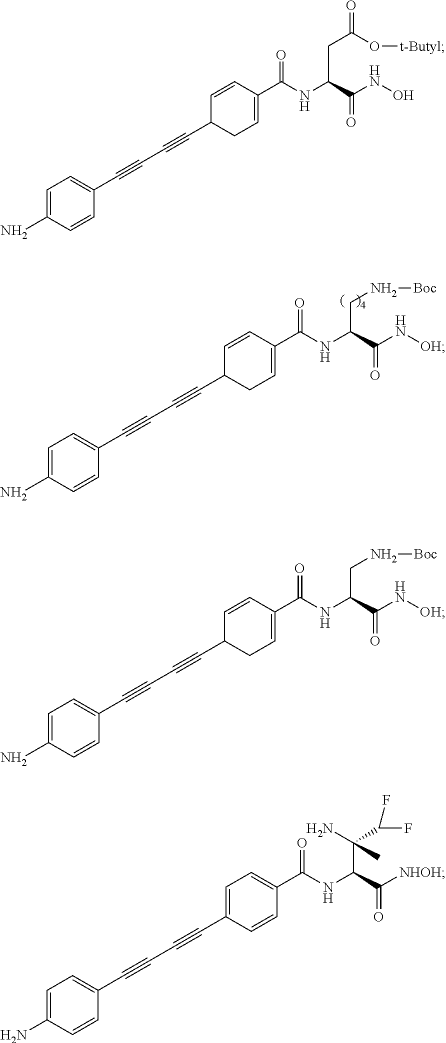 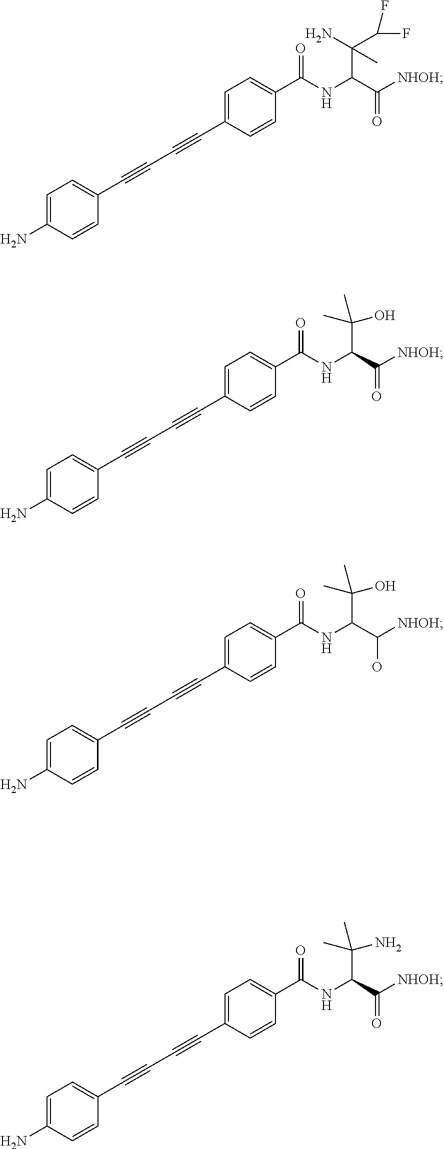 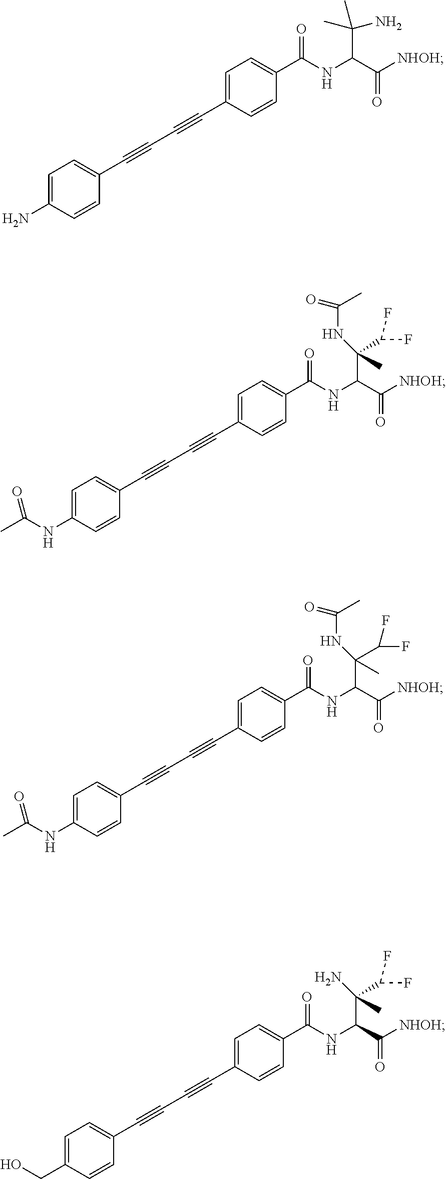 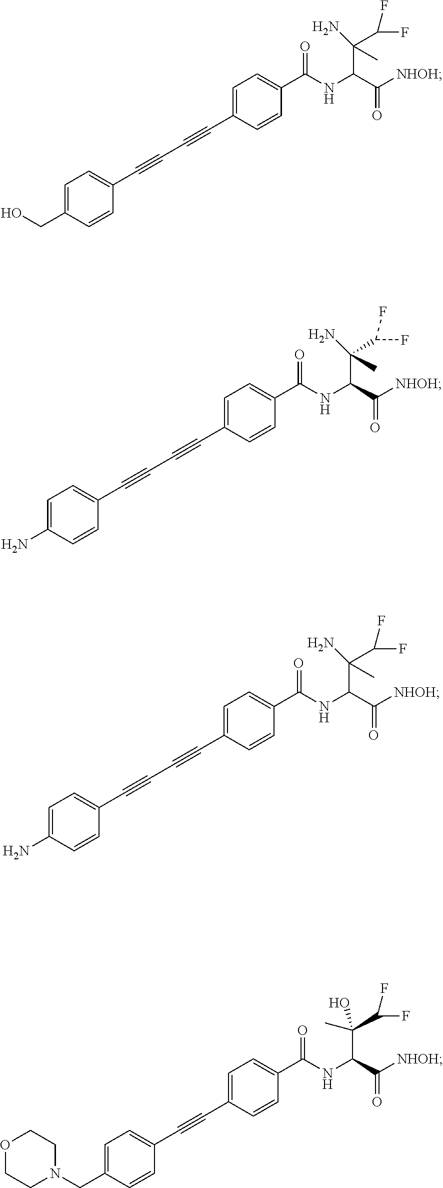 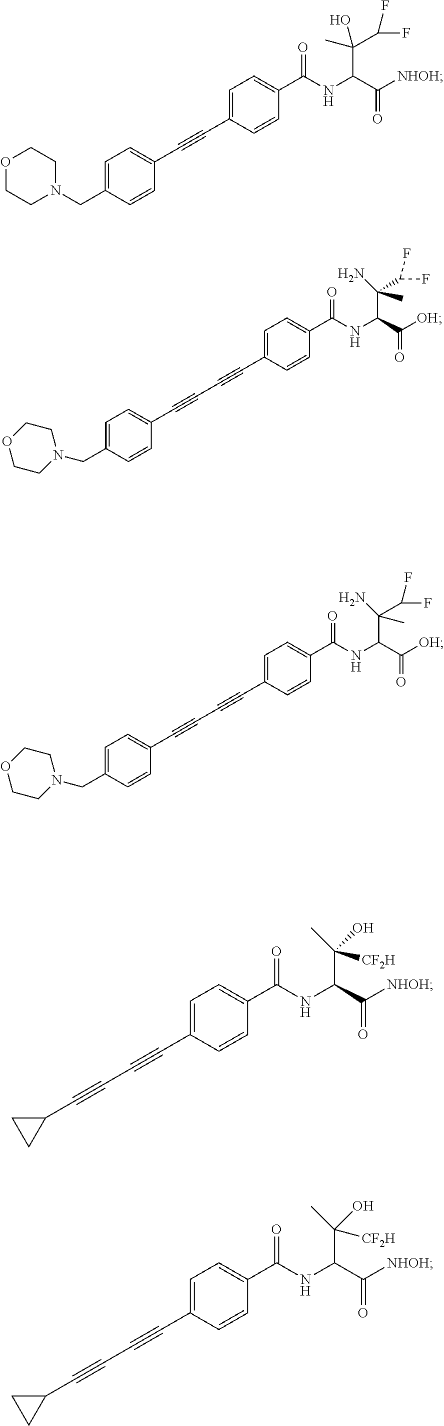 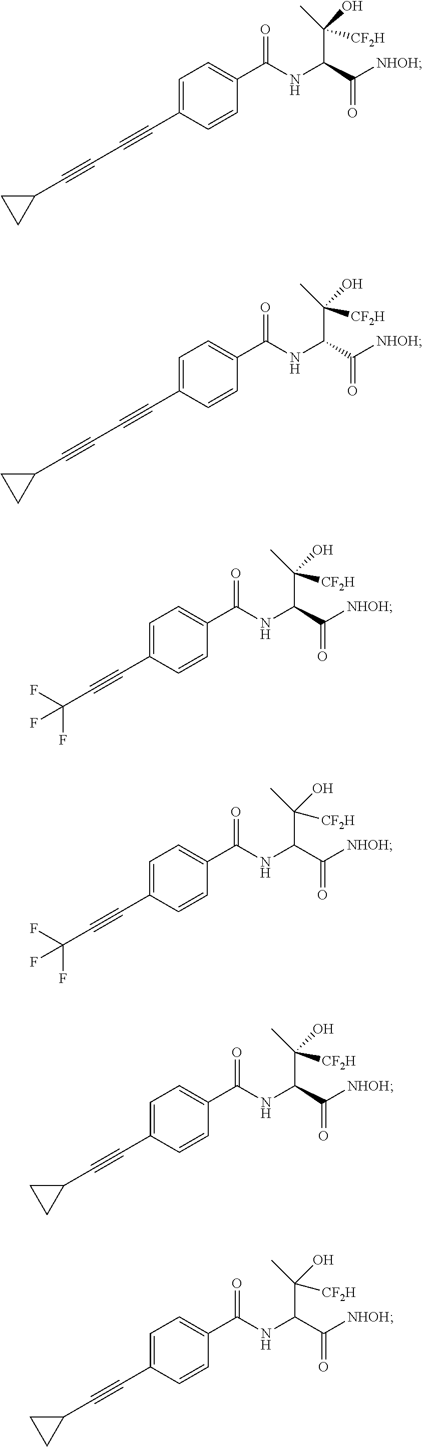 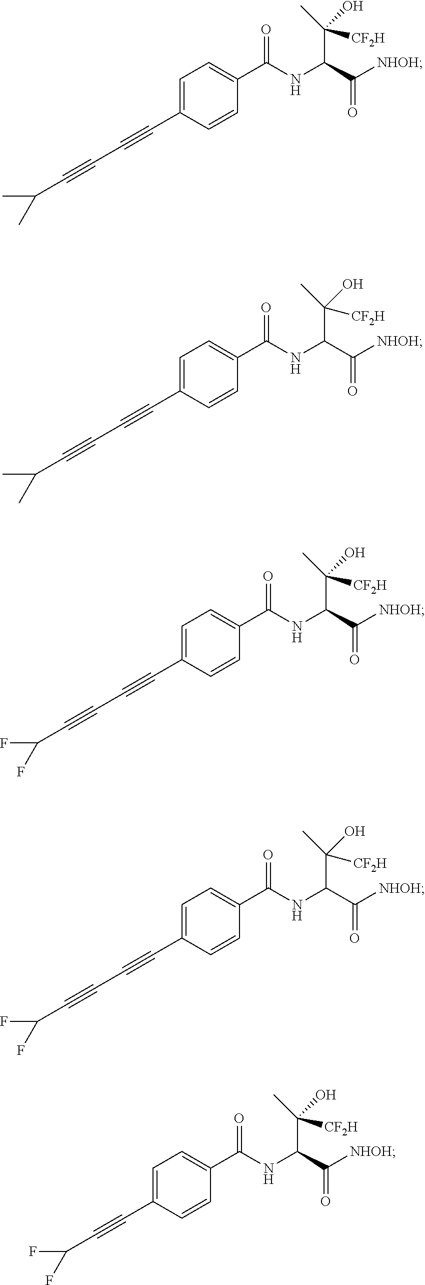 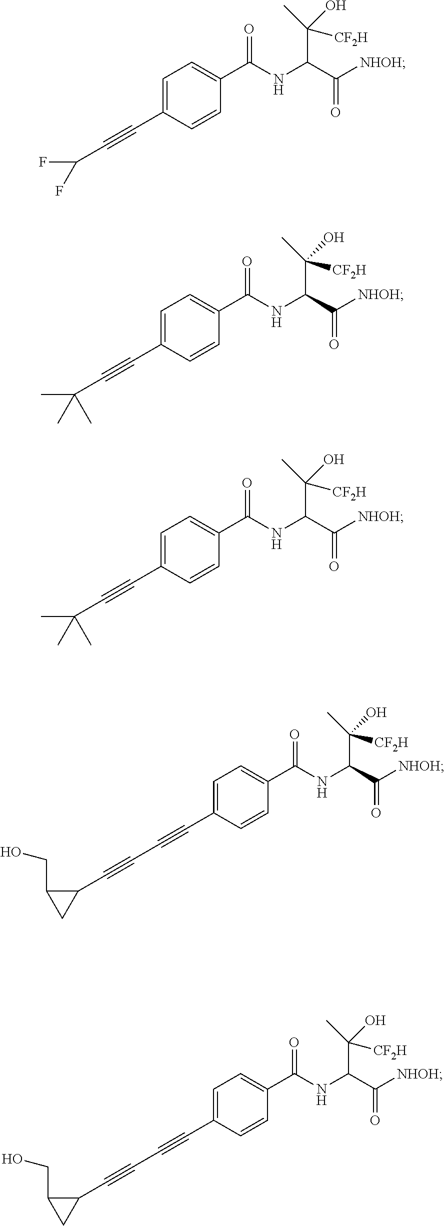 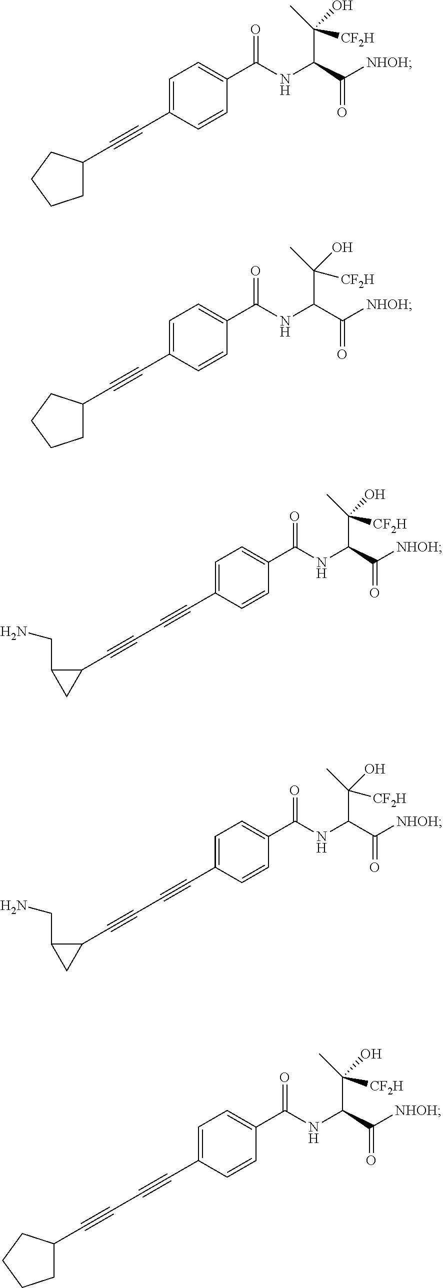 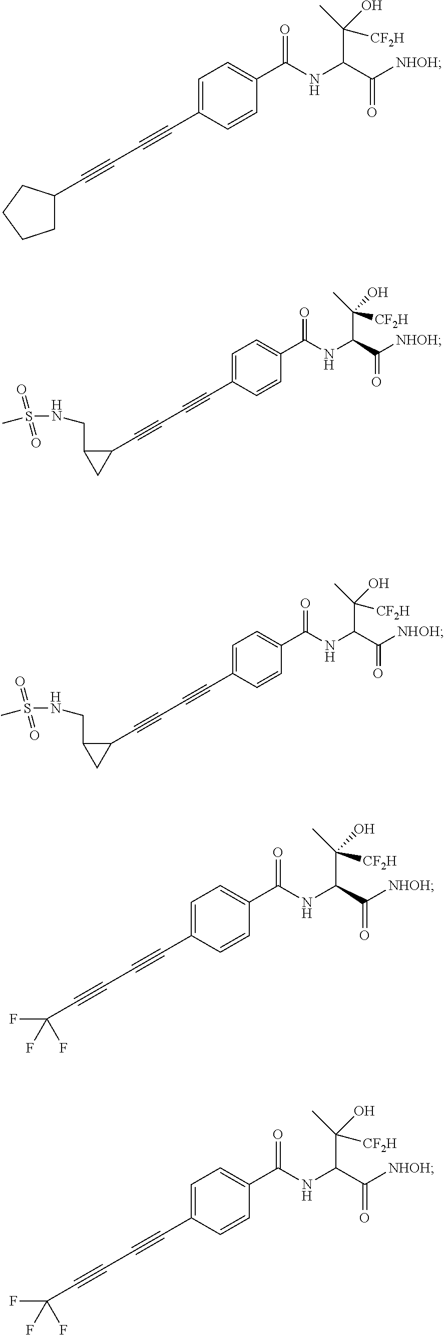 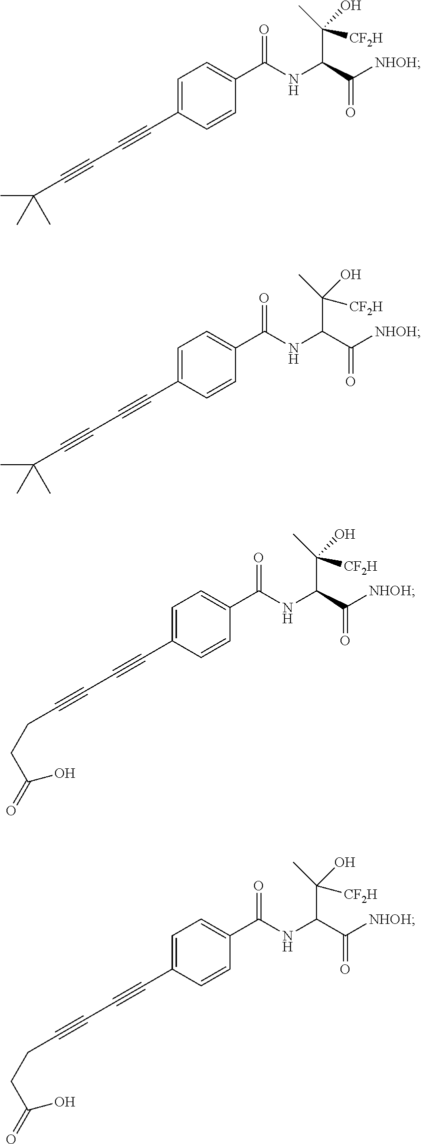 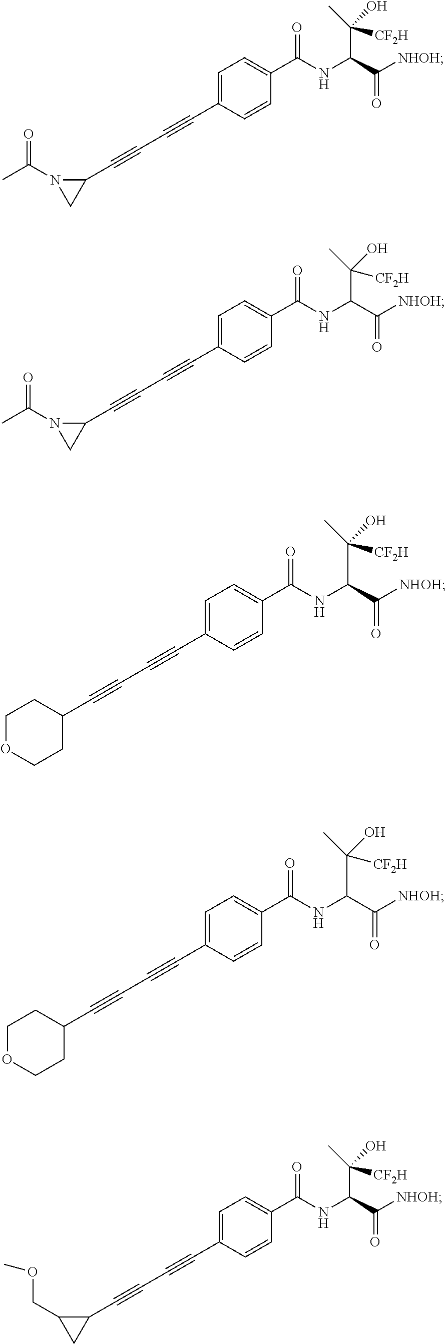 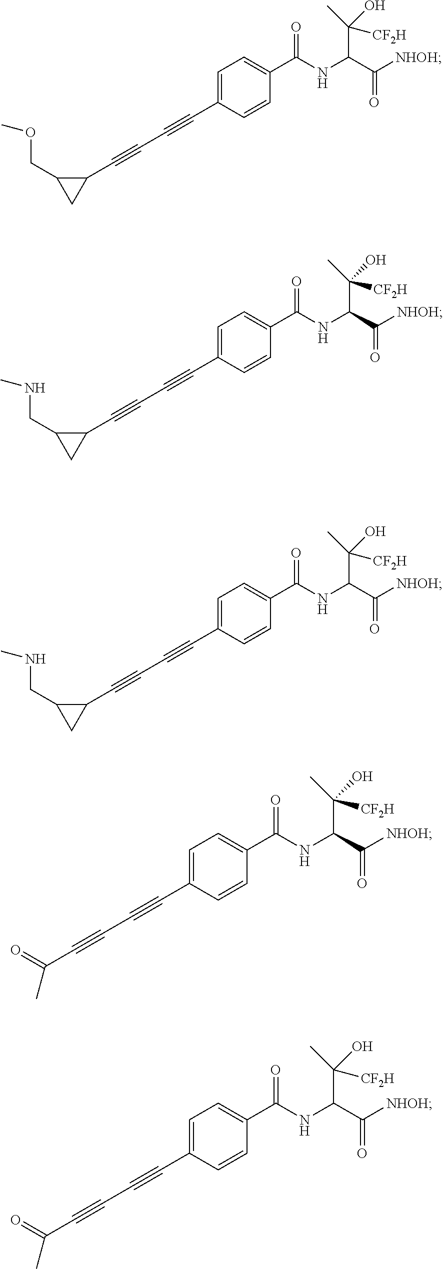 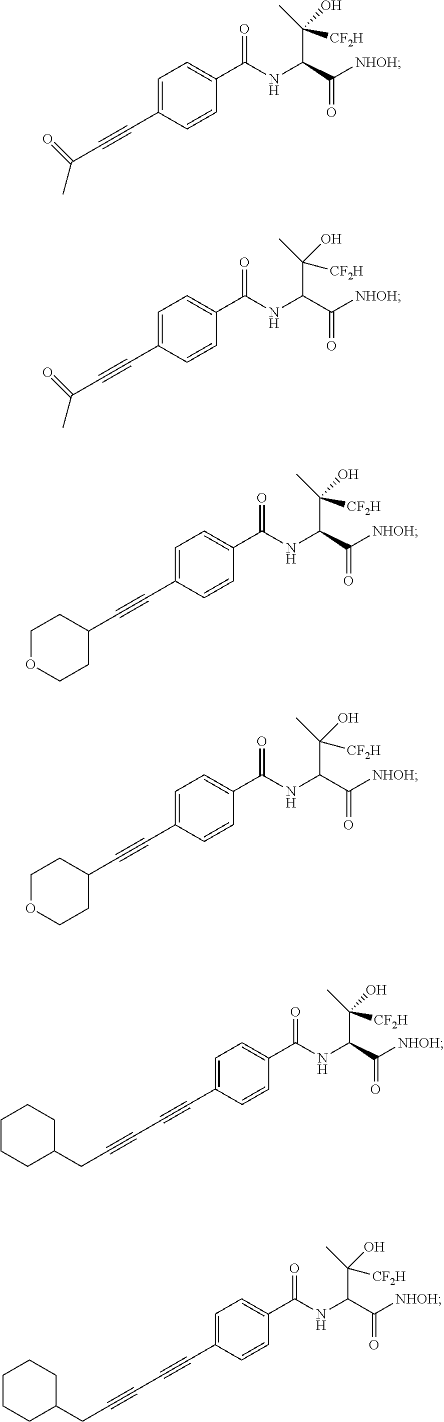 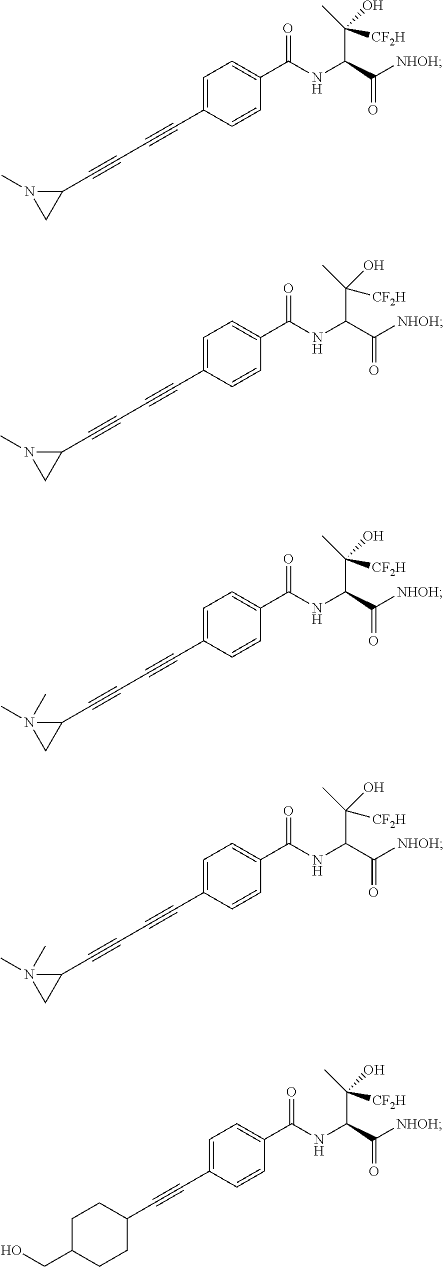 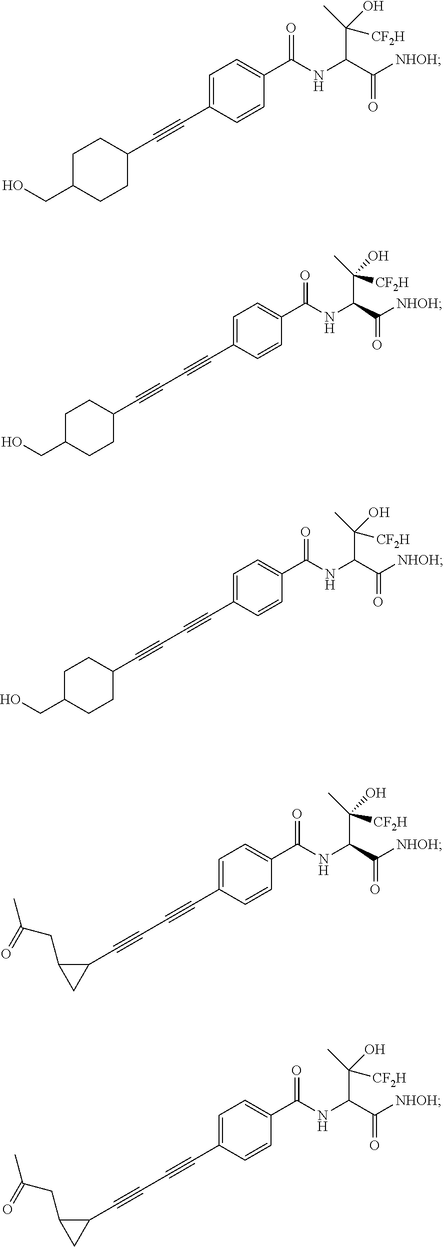 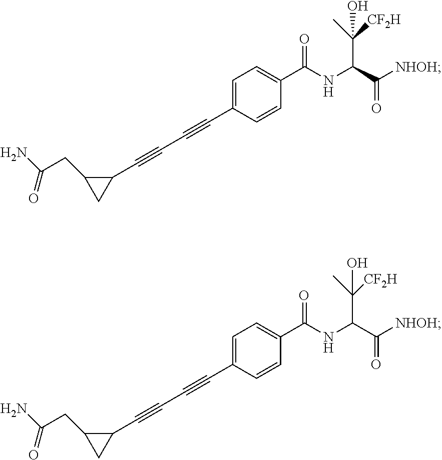 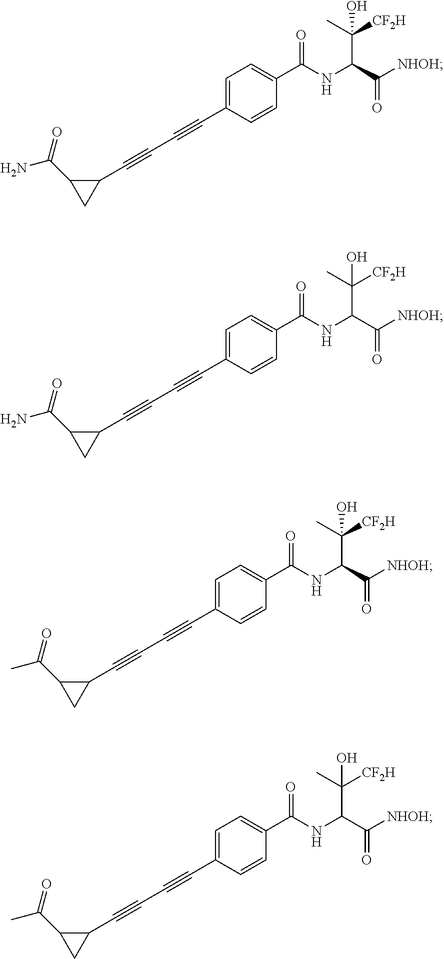 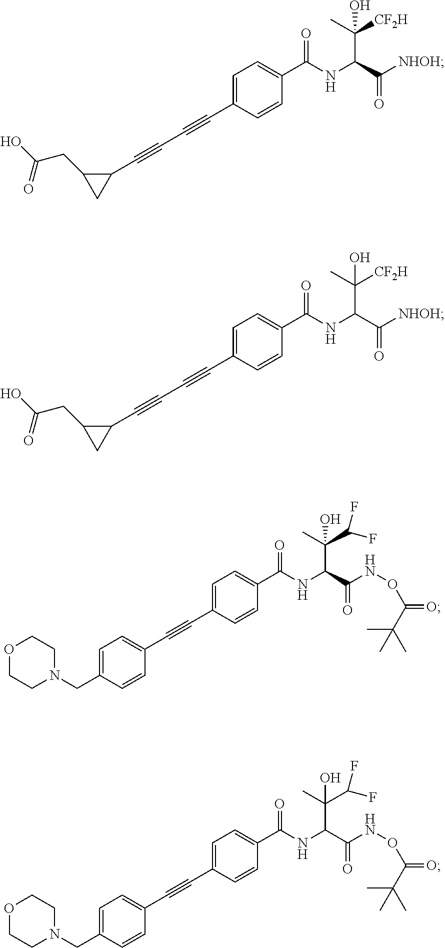 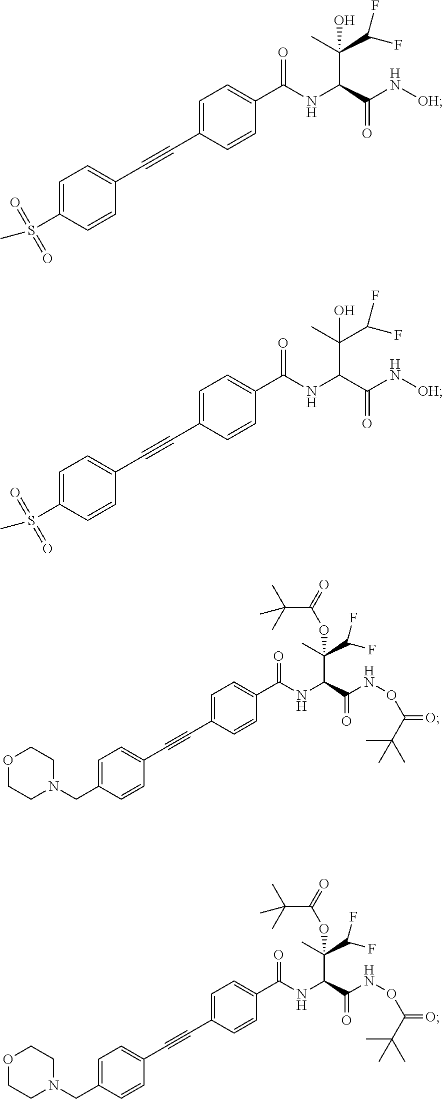 [SMILES: C[C@@](O)(C(F)F)[C@H](NC(=O)c1ccc(C#Cc2ccc(S(C)(=O)=O)cc2)cc1)C(=O)NO ; CC(O)(C(F)F)C(NC(=O)c1ccc(C#Cc2ccc(S(C)(=O)=O)cc2)cc1)C(=O)NO ; CC(C)(C)C(=O)ONC(=O)[C@@H](NC(=O)c1ccc(C#Cc2ccc(CN3CCOCC3)cc2)cc1)[C@](C)(OC(=O)C(C)(C)C)C(F)F ; CC(C)(C)C(=O)ONC(=O)[C@@H](NC(=O)c1ccc(C#Cc2ccc(CN3CCOCC3)cc2)cc1)[C@](C)(OC(=O)C(C)(C)C)C(F)F] 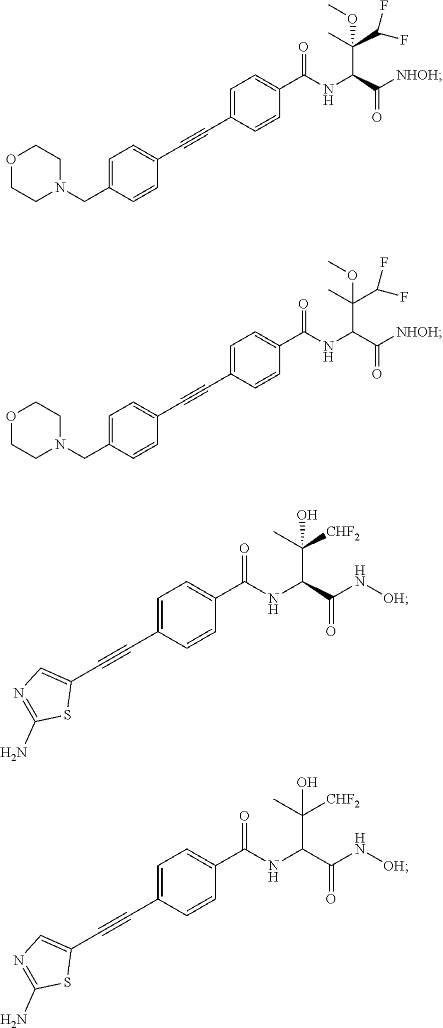 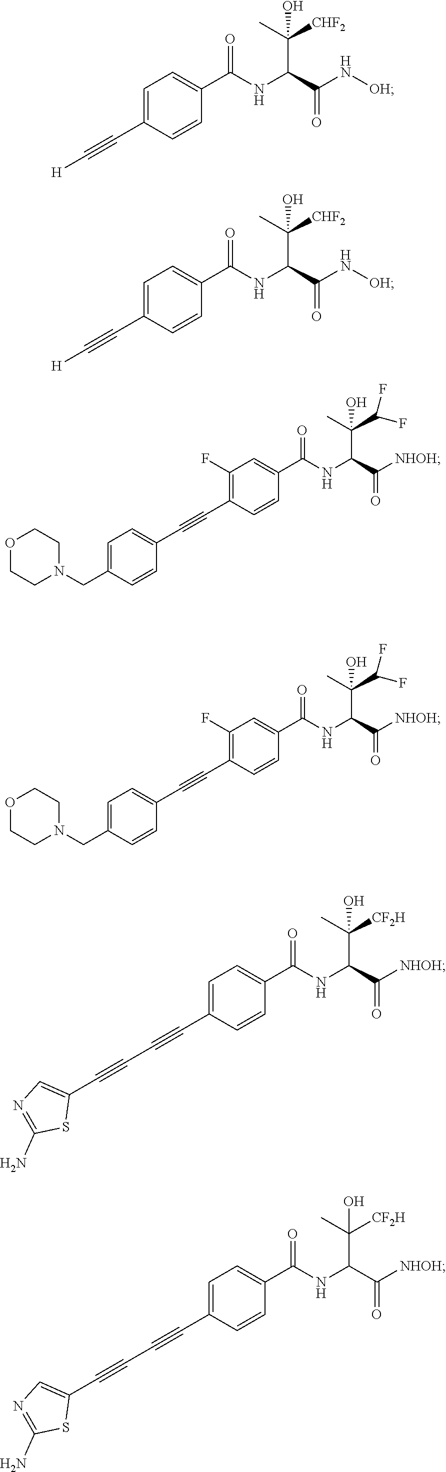 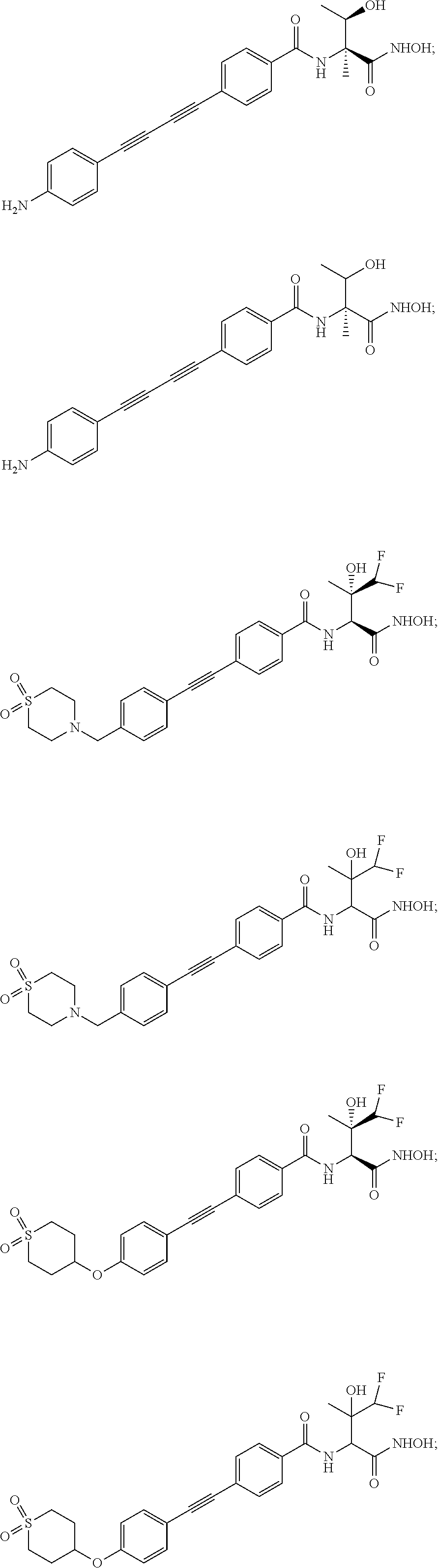 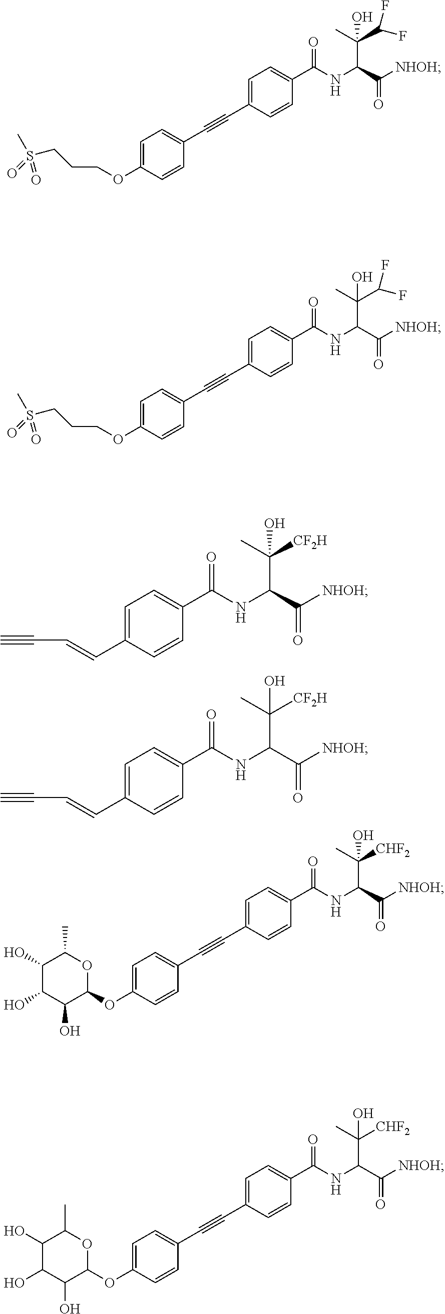
or a stereo-isomer, an enantiomer, a polymorph, a pharmaceutically acceptable salt, an ester, or a derivative thereof.

18. A pharmaceutical composition comprising a compound according to any one of claims 1-17 or a pharmaceutically acceptable salt thereof and at least one pharmaceutically acceptable vehicle, solvent, adjuvant or diluent.

19. A method of treating or preventing a Gram-negative bacterial infection in a subject, the method comprising administering to a subject in need of such treatment an effective amount of one or more compounds of any one of claims 1-17, or the composition of claim 18 such that the Gram-negative bacterial infection is treated in the subject.

20. The method according to claim 19, in which the Gram-negative bacteria is selected from the group consisting of Pseudomonas aeruginosa, Stenotrophomonas maltophilia, Burkholderia cepacia, Alcaligenes xylosoxidans, Acinetobacter, Enterobacteriaceae, Haemophilus, Neisseria, Francisella tularensis, Yersinia pestis, Burkholderia pseudomallei, Burkholderia mallei, Rickettsia prowazekii, Coxiella burnetti, Campylobacter jejuni, Shigella, Moraxella catarrhalis, Chlamydia trachomatis, and combinations thereof.

21. The method according to claim 20, in which the Gram-negative bacteria comprises Neisseria gonorrhoeae.

22. The method according to claim 20, in which the Gram-negative bacteria comprises Acinetobacter Baumannii.

23. The method according to claim 20, in which the Gram-negative bacteria comprises an Enterobacteriacacae.

24. The method according to claim 23, in which the Enterobacteriaceae is selected from the group consisting of Serratia, Proteus, Klebsiella, Enterobacter, Citrobacter, Salmonella, Providencia, Morganella, Cedecea, Edwardsiella, Escherichia coli, Enterobacter cloacae, Enterobacter aerogenes, and combinations thereof.

25. A method for inhibiting a deacetylase enzyme in Gram-negative bacteria, the method comprising contacting the bacteria with an effective amount of a compound of any one of claims 1-17 or an effective amount of the composition of claim 18.

26. The method according to claim 25, in which the deacetylase enzyme comprises LpxC.

27. A method of treating a cancer in a subject, the method comprising administering to a subject in need of such treatment an effective amount of one or more compounds as in any one of claims 1-17 or the composition of claim 18 such that the cancer is treated in the subject.

28. The method according to claim 27, in which the cancer is selected from the group consisting of breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma, peripheral neuroepithelioma, and combinations thereof.

29. The method according to any one of claims 19 to 28, the method further comprising administering to the subject one or more additional therapeutic agents.

30. The method according to claim 29, in which the one or more additional therapeutic agent is administered prior to administering the compound of any one of claims 1-17 or the pharmaceutical composition according to claim 18.

31. The method according to claim 29, in which the one or more additional therapeutic agent is administered concurrently with the compound of any one of claims 1-17 or the pharmaceutical composition according claim 18.

32. The method according to claim 29, in which the one or more additional therapeutic agent is administered after the compound of any one of claims 1-17, the pharmaceutical composition according to claim 18, or any combination thereof.

33. A kit comprising:
(1) a container holding the compound of any one of claims 1-17, the composition of claim 18, or any combination thereof; and
(2) an instruction for use;
wherein the instruction for use comprises steps in administering the compound of claims 1-17, or the composition of claim 18 to a subject in need thereof;
wherein the subject is a human subject having or is suspect of having a bacterial infection or a cancer.

34. Use of the compound of any one of claims 1-17, the composition of claim 18, or the kit of claim 33 in the manufacture of a medicament to treat a bacterial infection or a cancer.

35. The use of claim 34, wherein the bacterial infection is caused by Pseudomonas aeruginosa, Stenotrophomonas maltophilia, Burkholderia cepacia, Alcaligenes xylosoxidans, Acinetobacter, Enterobacteriaceae, Haemophilus, Neisseria, Francisella tularensis, Yersinia pestis, Burkholderia pseudomallei, Burkholderia mallei, Rickettsia prowazekii, Coxiella burnetti, Campylobacter jejuni, Shigella, Moraxella catarrhalis, Chlamydia trachomatis, and combinations thereof; or
wherein the cancer comprises breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma, peripheral neuroepithelioma, and combinations thereof.

36. Use of the compound of any one of claims 1-17, the composition of claim 18, or the kit of claim 33 in the manufacture of a medicament for inhibiting a deacetylase LpxC in a subject in need thereof.

37. The use according to claim 36, wherein the LpxC overexpression manifests a bacterial infection or a cancer.

38. The use of claim 37, wherein the bacterial infection is caused by Pseudomonas aeruginosa, Stenotrophomonas maltophilia, Burkholderia cepacia, Alcaligenes xylosoxidans, Acinetobacter, Enterobacteriaceae, Haemophilus, Neisseria, Francisella tularensis, Yersinia pestis, Burkholderia pseudomallei, Burkholderia mallei, Rickettsia prowazekii, Coxiella burnetti, Campylobacter jejuni, Shigella, Moraxella catarrhalis, Chlamydia trachomatis, and combinations thereof; or
wherein the cancer comprises breast cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulval cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma, peripheral neuroepithelioma, and combinations thereof.

39. A method of synthesizing a compound of Formula (I), the method comprising:
(a) contacting a compound of Formula (I′) 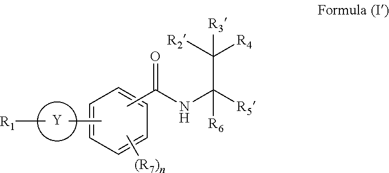
with a phosphate salt of the formula 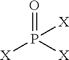
in the presence of a base catalyst to form a reaction mixture; and
(b) contacting the reaction mixture with an alkali metal hydroxide to form a compound of Formula (I): 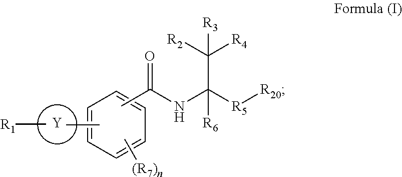
wherein
X is a halogen;
Y represents aryl optionally substituted with R₈, heteroaryl optionally substituted with R₈, cycloalkyl or heterocyclyl optionally substituted with R₈;
n is an integer 0, 1, 2, 3, or 4;
R₁ is hydrogen, C₁-C₆ alkyl optionally substituted with R₉, aryl optionally substituted with R₉, heteroaryl optionally substituted with R₉, or heterocyclyl optionally substituted with R₉;
R₂ is selected from the group consisting of halogen, C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), and amino(C₁-C₆ alkyl);
R₂′ is selected from the group consisting of halogen, C₁-C₆ alkyl, C₁-C₆haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), and amino(C₁-C₆ alkyl);
R₃ is selected from the group consisting of halogen, C₁-C₆ alkyl, C₁-C₆ haloalkyl, —NH₂, —NH(C₁-C₆ alkyl), —N(C₁-C₆ alkyl)₂, —OH, —O—R₂₀, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), and —NHCO(C₁-C₆ alkoxy);
R₃′ is selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), and —NHCO(C₁-C₆ alkoxy);
R₄ is C₁-C₆ haloalkyl;
R₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₅′ is —C₁-C₆ alkyl, —C₁-C₆ haloalkyl, —NH₂, —NH(C₁-C₆ alkyl), —NH(C₁-C₆ alkyl), —N(C₁-C₆ alkyl)₂, —OH, —C₁-C₆ alkoxy, —C₁-C₆ haloalkoxy, —CONH₂, —CONH(C₁-C₆ alkyl), —CON(C₁-C₆ alkyl), —CON(C₁-C₆ alkyl)₂, —CONH—OH, —CON(C₁-C₆ alkyl)-OH, —CONH₂, —OCO(C₁-C₆ alkyl), —O—CH(C₁-C₆ alkyl)₂, —CONH—NH₂, —CO₂H, or —CO₂(C₁-C₆ alkyl);
R₆ is hydrogen or C₁-C₆ alkyl;
each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
each R₂₀ is independently of Formula (IIa): 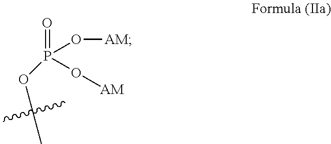
wherein each AM is independently selected from Na, Li, K, Rb, or C₁-C₆ alkyl.

40. The method of claim 39, wherein X is chloride.

41. The method of claims 39-40, wherein the base catalyst comprises a cyclic tertiary amine.

42. The method of claim 41, wherein the cyclic tertiary amine comprises a morpholine, a piperazine, a piperidine, a hydroxypiperidine, a halopiperidine, a pyrrolidine, or a N-alkyltetrahydroquinolone.

43. The method of claims 39-42, wherein the base catalyst comprises N-methylmorpholine (NMM).

44. The method of any one of claims 39-43, wherein step (a) is conducted at a temperature from about 0° C. to about −20° C.

45. The method of any one of claims 39-44, wherein the alkali metal hydroxide comprises sodium hydroxide.

46. The method of any one of claims 39-45, wherein
R₂ is selected from C₁-C₆ alkyl or —O—R₂₀;
R₂′ is selected from C₁-C₆ alkyl or —OH;
R₃ is selected from C₁-C₆ alkyl or —O—R₂₀;
R₃′ is selected from C₁-C₆ alkyl or —OH;
R₅ is selected from the group consisting of —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, and —CONH—NH—; and
R₅′ is selected from the group consisting of —CONH₂, —CONH(C₁-C₆ alkyl), —CONH(C₁-C₆ alkyl), —CON(C₁-C₆ alkyl)₂, —CONH—OH, —CON(C₁-C₆ alkyl)-OH, —CONH₂, —OCO(C₁-C₆ alkyl), and —CONH—NH₂.

47. The method of any one of claims 39-46, wherein the moiety of Formula (I) 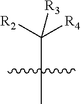
has a structure of Formula (IIIa) or Formula (IIIb):

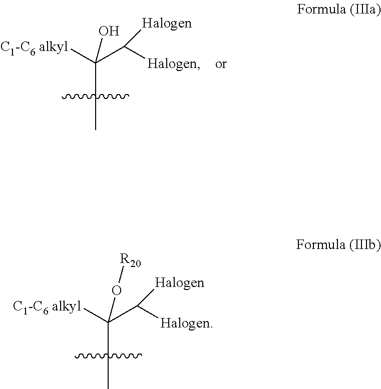

48. The method of any one of claims 39-47, wherein Y is selected from the group of phenyl, pyridinyl, pyrimidinyl, ethyleniminyl, cyclopropyl, pyrrolyl, furanyl, tetrahydrofuranyl, aziridinyl, pyrrolidinyl, pyrrolidonyl, thiophenyl, piperidinyl, thiazol, azulenyl, naphthyl, and imidazole, which are optionally substituted with R₈.

49. A method of synthesizing a compound of Formula (IV), the method comprising:
(a) contacting a compound of Formula (IV′) 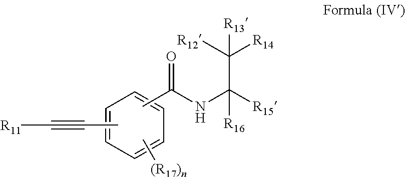
with a phosphate salt of the formula 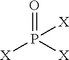
in the presence of a base catalyst to form a reaction mixture; and
(b) contacting the reaction mixture with an alkali metal hydroxide to form a compound of Formula (IV): 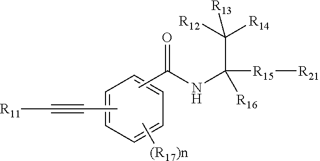
wherein
X is a halogen;
n is an integer 0, 1, 2, 3, or 4;
R₁₁ is —C≡C—R₃₃, C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈;
wherein R₃₃ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₉, (C₃-C₈ cycloalkyl) C₁-C₆ alkyl- optionally substituted with R₁₉, a C₃-C₇ cyclyl or heterocyclyl optionally substituted with R₁₉;
R₁₂ is selected from the group consisting of halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), and amino(C₁-C₆ alkyl);
R₁₂′ is selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), and amino(C₁-C₆ alkyl);
R₁₃ is selected from the group consisting of halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), and —NHCO(C₁-C₆ alkoxy);
R₁₃′ is selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), and —NHCO(C₁-C₆ alkoxy);
R₁₄ is C₁-C₆ haloalkyl;
R₁₅ is independently selected from the group consisting of —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, and —CO₂(C₁-C₆ alkyl)-;
R₁₅′ is independently selected from the group consisting of —C₁-C₆ alkyl, —C₁-C₆ haloalkyl, —NH₂, —NH(C₁-C₆ alkyl), —NH(C₁-C₆ alkyl), —N(C₁-C₆ alkyl)₂, —OH, —C₁-C₆ alkoxy, —C₁-C₆ haloalkoxy, —CONH₂, —CONH(C₁-C₆ alkyl), —CON(C₁-C₆ alkyl), —CON(C₁-C₆ alkyl)₂, —CONH—OH, —CON(C₁-C₆ alkyl)-OH, —CONH₂, —OCO(C₁-C₆ alkyl), —O—CH(C₁-C₆ alkyl)₂, —CONH—NH₂, —CO₂H, and —CO₂(C₁-C₆ alkyl);
R₁₆ is hydrogen or C₁-C₆ alkyl;
each R₁₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₁₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₁₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₁₉ groups when attached to the same carbon atom form ═O; and
each R₂₁ is independently of Formula (IIa): 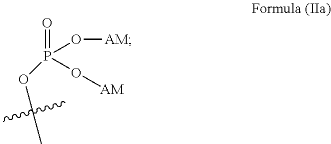
wherein each AM is independently selected from Na, Li, K, Rb, or a C₁-C₆ alkyl.

50. The method of claim 49, wherein X is chloride.

51. The method of claims 49-50, wherein the base catalyst comprises a cyclic tertiary amine.

52. The method of claim 51, wherein the cyclic tertiary amine comprises a morpholine, a piperazine, a piperidine, a hydroxypiperidine, a halopiperidine, a pyrrolidine, or a N-alkyltetrahydroquinolone.

53. The method of claims 49-52, wherein the base catalyst comprises N-Methylmorpholine (NMM).

54. The method of any one of claims 49-53, wherein step (a) is conducted at a temperature from about 0° C. to about −20° C.

55. The method of any one of claims 49-54, wherein the alkali metal hydroxide comprises sodium hydroxide.

56. The method of any one of claims 49-55, wherein
R₁₂ is selected from C₁-C₆ alkyl or —O—R₂₁;
R₁₂′ is selected from C₁-C₆ alkyl or —OH;
R₁₃ is selected from C₁-C₆ alkyl or —O—R₂₁;
R₁₃′ is selected from C₁-C₆ alkyl or —OH;
R₁₅ is selected from the group consisting of —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, and —CONH—NH—; and
R₁₅′ is selected from the group consisting of —CONH₂, —CONH(C₁-C₆ alkyl), —CONH(C₁-C₆ alkyl), —CON(C₁-C₆ alkyl)₂, —CONH—OH, —CON(C₁-C₆ alkyl)-OH, —CONH₂, —OCO(C₁-C₆ alkyl), and —CONH—NH₂.

57. The method of any one of claims 49-56 wherein the moiety of Formula (IV) 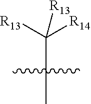
is of Formula (Va) or Formula (Vb):

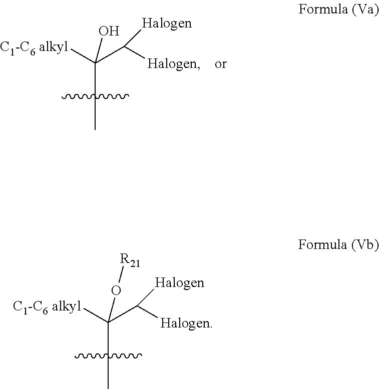

58. The method of any one of claims 49-57, wherein
R₁₁ is selected from —C≡C—R₃₃, or C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈; and
R₃₃ is —C₁-C₆ alkyl-CO₂H, C₃-C₈ cycloalkyl optionally substituted with R₁₉, (C₃-C₈ cycloalkyl) C₁-C₆ alkyl- optionally substituted with R₁₉, a C₃-C₇ cyclyl or heterocyclyl optionally substituted with R₁₉.

59. A method of synthesizing a compound of Formula (VI), the method comprising:
(a) contacting a compound of Formula (VI′) 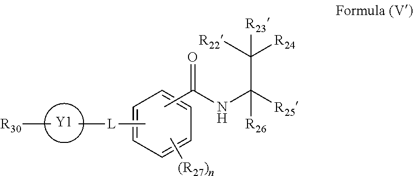
with a phosphate salt of the formula 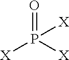
in the presence of abase catalyst to form a reaction mixture; and
(b) contacting the reaction mixture with an alkali metal hydroxide to form a compound of Formula (VI): 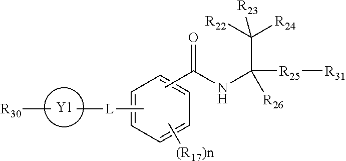
wherein
X is a halogen;
L represents a linker, wherein L is absent or is selected from the group consisting of —C≡C—C≡C—, or —C≡C—R₃₂, wherein R₃₂ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₂₉, (C₃-C₈ cycloalkyl) C₁-C₆ alkyl- optionally substituted with R₂₉, a C₃-C₇ cyclyl and heterocyclyl optionally substituted with R₂₉;
Y1 represents aryl optionally substituted with R₂₈, heteroaryl optionally substituted with R₂₈, cycloalkyl or heterocyclyl optionally substituted with R₂₈;
n is an integer 0, 1, 2, 3, or 4;
R₃₀ is hydrogen, C₁-C₆ alkyl optionally substituted with R₂₉, aryl optionally substituted with R₂₉, heteroaryl optionally substituted with R₂₉, or heterocyclyl optionally substituted with R₂₉;
R₂₂ is selected from the group consisting of halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), and amino(C₁-C₆ alkyl);
R₂₂′ is selected from the group consisting of halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), and amino(C₁-C₆ alkyl);
R₂₃ is selected from the group consisting of halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), and —NHCO(C₁-C₆ alkoxy);
R₂₃′ is selected from the group consisting of halogen, C₁-C₆ alkyl, C₁-C₆ haloalkyl, —NH₂, —NH(C₁-C₆ alkyl), —N(C₁-C₆ alkyl)₂, —OH, —C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), and —NHCO(C₁-C₆ alkoxy);
R₂₄ is C₁-C₆ haloalkyl;
R₂₅ is independently selected from the group consisting of —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, and —CO₂(C₁-C₆ alkyl)-;
R₂₅′ is independently selected from the group consisting of —C₁-C₆ alkyl, —C₁-C₆ haloalkyl, —NH₂, —NH(C₁-C₆ alkyl), —NH(C₁-C₆ alkyl), —N(C₁-C₆ alkyl)₂, —OH, —C₁-C₆ alkoxy, —C₁-C₆ haloalkoxy, —CONH₂, —CONH(C₁-C₆ alkyl), —CON(C₁-C₆ alkyl)₂, —CONH—OH, —CONH(C₁-C₆ alkyl)-OH, —CONH₂, —OCO(C₁-C₆ alkyl), —O—CH(C₁-C₆ alkyl)₂, —CONH—NH₂, —CO₂H, and —CO₂(C₁-C₆ alkyl);
R₂₆ is hydrogen or C₁-C₆ alkyl;
each R₂₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₂₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
each R₃₁ is independently a compound of Formula (IIa): 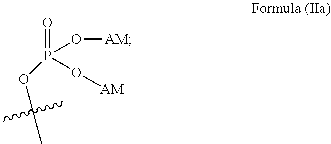
wherein each AM is independently selected from Na, Li, K, Rb, or a C₁-C₆ alkyl.

60. The method of claim 59, wherein X is chloride.

61. The method of claims 59-60, wherein the base catalyst comprises a cyclic tertiary amine.

62. The method of claim 61, wherein the cyclic tertiary amine comprises a morpholine, a piperazine, a piperidine, a hydroxypiperidine, a halopiperidine, a pyrrolidine, or a N-alkyltetrahydroquinolone.

63. The method of claims 59-62, wherein the base catalyst comprises N-Methylmorpholine (NMM).

64. The method of any one of claims 59-63, wherein step (a) is conducted at a temperature from about 0° C. to about −20° C.

65. The method of any one of claims 59-64, wherein the alkali metal hydroxide comprises sodium hydroxide.

66. The method of any one of claims 59-65, wherein
R₂₂ is selected from C₁-C₆ alkyl or —O—R₃₁;
R₂₂′ is selected from C₁-C₆ alkyl or —OH;
R₂₃ is selected from C₁-C₆ alkyl or —O—R₃₁;
R₂₃′ is selected from C₁-C₆ alkyl or —OH;
R₂₅ is selected from the group consisting of —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, and —CONH—NH—; and
R₂₅′ is selected from the group consisting of —CONH₂, —CONH(C₁-C₆ alkyl), —CONH(C₁-C₆ alkyl), —CON(C₁-C₆ alkyl)₂, —CONH—OH, —CON(C₁-C₆ alkyl)-OH, —CONH₂, —OCO(C₁-C₆ alkyl), and —CONH—NH₂.

67. The method of claims 59-66, wherein the moiety of Formula (VI) 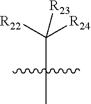
is of Formula (VIIa) or Formula (VIIb):

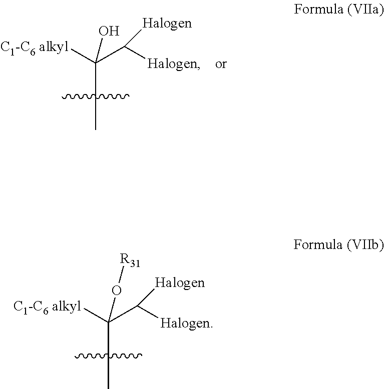

68. The method of any one of claims 59-67, wherein Y₁ is selected from the group of phenyl, pyridinyl, pyrimidinyl, ethyleniminyl, cyclopropyl, pyrrolyl, furanyl, tetrahydrofuranyl, aziridinyl, pyrrolidinyl, pyrrolidonyl, thiophenyl, piperidinyl, thiazol, azulenyl, naphthyl, and imidazole, which are optionally substituted with R₂₈.

69. A method of synthesizing a compound of Formula (I), comprising contacting a compound of Formula (I″) 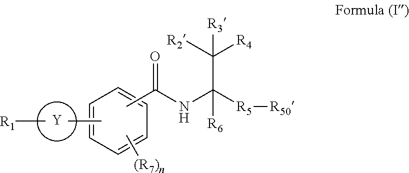
with a) a monophosphate kinase, b) a mixture of H₃PO₄ and P₄O₁₀, c) P₄O₁₀ alone, or d) a mixture of p-toluenesulfonyl chloride (TsCl) and pyridine to form a compound of Formula (I) 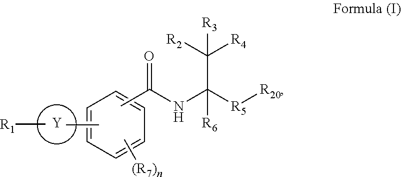
wherein Y represents aryl optionally substituted with R₈, heteroaryl optionally substituted with R₈, cycloalkyl, or heterocyclyl optionally substituted with R₈;
X is a halogen;
n is an integer selected from 0, 1, 2, 3, or 4;
R₁ is hydrogen, C₁-C₆ alkyl optionally substituted with R₉, aryl optionally substituted with R₉, heteroaryl optionally substituted with R₉, or heterocyclyl optionally substituted with R₉;
R₂ is halogen, C₁-C₆ alkyl, C₁-C₆haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₃ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₃′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₄ is C₁-C₆ haloalkyl;
R₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₅₀′ is H or Formula (IIa) 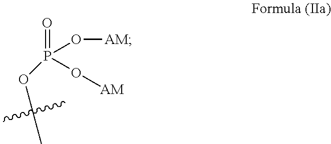
R₆ is hydrogen or C₁-C₆ alkyl;
each R₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups that when attached to the same carbon atom form ═O;
each R₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups that when attached to the same carbon atom form ═O; and
R₂₀ is of Formula (IIb): 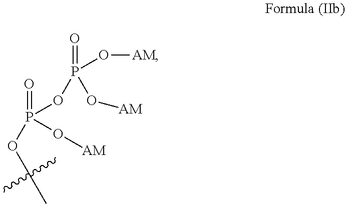
wherein each AM is independently selected from Na, Li, K, Rb, or a C₁-C₆ alkyl.

70. A method of synthesizing a compound of Formula (IV) comprising contacting a compound of Formula (IV″) 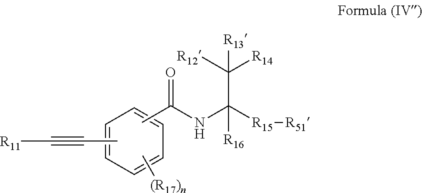
with a) a monophosphate kinase, b) a mixture of H₃PO₄ and P₄O₁₀, c) P₄O₁₀ alone, or d) a mixture of p-toluenesulfonyl chloride (TsCl) and pyridine to form a compound of Formula (IV) 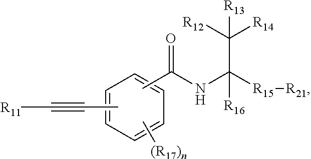
wherein n is an integer selected from 0, 1, 2, 3, or 4;
R₁₁ is —C≡C—R₃₃, C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₈, (C₃-C₈ cycloalkyl) C₁-C₆ alkyl- optionally substituted with R₁₈, or heterocyclyl optionally substituted with R₁₈;
R₃₃ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₁₉, (C₃-C₈ cycloalkyl) C₁-C₆ alkyl-optionally substituted with R₁₉, a C₃-C₇ cyclyl or heterocyclyl optionally substituted with R₁₉;
R₁₂ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₂′ is halogen, C₁-C₆ alkyl, C₁-C₆haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₁₃ is halogen, C₁-C₆ alkyl, C₁-C₆ haloalkyl, —NH₂, —NH(C₁-C₆ alkyl), —N(C₁-C₆ alkyl)₂, —OH, —O—R₂₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₃′ is halogen, C₁-C₆ alkyl, C₁-C₆ haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₁₄ is C₁-C₆ haloalkyl;
R₅₁′ is H or Formula (IIa) 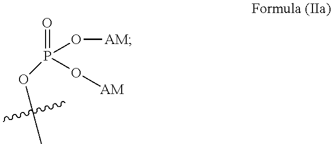
R₁₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—₁—OCO(C₁-C₆ alkyl)-, —O—C(C₁-C₆ alkyl)₂-, —CONH—NH—, —CO₂—, or —CO₂(C₁-C₆ alkyl)-;
R₁₆ is hydrogen or C₁-C₆ alkyl;
each R₁₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₁₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups that when attached to the same carbon atom form ═O;
each R₁₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₁₉ groups that when attached to the same carbon atom form ═O; and
R₂₁ is Formula (IIb): 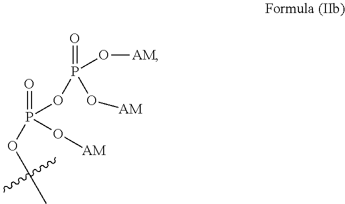
wherein each AM is independently selected from Na, Li, K, Rb, or a C₁-C₆ alkyl. In an exemplary embodiment, each AM is independently Na or methyl.

71. A method of synthesizing a compound of Formula (VI) comprising contacting a compound of Formula (VI″) 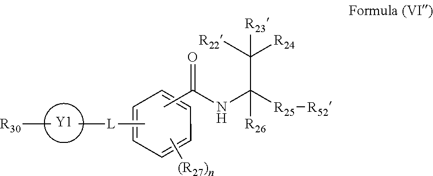
with a) a monophosphate kinase, b) a mixture of H₃PO₄ and P₄O₁₀, c) P₄O₁₀ alone, or d) a mixture of p-toluenesulfonyl chloride (TsCl) and pyridine to form a compound of Formula (VI) 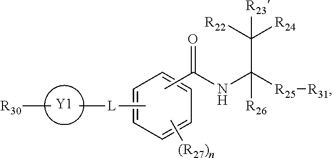
wherein
L represents a linker, wherein L is absent or is selected from the group consisting of —C≡C—C≡C—, or —C≡C—R₃₂, wherein R₃₂ is C₁-C₆ alkyl, C₁-C₆ haloalkyl, —CO(C₁-C₆ alkyl), —C₁-C₆ alkyl-CO(C₁-C₆ alkyl), —C₁-C₆ alkyl-CO₂H, C₃-C₈ cycloalkyl optionally substituted with R₂₉, (C₃-C₈ cycloalkyl) C₁-C₆ alkyl- optionally substituted with R₂₉, a C₃-C₇ cyclyl and heterocyclyl optionally substituted with R₂₉;
Y1 represents aryl optionally substituted with R₂₈, heteroaryl optionally substituted with R₂₉, cycloalkyl, or heterocyclyl optionally substituted with R₂₈;
n is an integer selected from 0, 1, 2, 3, or 4;
R₃₀ is hydrogen, C₁-C₆ alkyl optionally substituted with R₂₉, aryl optionally substituted with R₂₉, heteroaryl optionally substituted with R₂₉, or heterocyclyl optionally substituted with R₂₉;
R₂₂ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₂′ is halogen, C₁-C₆ alkyl, C₁-C₆haloalkyl, —NH₂, —NH(C₁-C₆ alkyl), —N(C₁-C₆ alkyl)₂, —OH, C₁-C₆ alkoxy, C₁-C₆ haloalkoxy, —SH, —S(C₁-C₆ alkyl), hydroxy(C₁-C₆ alkyl), alkoxy(C₁-C₆ alkyl), or amino(C₁-C₆ alkyl);
R₂₃ is halogen, C₁-C₆ alkyl, C₁-C₆ haloalkyl, —NH₂, —NH(C₁-C₆ alkyl), —N(C₁-C₆ alkyl)₂, —OH, —O—R₃₁, C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₃′ is halogen, C₁-C₆ alkyl, C₁-C₆ haloalkyl, —NH₂, —NH(C₁-C₆ alkyl), —N(C₁-C₆ alkyl)₂, —OH, —C₁-C₆ alkoxy, C₁-C₆ haloalkoxy, —SH, —S(C₁-C₆ alkyl), hydroxy(C₁-C₆ alkyl), alkoxy(C₁-C₆ alkyl), amino(C₁-C₆ alkyl), —NHCO(C₁-C₆ alkyl), —NHCONH₂, —NHCONH(C₁-C₆ alkyl), —OCO(C₁-C₆ alkyl), or —NHCO(C₁-C₆ alkoxy);
R₂₄ is C₁-C₆ haloalkyl;
R₂₅ is —C₁-C₆ alkyl-, —C₁-C₆ haloalkyl-, —NH—, —NH(C₁-C₆ alkyl)-, —N(C₁-C₆ alkyl)-, —N(C₁-C₆ alkyl)₂-, —O—, —C₁-C₆ alkoxy-, —C₁-C₆ haloalkoxy-, —CONH—, —CONH(C₁-C₆ alkyl)-, —CON(C₁-C₆ alkyl)-, —CON(C₁-C₆ alkyl)₂-, —CONH—O—, —CON(C₁-C₆ alkyl)-O—, —CONH—, —OCO(C₁-C₆ alkyl)-, —O—C(C₁-C₆ alkyl)₂-, —CONH—NH—, —CO₂—, or —CO₂(C₁-C₆ alkyl)-;
R₅₂′ is H or Formula (IIa) 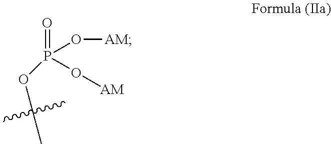
R₂₆ is hydrogen or C₁-C₆ alkyl;
each R₂₇ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy;
each R₂₈ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, d-C₆ haloalkoxy, —CONH₂, —CONH(C₁-C₆ alkyl), —CON(C₁-C₆ alkyl)₂, —CONH—OH, —CONH—OCO(C₁-C₆ alkyl), —C(NH)NH—OH, —CONH—NH₂, —CO₂H, and —CO₂(C₁-C₆ alkyl); or two R₈ groups when attached to the same carbon atom form ═O;
each R₂₉ is independently selected from the group consisting of halogen, —NO₂, —CN, C₁-C₆ alkyl, C₁-C₆ haloalkyl, —NH₂, —NH(C₁-C₆ alkyl), —N(C₁-C₆ alkyl)₂, —OH, C₁-C₆ alkoxy, and C₁-C₆ haloalkoxy; or two R₉ groups when attached to the same carbon atom form ═O; and
each R₃₁ is Formula (IIb);
R₂₀ is of Formula (IIb); 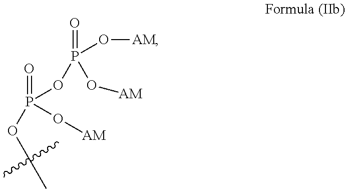
wherein each AM is independently selected from Na, Li, K, Rb, or a C₁-C₆ alkyl.
